SEQUENCE LISTING (40 sequences)

SEQ ID NO: 1 - protein, 16 aa, Homo sapiens
  MISC_FEATURE (2)..(2): Can be any amino acid residue
PXCLLVXRCGGXCXCC

SEQ ID NO: 2 - DNA, 2108 bp, Homo sapiens
  misc_feature (2002)..(2002): can be any of a, c, g, or t
ccccgccgtgagtgagctctcaccccagtcagccaaatgagcctcttcgggcttctcctggtgacatctgccctggccggccagagacgagggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcrtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatggatataaaaatattgttgccctggacaaaaatacatgtatntccatcccggaatggtgctagagttggattaaacctgcattttaaaaaacctgaattgggaanggaanttggtaaggttggccaaancttttttgaaaataattaa

SEQ ID NO: 3 - protein, 345 aa, Homo sapiens [sequence omitted]

SEQ ID NO: 4 - DNA, 1536 bp, Homo sapiens
cgggtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaasaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcatgctgataggacagactgga

SEQ ID NO: 5 - protein, 318 aa, Homo sapiens
GKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 6 - DNA, 1474 bp, Murinae gen. sp.
  misc_feature (1447)..(1447): can be any of a, c, g, or t
cacctggagacacagaagagggctctaggaaaaattttggatggggattatgtggaaactaccctgcgattctctgctgccagagccggccaggcgcttccaccgcagcgcagcctttccccgggctgggctgagccttggagtcgtcgcttccccagtgcccgccgcgagtgagccctcgccccagtcagccaaatgctcctcctcggcctcctcctgctgacatctgccctggccggccaaagaacggggactcgggctgagtccaacctgagcagcaagttgcagctctccagcgacaaggaacagaacggagtgcaagatccccggcatgagagagttgtcactatatctggtaatgggagcatccacagcccgaagtttcctcatacgtacccaagaaatatggtgctggtgtggagattagttgcagtagatgaaaatgtgcggatccagctgacatttgatgagagatttgggctggaagatccagaagacgatatatgcaagtatgattttgtagaagttgaggagcccagtgatggaagtgttttaggacgctggtgtggttctgggactgtgccaggaaagcagacttctaaaggaaatcatatcaggataagatttgtatctgatgagtattttccatctgaacccggattctgcatccactacagtattatcatgccacaagtcacagaaaccacgagtccttcggtgttgcccccttcatctttgtcattggacctgctcaacaatgctgtgactgccttcagtaccttggaagagctgattcggtacctagagccagatcgatggcaggtggacttggacagcctctacaagccaacatggcagcttttgggcaaggctttcctgtatgggaaaaaaagcaaagtggtgaatctgaatctcctcaaggaagaggtaaaactctacagctgcacaccccggaacttctcagtgtccatacgggaagagctaaagaggacagataccatattctggccaggttgtctcctggtcaagcgctgtggaggaaattgtgcctgttgtctccataattgcaatgaatgtcagtgtgtcccacgtaaagttacaaaaaagtaccatgaggtccttcagttgagaccaaaaactggagtcaagggattgcataagtcactcactgatgtggctctggaacaccacgaggaatgtgactgtgtgtgtagaggaaacgcaggagggtaactgcagccttcgtagcagcacacgtgagcactggcattctgtgtacccccacaagcaaccttcatccccaccagcgttggccgcagggctctcagctgctgatgctggctatggtaaagatcttactcgtctccaaccaaattctcagttgtttgcttcaatagccttcccctgcaggacttcaagtgtcttctaaaagaccagaggcaccaanaggagtcaatcacaaagcactgcaccg

SEQ ID NO: 7 - protein, 345 aa, Murinae gen. sp.
MLLLGLLLLTSALAGQRTGTRAESNLSSKLQLSSDKEQNGVQDPRHERVVTISGNGSIHSPKFPHTYPRNMVLVWRLVAVDENVRIQLTFDERFGLEDPEDDICKYDFVEVEEPSDGSVLGRWCGSGTVPGKQTSKGNHIRIRFVSDEYFPSEPGFCIHYSIIMPQVTETTSPSVLPPSSLSLDLLNNAVTAFSTLEELIRYLEPDRWQVDLDSLYKPTWQLLGKAFLYGKKSKVVNLNLLKEEVKLYSCTPRNFSVSIREELKRTDTIFWPGCLLVKRCGGNCACCLHNCNECQCVPRKVTKKYHEVLQLRPKTGVKGLHKSLTDVALEHHEECDCVCRGNAGG

SEQ ID NO: 8 - protein, 192 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSSERRKHLFVQDPQTCKCSCKNTDSRCKARQLELNERTCRCDKPRR

SEQ ID NO: 9 - protein, 170 aa, Homo sapiens [sequence omitted]

SEQ ID NO: 10 - protein, 188 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 11 - protein, 133 aa, Homo sapiens
MKLLVGILVAVCLHQYLLNADSNTKGWSEVLKGSECKPRPIVVPVSETHPELTSQRFNPPCVTLMRCGGCCNDESLECVPTEEVNVSMELLGASGSGSNGMQRLSFVEHKKCDCRPRFTTTPPTTTRPPRRRR

SEQ ID NO: 12 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 13 - protein, 358 aa, Homo sapiens
MYGEWGMGNILMMFHVYLVQGFRSEHGPVKDFSFERSSRSMLERSEQQIRAASSLEELLQIAHSEDWKLWRCRLKLKSLASMDSRSASHRSTRFAATFYDTETLKVIDEEWQRTQCSPRETCVEVASELGKTTNTFFKPPCVNVFRCGGCCNEEGVMCMNTSTSYISKQLFEISVPLTSVPELVPVKIANHTGCKCLPTGPRHPYSIIRRSIQTPEEDECPHSKKLCPIDMLWDNTKCKCVLQDETPLPGTEDHSYLQEPTLCGPHMTFDEDRCECVCKAPCPGDLIQHPENCSCFECKESLESCCQKHKIFHPDTCSCEDRCPFHTRTCASRKPACGKHWRFPKETRAQGLYSQENP

SEQ ID NO: 14 - protein, 211 aa, Homo sapiens [sequence omitted]

SEQ ID NO: 15 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 16 - protein, 182 aa, Homo sapiens
MPQFTDCVCRGSTGGEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEEC

SEQ ID NO: 17 - protein, 182 aa, Murinae gen. sp.
MPQVTETTSPSVLPPSSLSLDLLNNAVTAFSTLEELIRYLEPDRWQVDLDSLYKPTWQLDCVCRGNAGGLGKAFLYGKKSKVVNLNLLKEEVKLYSCTPRNFSVSIREELKRTDTIFWPGCLLVKRCGGNCACCLHNCNECQCVPRKVTKKYHEVLQLRPKTGVKGLHKSLTDVALEHHEEC

SEQ ID NO: 18 - protein, 117 aa, Murinae gen. sp.
ERVVTISGNGSIHSPKFPHTYPRNMVLVWRLVAVDENVRIQLTFDERFGLEDPEDDICKYDFVEVEEPSDGSVLGRWCGSGTVPGKQTSKGNMIRIRFVSDEYFPSEPGFCIHYSII

SEQ ID NO: 19 - protein, 117 aa, Homo sapiens
ERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIV

SEQ ID NO: 20 - protein, 113 aa, Homo sapiens
CGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAI

SEQ ID NO: 21 - protein, 112 aa, Homo sapiens
CGGDVKKDYGHIQSPNYPDDYRPSKVCIWRIQVSEGFHVGLTFQSFEIERMDSCAYDYLEVRDGHSESSTLIGRYCGYEKPDDIKSTSSRLWLKFVSDGSINKAGFAVNFFK

SEQ ID NO: 22 - protein, 113 aa, Homo sapiens
CGGFLTKLNGSITSPGWPKEYPPNKNCIWQLVAPTQYRISLQFDFFETEGNDVCKYDFVEVRSGLTADSKLHGKFCGSEKPEVITSQYNNMRVEPKSDNTVSKKGFKAHFFSE

SEQ ID NO: 23 - protein, 113 aa, Homo sapiens
GDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEI

SEQ ID NO: 24 - protein, 119 aa, Homo sapiens
CSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVL

SEQ ID NO: 25 - DNA, 19 bp, Homo sapiens
gaagttgaggaacccagtg

SEQ ID NO: 26 - DNA, 20 bp, Homo sapiens
cttgccaagaagttgccaag

SEQ ID NO: 27 - DNA, 19 bp, Murinae gen. sp.
cttcagtaccttggaagag

SEQ ID NO: 28 - DNA, 19 bp, Murinae gen. sp.
cgcttgaccaggagacaac

SEQ ID NO: 29 - DNA, 30 bp, Murinae gen. sp.
acgtgaattcagcaagttcagcctggttaa

SEQ ID NO: 30 - DNA, 30 bp, Murinae gen. sp.
acgtggatcctgagtatttcttccagggta

SEQ ID NO: 31 - protein, 22 aa, Homo sapiens
CKFQFSSNKEQNGVQDPQHERC

SEQ ID NO: 32 - protein, 21 aa, Homo sapiens
GRKSRVVDLNLLTEEVRLYSC

SEQ ID NO: 33 - DNA, 26 bp, Homo sapiens
cgggatcccgaatccaacctgagtag

SEQ ID NO: 34 - DNA, 61 bp, Homo sapiens
ggaattcctaatggtgatggtgatgatgtttgtcatcgtcatctcctcctgtgctccctct

SEQ ID NO: 35 - DNA, 29 bp, Homo sapiens
cggatcccggaagaaaatccagagtggtg

SEQ ID NO: 36 - DNA, 61 bp, Homo sapiens; identical to SEQ ID NO: 34

SEQ ID NO: 37 - protein, 21 aa, Homo sapiens
GRKSRVVDLNLLTEEVRLYSC

SEQ ID NO: 38 - DNA, 26 bp, Homo sapiens
  misc_feature: Forward PCR primer from the human PDGF-C 430 bp cDNA fragment encoding the CUB domain which includes a BamHI site
cgggatcccgaatccaacctgagtag

SEQ ID NO: 39 - DNA, 60 bp, Homo sapiens
  misc_feature: Reverse PCR primer from the human PDGF-C 430 bp cDNA fragment encoding the CUB domain which includes a EcoRI site and sequences coding for a C-terminal 6X His tag preceded by an enterokinase site
ccggaattcctaatggtgatggtgatgatgtttgtcatcgtcgtcgacaatgttgtagtg

SEQ ID NO: 40 - protein, 116 aa, Artificial Sequence
  PDGF-C core domain
GRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

(19) United States
(12) Patent Application Publication
(10) Pub. No.: US 2004/0053837 A1
(43) Pub. Date: Mar. 18, 2004

(54) Composition and method for modulating vasculogenesis or angiogenesis

(75) Inventors: Xuri Li, Stockholm (SE); Ulf Eriksson, Stockholm (SE); Peter Carmeliet, Leuven (BE); Desire?apos; Collen, Leuven (BE)

Correspondence Address: CROWELL & MORING LLP, INTELLECTUAL PROPERTY GROUP, P.O. BOX 14300, WASHINGTON, DC, 20044-4300, US

(73) Assignee: Ludwig Institute for Cancer Research

(21) Appl. No.: 10/439,337

(22) Filed: May 16, 2003

Related U.S. Application Data
(63) Continuation-in-part of application No. 10/303,997, filed on Nov. 26, 2002.
(63) Continuation-in-part of application No. 09/410,349, filed on Sep. 30, 1999.
(60) Provisional application No. 60/102,461, filed on Sep. 30, 1998.
(60) Provisional application No. 60/108,109, filed on Nov. 12, 1998.
(60) Provisional application No. 60/110,749, filed on Dec. 3, 1998.
(60) Provisional application No. 60/113,002, filed on Dec. 18, 1998.
(60) Provisional application No. 60/135,426, filed on May 21, 1999.
(60) Provisional application No. 60/144,022, filed on Jul. 15, 1999.

(51) Int. Cl. A61K 38/18

(52) U.S. Cl. 514/12

(57) ABSTRACT

A method for modulating vasculogenesis or arteriogenesis or angiogenesis, especially for treating heart and limb ischemia, using the core domain protein of PDGF-C, a new member of the PDGF/VEGF family of growth factors, or a homodimer or a heterodimer comprising the core domain. Also disclosed are pharmaceutical compositions comprising the core protein, nucleotide sequences encoding the protein, and uses thereof in medical and diagnostic applications.

### CROSS REFERENCE TO RELATED APPLICATIONS

This application is a Continuation-In-Part Application of pending U.S. patent application Ser. No. 10/303,979, which is a Continuation-In-Part Application of pending U.S. patent application Ser. No. 09/410,349, filed Sep. 30, 1999, which claims the benefit of U.S. Provisional Application No. 60/102,461, filed Sep. 30, 1998; U.S. Provisional Application No. 60/108,109, filed Nov. 12, 1998; U.S. Provisional Application No. 60/110,749, filed Dec. 3, 1998; U.S. Provisional Application No. 60/113,002, filed Dec. 18, 1998; U.S. Provisional Application No. 60/135,426, filed May 21, 1999; and U.S. Provisional Application No. 60/144,022, filed Jul. 15, 1999.

### FIELD OF THE INVENTION

This invention relates to growth factors for connective tissue cells, fibroblasts, myofibroblasts and glial cells and/or to growth factors for endothelial cells, and in particular to a novel platelet-derived growth factor/vascular endothelial growth factor-like growth factor, a polynucleotide sequence encoding the factor, and to pharmaceutical and diagnostic compositions and methods utilizing or derived from the factor. In particular, this invention relates to the use of the factor for enhancing post-ischemic revascularization in the heart and limb by mobilizing endothelial progenitor cells, inducing differentiation of bone marrow cells into endothelial progenitor cells, stimulating migration of endothelial cells, and/or upregulating VEGF expression.

### BACKGROUND OF THE INVENTION

In the developing embryo, the primary vascular network is established by in situ differentiation of mesodermal cells in a process called vasculogenesis. Vasculogenesis, the de novo formation of blood vessels from progenitor stem cells, can also occur in adults which involves the mobilization and differentiation of vascular progenitor, for example, from the bone marrow to sites of active vessel growth. It is believed that all subsequent processes involving the generation of new vessels in the embryo and neovascularization in adults, are governed by the sprouting or splitting of new capillaries from the pre-existing vasculature in a process called angiogenesis (Pepper et al., Enzyme & Protein, 1996 49:138-162; Breier et al., Dev. Dyn. 1995 204:228-239; Risau, Nature, 1997 386:671-674). Angiogenesis is not only involved in embryonic development and normal tissue growth, repair, and regeneration, but is also involved in the female reproductive cycle, establishment and maintenance of pregnancy, and in repair of wounds and fractures. Arteriogenesis, the formation of large bore vessel containing smooth muscle cells, is thought to be a continuum of the angiogenic process.

In addition to angiogenesis which takes place in the normal individual, angiogenic events are involved in a number of pathological processes, notably tumor growth and metastasis, and other conditions in which blood vessel proliferation, especially of the microvascular system, is increased, such as diabetic retinopathy, psoriasis and arthropathies. Inhibition of angiogenesis is useful in preventing or alleviating these pathological processes. On the other hand, promotion of angiogenesis is desirable in situations where vascularization is to be established or extended, for example after tissue or organ transplantation, or to stimulate establishment of perivascular and/or collateral circulation in tissue infarction or arterial stenosis, such as in coronary heart disease and thromboangitis obliterans. All three processes of new blood vessel formation—angiogenesis, arteriogenesis, and vasculogenesis—are play a role in the response to ischemia.

The angiogenic process is highly complex and involves the maintenance of the endothelial cells in the cell cycle, degradation of the extracellular matrix, migration and invasion of the surrounding tissue and finally, tube formation. The molecular mechanisms underlying the complex angiogenic processes are far from being understood.

Because of the crucial role of angiogenesis in so many physiological and pathological processes, factors involved in the control of angiogenesis have been intensively investigated. A number of growth factors have been shown to be involved in the regulation of angiogenesis; these include fibroblast growth factors (FGFs), platelet-derived growth factor (PDGF), transforming growth factor alpha (TGFα), and hepatocyte growth factor (HGF). See for example Folkman et al., J. Biol. Chem., 1992 267 10931-10934 for a review.

It has been suggested that a particular family of endothelial cell-specific growth factors, the vascular endothelial growth factors (VEGFs), and their corresponding receptors is primarily responsible for stimulation of endothelial cell growth and differentiation, and for certain functions of the differentiated cells. These factors are members of the PDGF/VEGF family, and appear to act primarily via endothelial receptor tyrosine kinases (RTKs).

Nine different proteins have been identified in the PDGF/VEGF family, namely two PDGFs (A and B), VEGF and six members that are closely related to VEGF. The six members closely related to VEGF are: VEGF-B, described in International Patent Application PCT/US96/02957 (WO 96/26736) and in U.S. Pat. Nos. 5,840,693 and 5,607,918 by Ludwig Institute for Cancer Research and The University of Helsinki; VEGF-C, described in Joukov et al., EMBO J., 1996 15 290-298 and Lee et al., Proc. Natl. Acad. Sci. USA, 1996 93 1988-1992; VEGF-D, described in International Patent Application No. PCT/US97/14696 (WO 98/07832), and Achen et al., Proc. Natl. Acad. Sci. USA, 1998 95 548-553; the placenta growth factor (PlGF), described in Maglione et al., Proc. Natl. Acad. Sci. USA, 1991 88 9267-9271; VEGF2, described in International Patent Application No. PCT/US94/05291 (WO 95/24473) by Human Genome Sciences, Inc; and VEGF3, described in International Patent Application No. PCT/US95/07283 (WO 96/39421) by Human Genome Sciences, Inc.

Each VEGF family member has between 30% and 45% amino acid sequence identity with VEGF. The VEGF family members share a VEGF homology domain which contains the six cysteine residues which form the cysteine knot motif. Functional characteristics of the VEGF family include varying degrees of mitogenicity for endothelial cells, induction of vascular permeability and angiogenic and lymphangiogenic properties.

Vascular endothelial growth factor (VEGF) is a homodimeric glycoprotein that has been isolated from several sources. VEGF shows highly specific mitogenic activity for endothelial cells. VEGF has important regulatory functions in the formation of new blood vessels during embryonic vasculogenesis and in angiogenesis during adult life (Carmeliet et al., Nature, 1996 380:435-439; Ferrara et al., Nature, 1996 380:439-442; reviewed in Ferrara and Davis-Smyth, Endocrine Rev., 1997 18:4-25). The significance of the role played by VEGF has been demonstrated in studies showing that inactivation of a single VEGF allele results in embryonic lethality due to failed development of the vasculature (Carmeliet et al., Nature, 1996 380:435-439; Ferrara et al., Nature, 1996 380:439-442).

In addition VEGF has strong chemoattractant activity towards monocytes, can induce the plasminogen activator and the plasminogen activator inhibitor in endothelial cells, and can also induce microvascular permeability. Because of the latter activity, it is sometimes referred to as vascular permeability factor (VPF). The isolation and properties of VEGF have been reviewed; see Ferrara et al., J. Cellular Biochem., 1991 47 211-218 and Connolly, J. Cellular Biochem., 1991 47 219-223. Alterative mRNA splicing of a single VEGF gene gives rise to five isoforms of VEGF.

VEGF-B has similar angiogenic and other properties to those of VEGF, but is distributed and expressed in tissues differently from VEGF. In particular, VEGF-B is very strongly expressed in heart, and only weakly in lung, whereas the reverse is the case for VEGF. This suggests that VEGF and VEGF-B, despite the fact that they are co-expressed in many tissues, may have functional differences.

A comparison of the PDGF/VEGF family of growth factors reveals that the 167 amino acid isoform of VEGF-B is the only family member that is completely devoid of any glycosylation. Gene targeting studies have shown that VEGF-B deficiency results in mild cardiac phenotype, and impaired coronary vasculature (Bellomo et al., Circ. Res. 2000 86:E29-35). VEGF-B knock out mice were demonstrated to have impaired coronary vessel structure, smaller hearts and impaired recovery after cardiac ischemia (Bellomo, D. et al., Circulation Research (Online), 2000 86:E29-35).

Human VEGF-B was isolated using a yeast co-hybrid interaction trap screening technique by screening for cellular proteins which might interact with cellular retinoic acid-binding protein type I (CRABP-I). The isolation and characteristics including nucleotide and amino acid sequences for both the human and mouse VEGF-B are described in detail in PCT/US96/02957, in U.S. Pat. Nos. 5,840,693 and 5,607,918 by Ludwig Institute for Cancer Research and The University of Helsinki and in Olofsson et al., Proc. Natl. Acad. Sci. USA 1996 93:2576-2581). The nucleotide sequence for human VEGF-B is also found at GenBank Accession No. U48801. The entire disclosures of the International Patent Application PCT/US97/14696 (WO 98/07832), U.S. Pat. Nos. 5,840,693 and 5,607,918 are incorporated herein by reference.

The mouse and human genes for VEGF-B are almost identical, and both span about 4 kb of DNA. The genes are composed of seven exons and their exon-intron organization resembles that of the VEGF and PlGF genes (Grimmond et al., Genome Res. 1996 6:124-131); Olofsson et al., J. Biol. Chem. 1996 271:19310-19317); Townson et al., Biochem. Biophys. Res. Commun. 1996 220:922-928).

VEGF-C was isolated from conditioned media of the PC-3 prostate adenocarcinoma cell line (CRL1435) by screening for ability of the medium to produce tyrosine phosphorylation of the endothelial cell-specific receptor tyrosine kinase VEGFR-3 (Flt4), using cells transfected to express VEGFR-3. VEGF-C was purified using affinity chromatography with recombinant VEGFR-3, and was cloned from a PC-3 cDNA library. Its isolation and characteristics are described in detail in Joukov et al., EMBO J., 1996 15 290-298.

VEGF-D was isolated from a human breast cDNA library, commercially available from Clontech, by screening with an expressed sequence tag obtained from a human cDNA library designated “Soares Breast 3NbHBst” as a hybridization probe (Achen et al., Proc. Natl. Acad. Sci. USA, 1998 95:548553). Its isolation and characteristics are described in detail in International Patent Application No. PCT/US97/14696 (WO98/07832).

The VEGF-D gene is broadly expressed in the adult human, but is certainly not ubiquitously expressed. VEGF-D is strongly expressed in heart, lung and skeletal muscle. Intermediate levels of VEGF-D are expressed in spleen, ovary, small intestine and colon, and a lower expression occurs in kidney, pancreas, thymus, prostate and testis. No VEGF-D mRNA was detected in RNA from brain, placenta, liver or peripheral blood leukocytes.

PlGF was isolated from a term placenta cDNA library. Its isolation and characteristics are described in detail in Maglione et al., Proc. Natl. Acad. Sci. USA, 1991 88 9267-9271. Presently its biological function is not well understood.

VEGF2 was isolated from a highly tumorgenic, estrogen-independent human breast cancer cell line. While this molecule is stated to have about 22% homology to PDGF and 30% homology to VEGF, the method of isolation of the gene encoding VEGF2 is unclear, and no characterization of the biological activity is disclosed.

VEGF3 was isolated from a cDNA library derived from colon tissue. VEGF3 is stated to have about 36% identity and 66% similarity to VEGF. The method of isolation of the gene encoding VEGF3 is unclear and no characterization of the biological activity is disclosed.

Similarity between two proteins is determined by comparing the amino acid sequence and conserved amino acid substitutions of one of the proteins to the sequence of the second protein, whereas identity is determined without including the conserved amino acid substitutions.

PDGF/VEGF family members act primarily by binding to receptor tyrosine kinases. Five endothelial cell-specific receptor tyrosine kinases have been identified, namely VEGFR-1 (Flt-1), VEGFR-2 (KDR/Flk-1), VEGFR-3 (Flt4), Tie and Tek/Tie-2. All of these have the intrinsic tyrosine kinase activity which is necessary for signal transduction. The essential, specific role in vasculogenesis and angiogenesis of VEGFR-1, VEGFR-2, VEGFR-3, Tie and Tek/Tie-2 has been demonstrated by targeted mutations inactivating these receptors in mouse embryos.

The only receptor tyrosine kinases known to bind VEGFs are VEGFR-1, VEGFR-2 and VEGFR-3. VEGFR-1 and VEGFR-2 bind VEGF with high affinity, and VEGFR-1 also binds VEGF-B and PlGF. VEGF-C has been shown to be the ligand for VEGFR-3, and it also activates VEGFR-2 (Joukov et al., The EMBO Journal, 1996 15:290-298). VEGF-D binds to both VEGFR-2 and VEGFR-3. A ligand for Tek/Tie-2 has been described in International Patent Application No. PCT/US95/12935 (WO 96/11269) by Regeneron Pharmaceuticals, Inc. The ligand for Tie has not yet been identified.

Recently, a novel 130-135 kDa VEGF isoform specific receptor has been purified and cloned (Soker et al., Cell, 1998 92:735-745). The VEGF receptor was found to specifically bind the VEGF₁₆₅ isoform via the exon 7 encoded sequence, which shows weak affinity for heparin (Soker et al., Cell, 1998 92:735-745). Surprisingly, the receptor was shown to be identical to human neuropilin-1 (NP1), a receptor involved in early stage neuromorphogenesis. PIGF-2 also appears to interact with NP-1 (Migdal et al., J. Biol. Chem., 1998 273:22272-22278).

VEGFR-1, VEGFR-2 and VEGFR-3 are expressed differently by endothelial cells. Both VEGFR-1 and VEGFR-2 are expressed in blood vessel endothelia (Oelrichs et al., Oncogene, 1992 8:11-18; Kaipainen et al., J. Exp. Med., 1993 178:2077-2088; Dumont et al., Dev. Dyn., 1995 203:80-92; Fong et al., Dev. Dyn., 1996 207:1-10) and VEGFR-3 is mostly expressed in the lymphatic endothelium of adult tissues (Kaipainen et al., Proc. Natl. Acad. Sci. USA, 1995 9:3566-3570). VEGFR-3 is also expressed in the blood vasculature surrounding tumors.

Disruption of the VEGFR genes results in aberrant development of the vasculature leading to embryonic lethality around midgestation. Analysis of embryos carrying a completely inactivated VEGFR-1 gene suggests that this receptor is required for functional organization of the endothelium (Fong et al., Nature, 1995 376 66-70). However, deletion of the intracellular tyrosine kinase domain of VEGFR-1 generates viable mice with a normal vasculature (Hiratsuka et al., Proc. Natl. Acad. Sci. USA 1998 95:9349-9354). The reasons underlying these differences remain to be explained but suggest that receptor signalling via the tyrosine kinase is not required for the proper function of VEGFR-1. Analysis of homozygous mice with inactivated alleles of VEGFR-2 suggests that this receptor is required for endothelial cell proliferation, hematopoesis and vasculogenesis (Shalaby et al., Nature, 1995 376:62-66; Shalaby et al., Cell, 1997 89:981-990). Inactivation of VEGFR-3 results in cardiovascular failure due to abnormal organization of the large vessels (Dumont et al., Science, 1998 282:946949).

VEGFRs are expressed in many adult tissues, despite the apparent lack of constitutive angiogenesis. VEGFRs are however clearly upregulated in endothelial cells during development and in certain angiogenesis-associated/dependent pathological situations including tumor growth [see Dvorak et al., Amer. J. Pathol., 1995 146:1029-1039); Ferrara et al., Endocrine Rev., 1997 18:4-25)]. The phenotypes of VEGFR-1-deficient mice and VEGFR-2-deficient mice reveal an essential role for these receptors in blood vessel formation during development.

Although VEGFR-1 is mainly expressed in endothelial cells during development, it can also be found in hematopoetic precursor cells during early stages of embryogenesis (Fong et al., Nature, 1995 376:66-70). In adults, monocytes and macrophages also express this receptor (Barleon et al., Blood, 1996 87:3336-3343). In embryos, VEGFR-1 is expressed by most, if not all, vessels (Breier et al., Dev. Dyn., 1995 204:228-239; Fong et al., Dev. Dyn., 1996 207:1-10).

The receptor VEGFR-3 is widely expressed on endothelial cells during early embryonic development but as embryogenesis proceeds becomes restricted to venous endothelium and then to the lymphatic endothelium (Kaipainen et al., Cancer Res., 1994 54:6571-6577; Kaipainen et al., Proc. Natl. Acad. Sci. USA, 1995 92:3566-3570). VEGFR-3 is expressed on lymphatic endothelial cells in adult tissues. This receptor is essential for vascular development during embryogenesis. Targeted inactivation of both copies of the VEGFR-3 gene in mice resulted in defective blood vessel formation characterized by abnormally organized large vessels with defective lumens, leading to fluid accumulation in the pericardial cavity and cardiovascular failure at post-coital day 9.5.

On the basis of these findings it has been proposed that VEGFR-3 is required for the maturation of primary vascular networks into larger blood vessels. However, the role of VEGFR-3 in the development of the lymphatic vasculature could not be studied in these mice because the embryos died before the lymphatic system emerged. Nevertheless it is assumed that VEGFR-3 plays a role in development of the lymphatic vasculature and lymphangiogenesis given its specific expression in lymphatic endothelial cells during embryogenesis and adult life. This is supported by the finding that ectopic expression of VEGF-C, a ligand for VEGFR-3, in the skin of transgenic mice, resulted in lymphatic endothelial cell proliferation and vessel enlargement in the dermis. Furthermore this suggests that VEGF-C may have a primary function in lymphatic endothelium, and a secondary function in angiogenesis and permeability regulation which is shared with VEGF (Joukov et al., EMBO J., 1996 15:290298).

VEGFR-1-deficient mice die in utero at mid-gestation due to inefficient assembly of endothelial cells into blood vessels, resulting in the formation of abnormal vascular channels [Fong et al., Nature, 1995 376:66-70)]. Analysis of embryos carrying a completely inactivated VEGFR-1 gene suggests that this receptor is required for functional organization of the endothelium (Fong et al., Nature, 1995 376:66-70). However, deletion of the intracellular tyrosine kinase domain of VEGFR-1 generates viable mice with a normal vasculature (Hiratsuka et al., Proc. Natl. Acad. Sci. USA, 1998 95:9349-9354). The reasons underlying these differences remain to be explained but suggest that receptor signalling via the tyrosine kinase is not required for the proper function of VEGFR-1.

VEGFR-2-deficient mice die in utero between 8.5 and 9.5 days post-coitum, and in contrast to VEGFR-1, this appears to be due to abortive development of endothelial cell precursors (Shalaby et al., Nature 1995 376:62-66); Shalaby et al., Cell, 1997 89:981-990), suggesting that this receptor is required for endothelial cell proliferation, hematopoesis and vasculogenesis. The importance of VEGFR-2 in tumor angiogenesis has also been clearly demonstrated by using a dominant-negative approach (Millauer et al., Nature, 1994 367:576-579); Millauer et al., Cancer Res. 1996 56:1615-1620).

The phenotype of VEGFR-3-deficient mice has been reported in Dumont, et al., Cardiovascular Failure in Mouse Embryos Deficient in VEGF Receptor-3, Science, 1998 282:946-949). VEGFR-3 deficient mice die in utero between 12 and 14 days of gestation due to defective blood vessel development. On the basis of these findings it has been proposed that VEGFR-3 is required for the maturation of primary vascular networks into larger blood vessels. However, the role of VEGFR-3 in the development of the lymphatic vasculature could not be studied in these mice because the embryos died before the lymphatic system emerged. Nevertheless it is assumed that VEGFR-3 plays a role in development of the lymphatic vasculature and lymphangiogenesis given its specific expression in lymphatic endothelial cells during embryogenesis and adult life.

This is supported by the finding that ectopic expression of VEGF-C, a ligand for VEGFR-3, in the skin of transgenic mice, resulted in lymphatic endothelial cell proliferation and vessel enlargement in the dermis (Makinen et al., Nature Med, 2001 7:199-205). Furthermore this suggests that VEGF-C may have a primary function in lymphatic endothelium, and a secondary function in angiogenesis and permeability regulation which is shared with VEGF (Joukov et al., EMBO J., 1996 15: 290-298).

Some inhibitors of the VEGF/VEGF-receptor system have been shown to prevent tumor growth via an anti-angiogenic mechanism; see Kim et al., Nature, 1993 362:841-844 and Saleh et al., Cancer Res., 1996 56:393-401.

As mentioned above, the VEGF family of growth factors are members of the PDGF family. PDGF plays a important role in the growth and/or motility of connective tissue cells, fibroblasts, myofibroblasts and glial cells (Heldin et al., “Structure of platelet-derived growth factor: Implications for functional properties”, Growth Factor, 1993 8:245-252). In adults, PDGF stimulates wound healing (Robson et al., Lancet, 1992 339:23-25). Structurally, PDGF isoforms are disulfide-bonded dimers of homologous A- and B-polypeptide chains, arranged as homodimers (PDGF-AA and PDGF-BB) or a heterodimer (PDGF-AB).

PDGF isoforms exert their effects on target cells by binding to two structurally related receptor tyrosine kinases (RTKs). The alpha-receptor binds both the A- and B-chains of PDGF, whereas the beta-receptor binds only the B-chain. These two receptors are expressed by many in vitro grown cell lines, and are mainly expressed by mesenchymal cells in vivo. The PDGFs regulate cell proliferation, cell survival and chemotaxis of many cell types in vitro (reviewed in Heldin et al., Biochim Biophys Acta., 1998 1378:F79-113). In vivo, they exert their effects in a paracrine mode since they often are expressed in epithelial (PDGF-A) or endothelial cells (PDGF-B) in close apposition to the PDGFR expressing mesenchyme.

In tumor cells and in cell lines grown in vitro, coexpression of the PDGFs and the receptors generate autocrine loops which are important for cellular transformation (Betsholtz et al., Cell, 1984 39:447-57; Keating et al., J. R. Coll Surg Edinb., 1990 35:172-4). Overexpression of the PDGFs have been observed in several pathological conditions, including maligancies, arteriosclerosis, and fibroproliferative diseases (reviewed in Heldin et al., The Molecular and Cellular Biology of Wound Repair, New York: Plenum Press, 1996, 249-273).

The importance of the PDGFs as regulators of cell proliferation and survival are well illustrated by recent gene targeting studies in mice that have shown distinct physiological roles for the PDGFs and their receptors despite the overlapping ligand specificities of the PDGFRs. Homozygous null mutations for either of the two PDGF ligands or the receptors are lethal. Approximately 50% of the homozygous PDGF-A deficient mice have an early lethal phenotype, while the surviving animals have a complex postnatal phenotype with lung emphysema due to improper alveolar septum formation because of a lack of alveolar myofibroblasts (Boström et al., Cell, 1996 85:863-873). The PDGF-A deficient mice also have a dermal phenotype characterized by thin dermis, misshapen hair follicles and thin hair (Karlsson et al., Development, 1999 126:2611-2).

PDGF-A is also required for normal development of oligodendrocytes and subsequent myelination of the central nervous system (Fruttiger et al., Development, 1999 126:457-67). The phenotype of PDGFR-alpha deficient mice is more severe with early embryonic death at E10, incomplete cephalic closure, impaired neural crest development, cardiovascular defects, skeletal defects, and odemas [Soriano et al., Development, 1997 124:2691-70).

The PDGF-B and PDGFR-beta deficient mice develop similar phenotypes that are characterized by renal, hematological and cardiovascular abnormalities (Levéen et al., Genes Dev., 1994 8:1875-1887; Soriano et al., Genes Dev., 1994 8:1888-96; Lindahl et al., Science, 1997 277:242-5; Lindahl, Development, 1998 125:3313-2), where the renal and cardiovascular defects, at least in part, are due to the lack of proper recruitment of mural cells (vascular smooth muscle cells, pericytes or mesangial cells) to blood vessels (Levéen et al., Genes Dev., 1994 8:1875-1887; Lindahl et al., Science, 1997 277:242-5; Lindahl et al., Development, 1998 125:3313-2).

PDGF-C and PDGF-D have only recently been discovered (Li, X., et al, PDGF-C is a New Protease Activated Ligand for the PDGF alpha Receptor, Nat Cell Ciol., 2000 2(5):302-309; Bergsten, E., et al., PDGF-D is a Specific, Protease-Activated Ligand for the PDGF beta Receptor, Nat Cell Biol., 2001 3(5):512-516). PDGF-C is produced as a 95 kD homodimer, PDGF-CC, and needs to be proteolytically activated to bind and activate PDGF receptor alpha²⁴. PDGF-C displays a unique protein structure by processing a so-called CUB domain, which has high homology to the same domain in the neutropilin 1 (NP-1) gene (Hamada, T., et al., A Novel Gene Derived from Developing Spinal Cords, SCDGF, is a Unique Member of the PDGF/VEGF Family, FEBS Lett, 2000 475(2):97-102)

PDGF-C is widely expressed in mesenchymal precursor cells, epithelial cells, muscular tissues, vascular smooth muscle cells of the larger arteries, spinal cord and developing skeleton system, supporting a role in organogenesis (Tsai, Y. J., et al., Identification of a Novel Platelet-Derived Growth Factor-Like Gene, Fallotein, in the Human Reproductive Tract, Biochim Biophys Acta, 2000 1492(1):196-202; Ding, H. et al., The Mouse PDGFC Gene: Dynanic Expression in Embryonic Tissues During Organogenesis, Mech Dev, 2000 96(2):209-213).

Over expression of PDGF-C in the heart leads to cardiomyocyte hypertrophy and fibrosis, suggesting a requirement for a fine-tuned control of PDGF-C expression in the heart under normal conditions. PDGF-C has also recently been shown to be a potent angiogenic factor in both the mouse cornea and the chorion allantoic membrane (CAM) assays by stimulating the formation of long and slender vessels, much like those induced by FGF-2. PDGF-C promoted SMC growth in aortic ring outgrowth assay and wound healing (Gilbertson, D. G., et al., Platelet-Derived Growth Factor C (PDGF-C) a Novel Growth Factor that Binds to PDGF (alpha) and (beta) Receptor, J Biol Chem, 2001 276:27406-27414). PDGF-C has recently been shown to be an EWS/FLI induced transforming growth factor (Zwerner, J. P. and May, W. A., PDGF-C is an EWS/FLI Induced Transforming Growth Factor in Ewing Family Tumors, Oncogene, 2001 20(5):626-633), and expressed in many cell lines (Uutela, M., et al., Chromosomal Location, Exon Structure, and Vascular Expression Patterns of the Human PDGFC and PDGFD Genes, Circulation, 2001 103(18):2242-2247), indicating a role in tumorigenesis.

PDGF-D is produced as a latent homodimer similar to PDGF-C and binds and activates PDGF-R beta upon proteolytic activation. It is highly expressed in the heart, pancreas, ovary, and to a less extent, in most other organs. The biological role of PDGF-D is not yet exhaustively explained.

Acute and chronic myocardial ischemia are the leading causes of morbidity and mortality in the industralized society caused by coronary thrombosis (Varbella, F., et al., Subacute Left Ventricular Free-Wall Rupture in Early Course of Acute Myocardial Infarction. Climical Report of Two Cases and Review of the Literature, G Ital Cardiol, 1999 29(2):163-170). Immediately after heart infarction, oxygen starvation causes cell death of the infarcted area, followed by hypertrophy of the remaining viable cardiomyocytes to compensate the need of a normal contractile capacity (Heymans, S., et al., Inhibition of Plasminogen Activators or Matrix Metalloproteinases Prevents Cardiac Rupture but Impairs Therapeutic Angiogenesis and Causes Cardiac Failure, Nature Medicine, 1999 5(10):1135-1142).

Prompt post-infarction reperfusion of the infarcted leftventricular wall may significantly reduce the early mortality and subsequent heart failure by preventing apoptosis of the hypertrophied viable myocytes and pathological ventricular remodelling (Dalrymple-Hay, M. J., et al., Postinfarction Ventricular Septal Rupture: the Wessex Experience, Semin Thorac Cardiovasc Surg, 1998 10(2):111-116). Despite the advances in clinical treatment and prevention, however, insufficient post-infarction revascularization remains to be the major cause of the death of the otherwise viable myocardium and leads to progressive infarct extension and fibrous replacement, and ultimately heart failure. Therefore, therapeutic agents promoting post-infarction revascularization with minimal toxicity are still needed.

More than 750,000 people in the United States suffer from critical limb ischemia (CLI), a disease manifested by sharply diminished blood flow to the legs. CLI leads to amputation for 200,000 people per year in the Untied States. Up to 10 million people in the United States suffer from severe leg pain (claudication) and non-healing ulcers (peripheral vascular disease), both of which may ultimately lead to CLI. Peripheral vascular disease (PVD) is linked to cardiovascular disease in general, and is often associated with diabetes, lifestyle and aging. There are no drugs currently approved for the treatment of CLI. Thus, there is also a need for new methods for treating limb ischemia.

For therapeutic revascularization of ischemic tissues to succeed, the newly formed vessels must be mature, durable and functional. This implies not only that new endothelium-lined vessels must sprout (“angiogenesis”), but also that these nascent vessels become covered by perivascular smooth muscle cells and/or fibroblasts (“arteriogenesis”)—processes that require an involvement of both vascular progenitors and differentiated cells of multiple vascular cell types. While angio/arteriogenesis are easily deregulated by inactivation of candidate genes (Carmeliet et al., 1996, Nature 380, 435-9; Hellström et al. 2001, J Cell Biol 153, 543-53), stimulating these processes in a functionally relevant manner has proven to be a much greater challenge than anticipated. Apart from the existing candidates, new molecules may be required to achieve this goal—preferentially those having pleiotropic activities on both vascular progenitors and differentiated vascular cells of both endothelial and smooth muscle cell lineages.

It is generally accepted that PDGF-AA and -BB are the major mitogen and chemoatractant for cells of mesenchymal origion, but have no, or little effect on cells of endothelial lineage, although both PDGFR-α and -β are expressed on endothelial cells (Edelberg et al., 1998, Journal of Clinical Investigation 102: 837-43; Smits et al., 1989, Growth Factors 2: 1-8; Bar et al., 1989, Endocrinology 124: 1841-8; Beitz et al., 1991, Proc Natl Acad Sci USA 88, 2021-5; Marx et al., 1994, J. Clin. Invest. 93:131-9; and Shinbrot, et al., 1994, Dev. Dyn. 199: 169175) In line with this, the angiogenic/arteriogenic activity of PDGF-AA and -BB still remains an issue of debate after more than twenty years of investigation. PDGF-BB and -AB have been shown to be involved in the stabilization/maturation of newly formed vessels (Isner, Nature, 2002 415:234-9; Vale et al., J Interv Cardiol, 2001 14:511-28; Heldin et al., Physiol Rev, 1999 79:1283-1316; Betsholtz et al., Bioessays, 2001 23:494-507). Other data however, showed that PDGF-BB and PDGF-AA inhibited bFGF-induced angiogenesis in vivo via PDGFR-α signaling (Edelberg et al., Journal of Clinical Investigation, 1998 102:837-43). Thus, the angiogenic/arteriogenic activity of the PDGFs, especially when signaling through PDGFR-α, has been controversial and enigmatic.

As discussed above, during development, PDGF-C is expressed in muscle progenitor cells and differentiated smooth muscle cells in most organs, including the heart, lung and kidney (Aase et al., Mech Dev, 2002 110:187-191). In adulthood, PDGF-C is widely expressed in most organs, with the highest expression level in the heart and kidney (Li et al., Nat Cell Biol, 2000 2:302-309). Activated PDGF-CC is capable of binding and activating its receptor, PDGFR-α. In cells co-expressing both PDGFR-α and -β, PDGF-CC may also activate the PDGFR-α/β heterodimer, but not the PDGFR-P/p homodimer (Cao et al., Faseb J, 2002 16:1575-83; Gilbertson et al., J Biol Chem, 2001 10:10). PDGF-CC is capable of promoting physiological vascular development in the embryo and in healing wounds, and angiogenesis in avascular tissues (Cao et al., Faseb J, 2002 16:1575-83; Gilbertson et al., J Biol Chem, 2001 10:10), but prior to the present invention, it was uncertain whether PDGF-CC could effectively stimulate the growth and maturation of new vessels in the ischemic myocardium or limb. In addition, the cellular mechanisms underlying its angiogenic activities remain undetermined. In particular, although PDGF-CC is a known potent mitogen for fibroblast and vascular smooth muscle cells (Li et al., Nat Cell Biol, 2000 2:302309; Gilbertson et al., J Biol Chem, 2001 10:10; Uutela et al., Circulation, 2001 103:2242-7), prior to the present invention, it was not known whether it promotes the maturation of newly formed vessels (arteriogenesis)—a prerequisite to build functional vessels. Moreover, it was unknown whether PDGF-CC affects the mobilization or differentiation of vascular progenitors to sites of active vessel growth in the adult (a process termed “adult vasculogenesis”); and if so, whether it has any selective effect on endothelial or smooth muscle cell fate commitment. Furthermore, it was also unknown whether PDGF-CC affects endothelial cells directly.

The building of new stable and functional vessels relies on a concerted action of vascular progenitors and their differentiated endothelial and smooth muscle cells. Therapeutic angiogenesis may thus require co-administration of molecules like VEGF and PDGF-BB, which primarily affect the endothelial or smooth muscle cell lineage, respectively. Alternatively, molecules with pleiotropic effects on both lineages would be attractive, but only a few have been identified thus far. Therefore, there is a need for additional molecules with the desired pleiotropic effects, pharmaceutical compositions and methods of use thereof.

### SUMMARY OF THE INVENTION

Unitary activity on a single type of cell leading to unfunctional capillaries, or harmful side effects involving edema or angioma-genesis, is often the central problem for therapeutic angio/arteriogenic agents under trial. Molecules with pleiotrotic activities affecting multiple vascular cells or stages of angiogenesis and/or arteriogenesis, but with minimal side effects, would be attractive means to treat tissue ischemia. Using different in vivo and in vitro models, the present invention showed, for the first time, that the pleiotropic activities of PDGF-CC in affecting both vascular progenitors and differentiated cells of both EC and SMC lineages, together with its safety profile (lack of hemangioma-genesis, edema or fibrosis), and restricted activity in ischemic conditions, and its beneficial effects on muscle regeneration, form the basis for novel strategies to treat patients with heart and limb ischemia. There are considerable potential advantages of choosing such molecules, including mobilizing multiple vascular cells and molecules needed to build functional vessels by a single delivery of one effector molecule, and the simultaneous regulation of the complex cascade of angio/arteriogenesis with one therapeutic intervention. The present invention provide new therapeutic agents to cultivate functional vessels with more physiological functional properties in treating tissue ischemia.

The present inventors showed that PDGF-CC has both angiogenic and arteriogenic activities. In particular, PDGF-CC stimulates the growth of functional vessels in both cardiac and limb ischemia, and displays pleiotropic effects on vascular progenitor and differentiated cells of both endothelial cell (EC) and smooth muscle cell (SMC) lineages. This activity of PDGF-CC is surprising and intriguing, when considering that the traditional function of PDGFs in adults, after more than twenty years of research, has been mainly confined to stimulating connective tissue production and mesenchymal cell growth (Heldin et al., Physiol Rev, 1999 79:1283-1316), and that PDGFR-α signaling has previously been considered to be poorly angiogenic—or even to suppress vascular growth (De Marchis et al., Blood, 2002 99:2045-2053; Palumbo et al., Arterioscler Thromb Vasc Biol, 2002 22:405-11). These data thus reveal unprecedented functional properties of PDGF-CC. Accordingly, novel therapeutic methods and compositions are also provided by the present invention.

Pleiotropic Activity of PDGF-CC on Vascular Progenitors and Differentiated Cells of both EC and SMC-Oriented Lineages

The angio/arteriogenic effect of PDGF-CC involves several mechanisms, including mobilization and differentiation of vascular progenitors, chemotactic effect on differentiated ECs and SMCs, proliferation and migration of perivascular cells, and upregulation of VEGF expression. Thus, in contrast to VEGF or PDGF-AA and -BB, whose vascular effects are largely restricted to EC or SMC/fibroblast cells, respectively, the effect of PDGF-CC on the vasculature is more pleiotropic and thus allows a more synchronized, universal action of the different cell types, needed to build functional blood vessels. It is now amply documented that adult bone marrow-derived progenitor cells can contribute to the revascularization and, thereby, facilitate the regeneration and functional recovery of the ischemic limb and heart (Bianco et al., Nature, 2001 414:118-21; Kocher et al., Nat Med, 2001 7:430-6; Tateishi-Yuyama et al., Lancet, 2002 360:427-35; Strauer et al., Circulation, 2002 106:1913-8). Prior to the present invention, however, the signals that trigger their mobilization and induce their differentiation into more specialized vascular cells were unknown. The present inventors showed that PDGF-CC mobilizes endothelial progenitor cells (EPCs) within the first two days and up to 5 days after tissue ischemia. This is precisely the time window when new blood vessels start to grow in ischemic tissues. Thus the present data demonstrate that PDGF-CC can be employed to mobilize EPCs at a time of active revascularization of ischemic tissues. It is also possible that endogenous PDGF-CC is also involved in the recruitment of EPCs.

The present invention further demonstrate that PDGF-CC promoted AC133⁺CD34⁺progenitors to differentiate into cell types with markers characteristic of either endothelial cells (CD144, CD31) or SMCs. VEGF, instead, only promoted these progenitors to differentiate into endothelial cells. PDGF-CC initially promoted the differentiation of both lineages, but subsequently, after prolonged treatment, favored the differentiation of smooth muscle cells. There are two possibilities, one is that PDGF-CC stimulates the transdifferentiation of endothelial to smooth muscle cells, the other is that it enhances the selection of smooth muscle cells starting from a single population of a common vascular progenitor or from a mixed population of endothelial and smooth muscle cell progenitors.

Importantly, however, a possible implication of the findings presented in this disclosure is that if only VEGF is present, bone marrow-derived progenitors will preferentially contribute to the formation of endothelial capillaries. In comparison, when PDGF-CC is present, it might favor the differentiation of bone marrow progenitors into both endothelial and smooth muscle cells and, thereby, not only promote the formation of endothelial capillaries but, additionally, also the stabilization of these nascent vessels with smooth muscle cells. The activity of PDGF-CC is, however, not restricted to vascular progenitors only, as this growth factor also stimulated the migration of differentiated ECs and the migration and proliferation of SMCs—when studied both as isolated cultured cells or in the aortic ring assay.

Thus, by mobilizing vascular progenitors, by promoting their differentiation into both endothelial and smooth muscle cells, and by stimulating these differentiated vascular cells, PDGF-CC is shown to be able to orchestrate the complex process of building mature, durable and functional vessels. As further support, PDGF-CC was found to increase the perfusion of the ischemic myocardium by revascularizing the myocardium not only with SMC-covered coronary vessels (providing bulk flow) but also with endothelial-lined capillaries (distributing the flow to the individual cardiomyocytes). In the ischemic limb, PDGF-CC was also found to stimulate both angiogenesis and arteriogenesis. Moreover, the observation that PDGF-CC also enhanced muscle regeneration in areas of active revascularization further underscores that the new vessels were functional and perfused. The pleiotropic activity of PDGF-CC may also explain why no side effects of hemangioma-genesis and edema formation after PDGF-CC treatment were observed, but such side effects have been observed after VEGF administration (Carmeliet, Nat Med, 2000 6:1102-3).

Few growth factors have such a broad spectrum of activities as PDGF-CC: VEGF has more restricted effects on endothelial progenitors and differentiated cells, while PDGF-AA, PDGF-BB and TGF-β predominantly affect fibroblasts and SMCs (Battegay et al., Cell, 1990 63:515-24; Janat et al., J Cell Physiol, 1992 150:232-42; Stouffer et al., J Clin Invest, 1994 93:2048-55). PDGF-CC resembles, to a certain extent, bFGF and PlGF, which also affect progenitors and differentiated ECs and SMCs (Luttun et al., Nat Med, 2002 1:1; Hattori et al., Nat Med, 2002 1:1). However, none of these molecules has been documented to induce the expression of SMC genes in adult bone marrow-derived progenitors, and very few molecules have been discovered to regulate the differentiation and function of SMC progenitors derived from adult BM stem cells (Hirschi et al., Gene Ther, 2002 9:648-52). PDGF-BB stimulates embryonic vascular progenitors to acquire a SMC-phenotype (Carmeliet, Nature, 2000 408:43-45; Yamashita et al., Nature, 2000 408:92-6), but is unknown to have similar effects on adult bone marrow-derived progenitors. Thus, PDGF-CC has unique properties, which distinguishes it from previous vascular signals.

PDGFR-α: a Novel Function for an Old Receptor?

Even though discovered now more than twenty years ago (Heldin et al., Proceedings of the National Academy of Sciences of the United States of America, 1979 76:3722-6; Heldin et al., Proceedings of the National Academy of Sciences of the United States of America, 1981 78:3664-8), the role of PDGF-AA and its receptor PDGFR-α in vascular growth still remains enigmatic. Although intensive studies have established that PDGF-AA is among the most potent stimuli of mesenchymal cell migration, it does not or minimally stimulate—and, in certain conditions, even inhibits—EC migration (De Marchis et al., Blood, 2002 99:2045-2053). Moreover, PDGFR-α has been shown to antagonize the PDGFR-β-induced SMC migration (Yu et al., Biochem Biophys Res Commun, 2001 282:697-700), and neutralizing antibodies against PDGF-AA enhance SMC migration (Palumbo et al., Arterioscler Thromb Vasc Biol, 2002 22:405-11). The present inventors showed that PDGF-CC affected both ECs and SMCs, while PDGF-AA only affected SMCs and minimally affected endothelial outgrowth in the aortic ring assay—even though both ligands bind PDGFR-α. This unique chemotactic activity of PDGF-CC on ECs is further supported by a recent finding of impaired EC migration in the PDGFR-α deficient gonad, where PDGF-CC but not likely PDGF-AA or -BB—was considered to be the effector molecule involved (Brennen et al., 2003, Genes Dev. 17:800-810). The findings may imply a unique signaling pathway for each ligand. The distinct activities of PDGF-AA and -CC may further help to explain why loss of PDGFR-α causes a more severe phenotype than that caused by elimination of PDGF-A gene alone (Betsholtz et al., Bioessays, 2001 23:494-507; Li et al., Nat Cell Biol, 2000 2:302-309).

PDGF-CC Stimulates Post-Ischemic Vascular and Muscle Regeneration

The present inventors showed that PDGF-CC treatment mobilized endothelial progenitors and increased the vessel density and blood perfusion in the ischemic heart and limb, but did not affect quiescent vessels in other organs. Although PDGF-CC enlarged the second-generation side branches of the collateral vessels in the adductor muscle, this growth factor has, overall, a less dramatic effect on the remodeling of the preexisting collaterals in the upper limb region after femoral artery ligation than, for instance, bFGF, PlGF or GM-CSF (Luttun et al., Nat Med, 2002 1:1; Chleboun et al., Biochem Biophys Res Commun, 1992 185:510-6; Seiler et al., Circulation, 2001 104:2012-7). However, the molecular and cellular mechanisms of the growth of collateral vessels are quite distinct from those determining the formation of new capillaries and their maturation by coverage with smooth muscle cells. In particular, not ischemia but shear stress-induced recruitment of monocytes/macrophages is well known to play a critical role in initiating collateral growth in the upper hindlimb (Schaper et al. Circ. Res, 1996) and PDGF-CC does not affect their recruitment. Since only the lower, but not the upper limb is ischemic after femoral artery ligation, PDGF-C seems to be involved more in ischemia-dependent angiogenesis than in the shear stress-induced collateral remodeling.

Given the general expression pattern of PDGFR-α on most types of cells (Heldin et al., Physiol Rev, 1999 79:1283-1316) and the fact that PDGF-AA stimulated oligodendrocyte precursor proliferation (Baron et al., Embo J, 2002 21:1957-66), PDGF-CC may affect additional cell types. For example, the present disclosure showed that muscle regeneration after femoral artery ligation was improved by PDGF-CC, especially in regions where vascular regeneration was also maximal. Although this is could be an indirect effect of revascularization, it may also be a direct effect by PDGF-CC on muscle-derived stem cells (Deasy et al., Curr Opin Mol Ther, 2002 4:382-9).

The invention accordingly generally provides compositions and methods for the treatment of conditions associated with PDGF-C over or under expression. According to one embodiment of the present invention, a method of promoting revascularization is provided, which comprises administering a revascularization promoting amount of a pharmaceutical composition according to the present invention. This treatment method may be used for, inter alia, promoting revascularization in post-infarction or other ischemic tissue or promoting revascularization with small vessels. According to another embodiment of the present invention, a method of increasing vessel density is provided, comprising administering an effective vessel density increasing amount of the pharmaceutical composition of the present invention.

In one preferred embodiment, a pharmaceutical composition for modulating vasculogenesis or arteriogenesis or angiogenesis, comprises a pharmaceutically effective amount of a polypeptide having at least 85% sequence identity with the sequence of SEQ ID NO:40. Preferably, the pharmaceutical composition of the present invention is for enhancing post-ischemic revascularization in the heart and limb. Also provided are methods of treating ischemic diseases, especially cardiovascular ischemia and limb ischemia. Aslo provided are methods for neoangiogenesis required in the ischemic brain following stroke.

In another embodiment, the pharmaceutical composition may further comprise one or more of PDGF-A, PDGF-B, PDGF-D, VEGF, VEGF-B, VEGF-C, VEGF-D, PlGF and/or heparin. The pharmaceutical composition may further comprise a pharmaceutical carrier or diluent.

In a further embodiment, the pharmaceutical composition comprises a pharmaceutically effective amount of an expression vector which expresses a polypeptide having at least 85% sequence identity with the sequence of SEQ ID NO:40.

A pharmaceutical composition for modulating vasculogenesis, arteriogenesis or angiogenesis according to the present invention preferably comprises a pharmaceutically effective amount of a polypeptide dimer comprising a polypeptide having at least 85% sequence identity with the sequence of SEQ ID NO:40. The dimer may a heterodimer comprising an active monomer of VEGF, VEGF-B, VEGF-C, VEGF-D, PDGF-C, PDGF-A, PDGF-B, PDGF-D or PlGF and an active monomer of PDGF-C. In a particularly preferred embodiment, the pharmaceutical composition comprises a homodimer of activated PDGF-C, which is designated hereinafter PDGF-CC.

The present invention also provides for a method for modulating vasculogenesis or arteriogenesis or angiogenesis or both, said method comprising administering a subject in need thereof a pharmaceutically effective amount of a polypeptide having at least 85% sequence identity with the sequence of SEQ ID NO:40. Preferably, the method is for treating ischemia, especially myocardial ischemia, and limb ischemia. In particular, the method is for treating chronic myocardial ischemia, or critical limb ischemia. In a preferred embodiment, vasculogenesis or arteriogenesis or angiogenesis, or both, in the subject are increased, especially in ischemic tissue of the subject.

In one embodiment, the polypeptide is administered into ischemic tissue of the subject, such as via injection or a subcutaneous minipump.

The present invention further provides a method for modulating vasculogenesis or arteriogenesis or angiogenesis or both, said method comprising administering a subject in need thereof a pharmaceutically effective amount of a polynucleotide encoding a polypeptide having at least 85% sequence identity with the sequence of SEQ ID NO:40. Preferably, the polynucleotide is an expression vector suitable for gene therapy. Viral vectors, e.g. adeno-associated virus (AAV) derived vectors, adenoviral vectors, retroviral and lentiviral vectors may be used. These vectors are well known in the art.

Also provided is a method for promoting differentiation in vivo of bone marrow cells into smooth muscle cells or endothelial cells, or both, the method comprising administering to a subject in need thereof an effective amount of a polypeptide having at least 85% sequence identity with the sequence of SEQ ID NO:40, or a polynucleotide encoding said polypeptide. Still further provided is a method for inducing smooth muscle cell gene expression, such as the expression of SMA, in vivo in an adult bone marrow cell, and a method for stimulating both angiogenesis and arteriogenesis in ischemic tissue of a subject in need thereof.

In a further embodiment, a method for improving abnormal cardiac function in a mammal is provided. The method comprises: a) injecting into heart muscle of said mammal a DNA encoding a polypeptide having at least 85% sequence identity with the sequence of SEQ ID NO:40, and b) obtaining expression of said polypeptide in said heart muscle in an amount that increases vasculogenesis or arteriogenesis or angiogenesis within the heart muscle, thereby improving cardiac function.

While not willing to be limited by any particular theory or hypothesis, it is believed that PDGF-CC effects is pleotropic activity by a concerted action on the vascular progenitor and mature cells of both endothelial and smooth muscle cell/fibroblast lineages. The instant disclosure will show that PDGF-CC mobilizes endothelial progenitor cells, induces differentiation of bone marrow cells into endothelial cells, stimulates migration of endothelial cells, and upregulates VEGF expression. Moreover, the present inventors have shown that PDGF-CC induces the differentiation of bone marrow cells into smooth muscle cells and stimulated their growth and migration during vessel sprouting.

As used in this application, percent sequence identity is determined by using the alignment tool of “MEGALIGN” from the Lasergene package (DNASTAR, Ltd. Abacus House, Manor Road, West Ealing, London W130AS United Kingdom) and using its preset conditions. The alignment is then refined manually, and the number of identities are estimated in the regions available for a comparison.

As used herein, the term “PDGF-C” collectively refers to the polypeptides of FIG. 2 (SEQ ID NO:3), FIG. 4 (SEQ ID NO:5) or FIG. 6 (SEQ ID NO:7), and fragments or analogs thereof which have the biological activity of PDGF-C as defined above, including SEQ ID NO: 40, the active core domain of PDGF-C and to a polynucleotide which can code for PDGF-C, or a fragment or analog thereof having the biological activity of PDGF-C. The polynucleotide can be naked and/or in a vector or liposome.

In another preferred aspect, the invention provides a polypeptide possessing an amino acid sequence: PXCLLVXRCGGXCXCC (SEQ ID NO:1) which is unique to PDGF-C and differs from the other members of the PDGF/VEGF family of growth factors because of the insertion of the three amino acid residues (NCA) between the third and fourth cysteines (see FIG. 9-SEQ ID NOs:8-17).

Polypeptides comprising conservative substitutions, insertions, or deletions, but which still retain the biological activity of PDGF-C are clearly to be understood to be within the scope of the invention. Persons skilled in the art will be well aware of methods which can readily be used to generate such polypeptides, for example the use of site-directed mutagenesis, or specific enzymatic cleavage and ligation. The skilled person will also be aware that peptidomimetic compounds or compounds in which one or more amino acid residues are replaced by a non-naturally occurring amino acid or an amino acid analog may retain the required aspects of the biological activity of PDGF-C. Such compounds can readily be made and tested by methods known in the art, and are also within the scope of the invention.

In addition, possible variant forms of the PDGF-C polypeptide which may result from alternative splicing, as are known to occur with VEGF and VEGF-B, and naturally-occurring allelic variants of the nucleic acid sequence encoding PDGF-C are encompassed within the scope of the invention. Allelic variants are well known in the art, and represent alternative forms or a nucleic acid sequence which comprise substitution, deletion or addition of one or more nucleotides, but which do not result in any substantial functional alteration of the encoded polypeptide.

Such variant forms of PDGF-C can be prepared by targeting nonessential regions of the PDGF-C polypeptide for modification. These nonessential regions are expected to fall outside the strongly-conserved regions indicated in FIG. 9 (SEQ ID NOs:8-17). In particular, the growth factors of the PDGF family, including VEGF, are dimeric, and VEGF, VEGF-B, VEGF-C, VEGF-D, PlGF, PDGF-A and PDGF-B show complete conservation of eight cysteine residues in the N-terminal domains, i.e. the PDGF/VEGF-like domains (Olofsson et al., Proc. Natl. Acad. Sci. USA, 1996 93:2576-2581; Joukov et al., EMBO J., 1996 15:290-298). These cysteines are thought to be involved in intra- and inter-molecular disulfide bonding. In addition there are further strongly, but not completely, conserved cysteine residues in the C-terminal domains. Loops 1, 2 and 3 of each subunit, which are formed by intra-molecular disulfide bonding, are involved in binding to the receptors for the PDGF/VEGF family of growth factors (Andersson et al., Growth Factors, 1995 12:159-164).

Persons skilled in the art thus are well aware that these cysteine residues should be preserved in any proposed variant form, and that the active sites present in loops 1, 2 and 3 also should be preserved. However, other regions of the molecule can be expected to be of lesser importance for biological function, and therefore offer suitable targets for modification. Modified polypeptides can readily be tested for their ability to show the biological activity of PDGF-C by routine activity assay procedures such as the fibroblast proliferation assay of Example 6.

It is contemplated that some modified PDGF-C polypeptides will have the ability to bind to PDGF-C receptors on cells including, but not limited to, endothelial cells, connective tissue cells, myofibroblasts and/or glial cells, but will be unable to stimulate cell proliferation, differentiation, migration, motility or survival or to induce vascular proliferation, connective tissue development or wound healing. These modified polypeptides are expected to be able to act as competitive or non-competitive inhibitors of the PDGF-C polypeptides and growth factors of the PDGF/VEGF family, and to be useful in situations where prevention or reduction of the PDGF-C polypeptide or PDGF/VEGF family growth factor action is desirable.

Thus such receptor-binding but non-mitogenic, non-differentiation inducing, non-migration inducing, non-motility inducing, non-survival promoting, non-connective tissue development promoting, non-wound healing or non-vascular proliferation inducing variants of the PDGF-C polypeptide are also within the scope of the invention, and are referred to herein as “receptor-binding but otherwise inactive variant”. Because PDGF-C forms a dimer in order to activate its only known receptor, it is contemplated that one monomer comprises the receptor-binding but otherwise inactive variant modified PDGF-C polypeptide and a second monomer comprises a wild-type PDGF-C or a wild-type growth factor of the PDGF/VEGF family. These dimers can bind to its corresponding receptor but cannot induce downstream signaling.

It is also contemplated that there are other modified PDGF-C polypeptides that can prevent binding of a wild-type PDGF-C or a wild-type growth factor of the PDGF/VEGF family to its corresponding receptor on cells including, but not limited to, endothelial cells, connective tissue cells (such as fibroblasts), myofibroblasts and/or glial cells. Thus these dimers will be unable to stimulate endothelial cell proliferation, differentiation, migration, survival, or induce vascular permeability, and/or stimulate proliferation and/or differentiation and/or motility of connective tissue cells, myofibroblasts or glial cells. These modified polypeptides are expected to be able to act as competitive or non-competitive inhibitors of the PDGF-C growth factor or a growth factor of the PDGF/VEGF family, and to be useful in situations where prevention or reduction of the PDGF-C growth factor or PDGF/VEGF family growth factor action is desirable.

Such situations include the tissue remodeling that takes place during invasion of tumor cells into a normal cell population by primary or metastatic tumor formation. Thus such the PDGF-C or PDGF/VEGF family growth factor-binding but non-mitogenic, non-differentiation inducing, non-migration inducing, non-motility inducing, non-survival promoting, non-connective tissue promoting, non-wound healing or non-vascular proliferation inducing variants of the PDGFC growth factor are also within the scope of the invention, and are referred to herein as “the PDGF-C growth factor-dimer forming but otherwise inactive or interfering variants”.

An example of a PDGF-C growth factor-dimer forming but otherwise inactive or interfering variant is where the PDGF-C has a mutation which prevents cleavage of CUB domain from the protein. It is further contemplated that a PDGF-C growth factor-dimer forming but otherwise inactive or interfering variant could be made to comprise a monomer, preferably an activated monomer, of VEGF, VEGF-B, VEGF-C, VEGF-D, PDGF-C, PDGF-A, PDGF-B or PlGF linked to a CUB domain that has a mutation which prevents cleavage of CUB domain from the protein. Dimers formed with the above mentioned PDGF-C growth factor-dimer forming but otherwise inactive or interfering variants and the monomers linked to the mutant CUB domain would be unable to bind to their corresponding receptors.

A variation on this contemplation would be to insert a proteolytic site between an activated monomer of VEGF, VEGF-B, VEGF-C, VEGF-D, PDGF-C, PDGF-A, PDGF-B or PlGF and the mutant CUB domain linkage which is dimerized to an activated monomer of VEGF, VEGF-B, VEGF-C, VEGF-D, PDGF-C, PDGF-A, PDGF-B or PlGF. An addition of the specific protease(s) for this proteolytic site would cleave the CUB domain and thereby release an activated dimer that can then bind to its corresponding receptor. In this way, a controlled release of an activated dimer is made possible.

The invention also relates to a purified and isolated nucleic acid encoding a polypeptide or polypeptide fragment of the invention as defined above. The nucleic acid may be DNA, genomic DNA, cDNA or RNA, and may be single-stranded or double stranded. The nucleic acid may be isolated from a cell or tissue source, or of recombinant or synthetic origin. Because of the degeneracy of the genetic code, the person skilled in the art will appreciate that many such coding sequences are possible, where each sequence encodes the amino acid sequence shown in FIG. 2 (SEQ ID NO:3), FIG. 4 (SEQ ID NO:5) or FIG. 6 (SEQ ID NO:7), a bioactive fragment or analog thereof, a receptor-binding but otherwise inactive or partially inactive variant thereof or a PDGF-C-dimer forming but otherwise inactive or interfering variants thereof.

Further, the invention provides vectors comprising the cDNA of the invention or a nucleic acid molecule according to the third aspect of the invention, and host cells transformed or transfected with nucleic acids molecules or vectors of the invention. These may be eukaryotic or prokaryotic in origin. These cells are particularly suitable for expression of the polypeptide of the invention, and include insect cells such as Sf9 cells, obtainable from the American Type Culture Collection (ATCC SRL-171), transformed with a baculovirus vector, and the human embryo kidney cell line 293-EBNA transfected by a suitable expression plasmid.

Preferred vectors of the invention are expression vectors in which a nucleic acid according to the invention is operatively connected to one or more appropriate promoters and/or other control sequences, such that appropriate host cells transformed or transfected with the vectors are capable of expressing the polypeptide of the invention. Other preferred vectors are those suitable for transfection of mammalian cells, or for gene therapy, such as adenoviral-, vaccinia- or retroviral-based vectors or liposomes. A variety of such vectors is known in the art.

The invention also relates to antibodies specifically reactive with a polypeptide of the invention or a fragment of the polypeptide. This aspect of the invention includes antibodies specific for the variant forms, immunoreactive fragments, analogs and recombinants of PDGF-C. Such antibodies are useful as inhibitors or agonists of PDGF-C and as diagnostic agents for detecting and quantifying PDGF-C. Polyclonal or monoclonal antibodies may be used.

Monoclonal and polyclonal antibodies can be raised against polypeptides of the invention or fragment or analog thereof using standard methods in the art. In addition the polypeptide can be linked to an epitope tag, such as the FLAG® octapeptide (Sigma, St. Louis, Mo.), to assist in affinity purification. For some purposes, for example where a monoclonal antibody is to be used to inhibit effects of PDGF-C in a clinical situation, it may be desirable to use humanized or chimeric monoclonal antibodies. Such antibodies may be further modified by addition of cytotoxic or cytostatic drugs. Methods for producing these, including recombinant DNA methods, are also well known in the art. This aspect of the invention also includes an antibody which recognizes PDGF-C and is suitably labeled.

Polypeptides or antibodies according to the invention may be labeled with a detectable label, and utilized for diagnostic purposes. Similarly, the thus-labeled polypeptide of the invention may be used to identify its corresponding receptor in situ. The polypeptide or antibody may be covalently or non-covalently coupled to a suitable supermagnetic, paramagnetic, electron dense, ecogenic or radioactive agent for imaging. For use in diagnostic assays, radioactive or non-radioactive labels may be used. Examples of radioactive labels include a radioactive atom or group, such as ¹²⁵I or ³²P. Examples of non-radioactive labels include enzymatic labels, such as horseradish peroxidase or fluorimetric labels, such as fluorescein-5-isothiocyanate (FITC). Labeling may be direct or indirect, covalent or non-covalent.

Clinical applications of the invention include diagnostic applications, acceleration of angiogenesis in tissue or organ transplantation, or stimulation of wound healing, or connective tissue development, or to establish collateral circulation in tissue infarction or arterial stenosis, such as coronary artery disease, and inhibition of angiogenesis in the treatment of cancer or of diabetic retinopathy and inhibition of tissue remodeling that takes place during invasion of tumor cells into a normal cell population by primary or metastatic tumor formation.

PDGF-C may also be relevant to a variety of lung conditions. PDGF-C assays could be used in the diagnosis of various lung disorders. PDGF-C could also be used in the treatment of lung disorders to improve blood circulation in the lung and/or gaseous exchange between the lungs and the blood stream. Similarly, PDGF-C could be used to improve blood circulation to the heart and O₂ gas permeability in cases of cardiac insufficiency. In a like manner, PDGF-C could be used to improve blood flow and gaseous exchange in chronic obstructive airway diseases.

The proliferation of vascular endothelial cells, formation and spreading of blood vessels, or vasculogenesis and angiogenesis, respectively, play important roles in a variety of physiological processes such as embryonic development, wound healing, organ regeneration and female reproductive processes such as follicle development in the corpus luteum during ovulation and placental growth after pregnancy. Uncontrolled angiogenesis can be pathological such as in the growth of solid tumors that rely on vascularization for growth.

As discussed above, millions of patients per year in the U.S. suffer from myocardial infarction (MI) and/or critical limb ischemia. Many millions more suffer from related syndromes due to atherosclerosis. Many of these patients will benefit from the ability to stimulate collateral vessel formation in ischemic areas.

In one embodiment of the invention, a polypeptide having a PDGF-C core domain activity (a truncated active form) is administered in vivo to stimulate or enhance vasculogenesis, arteriogenesis and angiogenesis, respectively. Furthermore, administration of the PDGF-C core domain or a fragment having an activity thereof promotes angiogenesis and/or arteriogenesis and/or vasculogenesis, and may further be used to promote wound healing.

Where a composition is to be used for therapeutic purposes, the dose(s) and route of administration will depend upon the nature of the patient and condition to be treated, and will be at the discretion of the attending physician or veterinarian. Suitable routes include oral, subcutaneous, intramuscular, intraperitoneal or intravenous injection, parenteral, topical application, implants etc. Topical application may be used. For example, where used for wound healing or other use in which enhanced angiogenesis is advantageous, an effective amount of the truncated active form of PDGF-C is administered to an organism in need thereof in a dose between about 0.1 and 1000 mg/kg body weight.

The compounds may be employed in combination with a suitable pharmaceutical carrier. The resulting compositions comprise a therapeutically effective amount of a compound, and a pharmaceutically acceptable solid or liquid carrier or adjuvant. Examples of such a carrier or adjuvant include, but are not limited to, saline, buffered saline, Ringer's solution, mineral oil, talc, corn starch, gelatin, lactose, sucrose, microcrystalline cellulose, kaolin, mannitol, dicalcium phosphate, sodium chloride, alginic acid, dextrose, water, glycerol, ethanol, thickeners, stabilizers, suspending agents and combinations thereof.

Such compositions may be in the form of solutions, suspensions, tablets, capsules, creams, salves, elixirs, syrups, wafers, ointments or other conventional forms. The formulation to suit the mode of administration. Compositions which comprise PDGF-C may optionally further comprise one or more of PDGF-A, PDGF-B, VEGF, VEGF-B, VEGF-C, VEGF-D, PlGF and/or heparin. Compositions comprising PDGF-C will contain from about 0.1% to 90% by weight of the active compound(s), and most generally from about 10% to 30%.

For intramuscular preparations, a sterile formulation can be dissolved and administered in a pharmaceutical diluent such as pyrogen-free water (distilled), physiological saline or 5% glucose solution. A suitable insoluble form of the compound may be prepared and administered as a suspension in an aqueous base or a pharmaceutically acceptable oil base, e.g. an ester of a long chain fatty acid such as ethyl oleate.

Another aspect of the invention relates to the discovery that the full length PDGF-C protein is a latent growth factor that needs to be activated by proteolytic processing to release an active PDGF/VEGF homology domain. A putative proteolytic site is found in residues 231-234 in the full length protein, residues -RKSR-. This is a dibasic motif. This site is structurally conserved in the mouse PDGF-C. The -RKSR- putative proteolytic site is also found in PDGF-A, PDGF-B, VEGF-C and VEGF-D. In these four proteins, the putative proteolytic site is also found just before the minimal domain for the PDGF/VEGF homology domain. Together these facts indicate that this is the proteolytic site.

Preferred proteases include, but are not limited, to plasmin, Factor X and enterokinase. The N-terminal CUB domain may function as an inhibitory domain which might be used to keep PDGF-C in a latent form in some extracellular compartment and which is removed by limited proteolysis when PDGF-C is needed.

Polynucleotides of the invention such as those described above, fragments of those polynucleotides, and variants of those polynucleotides with sufficient similarity to the non-coding strand of those polynucleotides to hybridize thereto under stringent conditions all are useful for identifying, purifying, and isolating polynucleotides encoding other, non-human, mammalian forms of PDGF-C. Thus, such polynucleotide fragments and variants are intended as aspects of the invention. Exemplary stringent hybridization conditions are as follows: hybridization at 42° C. in 5×SSC, 20 mM NaPO₄, pH 6.8, 50% formamide; and washing at 42° C. in 0.2×SSC. Those skilled in the art understand that it is desirable to vary these conditions empirically based on the length and the GC nucleotide base content of the sequences to be hybridized, and that formulas for determining such variation exist. See for example Sambrook et al, “Molecular Cloning: A Laboratory Manual,” Second Edition, pages 9.47-9.51, Cold Spring Harbor, N.Y.: Cold Spring Harbor Laboratory (1989).

Moreover, purified and isolated polynucleotides encoding other, non-human, mammalian PDGF-C forms also are aspects of the invention, as are the polypeptides encoded thereby and antibodies that are specifically immunoreactive with the non-human PDGF-C variants. Thus, the invention includes a purified and isolated mammalian PDGF-C polypeptide and also a purified and isolated polynucleotide encoding such a polypeptide.

It will be clearly understood that nucleic acids and polypeptides of the invention may be prepared by synthetic means or by recombinant means, or may be purified from natural sources.

It will be clearly understood that for the purposes of this specification the word “comprising” means “including but not limited to.” The corresponding meaning applies to the word “comprises.”

### BRIEF DESCRIPTION OF THE DRAWINGS

FIG. 1 (SEQ ID NO:2) shows the complete nucleotide sequence of cDNA encoding a human PDGF-C (hPDGF-C)(2108 bp).

FIG. 2 (SEQ ID NO:3) shows the deduced amino acid sequence of full-length hPDGF-C which consists of 345 amino acid residues (the translated part of the cDNA corresponds to nucleotides 37 to 1071 of FIG. 1).

FIG. 3 (SEQ ID NO:4) shows a cDNA sequence encoding a fragment of human PDGF-C (hPDGF-C)(1536 bp).

FIG. 4 (SEQ ID NO:5) shows a deduced amino acid sequence of a fragment of hPDGF-C (translation of nucleotides 3 to 956 of the nucleotide sequence of FIG. 3).

FIG. 5 (SEQ ID NO:6) shows a nucleotide sequence of a murine PDGF-C (mPDGF-C) cDNA.

FIG. 6 (SEQ ID NO:7) shows the deduced amino acid sequence of a fragment of mPDGF-C (the translated part of the cDNA corresponds to nucleotides 196 to 1233 of FIG. 5).

FIG. 7 shows a comparative sequence alignment of the hPDGF-C amino acid sequence of FIG. 2 (SEQ ID NO:3) with the mPDGF-C amino acid sequence of FIG. 6 (SEQ ID NO:7).

FIG. 8 shows a schematic structure of mPDGF-C with a signal sequence (striped box), a N-terminal C1r/C1s/embryonic sea urchin protein Uegf/bone morphogenetic protein 1 (CUB) domain and the C-terminal PDGF/VEGF-homology domain (open boxes).

FIG. 9 shows a comparative sequence alignment of the PDGF/VEGF-homology domains in human and mouse PDGF-C with other members of the VEGF/PDGF family of growth factors (SEQ ID NOs:8-17, respectively).

FIG. 10 shows a phylogenetic tree of several growth factors belonging to the VEGF/PDGF family.

FIG. 11 provides the amino acid sequence alignment of the CUB domain present in human and mouse PDGF-Cs (SEQ ID NOs:18 and 19, respectively) and other CUB domains present in human bone morphogenic protein-1 (hBMP-1, 3 CUB domains CUB1-3)(SEQ ID NOs:20-22, respectively) and in human neuropilin-1 (2 CUB domains)(SEQ ID NOs:23 and 24, respectively).

FIG. 12 shows a Northern blot analysis of the expression of PDGF-C transcripts in several human tissues.

FIG. 13 shows the regulation of PDGF-C mRNA expression by hypoxia.

FIG. 14 shows the expression of PDGF-C in human tumor cell lines.

FIG. 15 shows the results of immunoblot detection of full length human PDGF-C in transfected COS-1 cells.

FIG. 16 shows isolation and partial characterization of full length PDGF-C.

FIG. 17 shows isolation and partial characterization of a truncated form of human PDGF-C containing the PDGF/VEGF homology domain only.

FIG. 18 provides a standard curve for the binding of labeled PDGF-BB homodimers to PAE-1 cells expressing PDGF alpha receptor.

FIG. 19 provides a graphic representation of the inhibition of binding of labeled PDGF-BB to PAE-1 cells expressing PDGF alpha receptor by increasing amounts of purified full length and truncated PDGF-CC proteins.

FIG. 20 shows the effects of the full length and truncated PDGF-CC homodimers on the phosphorylation of PDGF alpha-receptor.

FIG. 21 shows the mitogenic activities of the full length and truncated PDGF-CC homodimers on fibroblasts.

FIG. 22 graphically presents the results of the binding assay of truncated PDGF-C to the PDGF receptors.

FIG. 23 shows the immunoblot of the undigested full length PDGF-C protein and the plasmin-generated 26-28 kDa species.

FIG. 24 graphically presents the results of the competitive binding assay of full-length PDGF-C and truncated PDGF-C for PDGFR-alpha receptors.

FIG. 25 shows the analyses by SDS-PAGE of the human PDGF-C CUB domain under reducing and non-reducing conditions.

FIGS. 26A-26V show PDGF-C expression in the developing mouse embryo.

FIGS. 27A-27F show PDGF-C, PDGF-A and PDGFR-alpha expression in the developing kidney.

FIGS. 28A-28F show histology of E 16.5 kidneys from wildtype (FIGS. 28A and 28C), PDGFR-alpha −/− (FIGS. 28B and 28F, PDGF-A −/− (FIG. 28D) and PDGF-A/PDGF-B double −/− (FIG. 28E) kidneys.

FIG. 29 shows an immunoblot analysis of conditioned medium from 1523 fibroblasts. Note the two principal Mr 25 kDa species and the weak band of Mr 55 kDa corresponding to full length PDGF-C.

FIG. 30 shows an immunoblot analysis of recombinant full length PDGF-C and conditioned medium from 1523 fibroblasts using an antibody to the His₆ epitope. Note the low, but significant, endogenous processing of full length PDGF-C, and the absence of His₆ epitopes in proteins in the medium from 1523 cells.

FIG. 31 shows results of protease inhibitor profiling for processing of full length PDGF-C. The data show that the conditioned medium from 1523 fibroblasts contains a serine protease with trypsin-like properties that is responsible for processing of PDGF-C.

FIGS. 32A and B show smooth muscle cell alpha actin staining in normal (32A) and PDGF-C treated (32B) hearts after infarction.

FIG. 33 shows Vessel densities in the infarcted heart area in untreated (N, While) and PDGF-C treated (P, solid black) mice.

FIG. 34 shows capillary density in the infarcted area 7 days following the induction of myocardial infarction in mice, treated (black bars) or un-treated (white bars) with 30 μg of recombinant PDGF-C delivered via a mini-osmotic pump.

FIG. 35 shows the density of smooth muscle α-actin coated vessels in the infarcted area 7 days following the induction of myocardial infarction in mice, treated (black bars) or un-treated (white bars) with 30 μg of recombinant PDGF-C delivered via a mini-osmotic pump.

FIG. 36 shows therapeutic angiogenesis and arteriogenesis with PDGF-CC in ischemic heart. a, RNAse protection analysis (RPA) showed that PDGFR-α transcripts were detectable in the normal mouse heart. β-actin was used as an internal control. b, Upper and middle panels: immunoprecipitation and subsequent Western blotting for PDGFR-α (upper) and phospho-tyrosine (pTyr; middle) showed that PDGFR-α was upregulated in the ischemic myocardial regions, bordering the infarct where vessels start to grow. Note also that PDGFR-α was activated more in the border zones than in the normal (non-ischemic) regions of the heart, and maximally after PDGF-CC treatment. Lower panel: Coomassie staining revealed comparable loading. c,d, PDGF-CC protein treatment increased vascular density in the infarcted areas in a dosage dependent way. TM-positive vessel density was significantly increased after 4.5 μg/day PDGF-CC treatment, while the effect of 1.5 μg/day PDGF-CC was minimal (c). SMA-positive vessel density was increased by 1.5 μg/day PDGF-CC treatment, and the increase was greater when 4.5 μg/day PDGF-CC was used (d). *: P<0.05. Values are presented as mean±SEM of at least 7 mice. e-g, Thrombomodulin (TM) was used as a marker to quantify the total number of vessels (brown color). Compared with vehicle (e) and 1.5 μg/day (f), 4.5 μg/day PDGF-CC treatment significantly increased vessel density (g). h-j, Smooth muscle cell alpha-actin (SMA) was used as a marker to quantify the number of arterioles in the infarcted area. Compared with vehicle (h), 1.5 μg/day PDGF-CC treatment significantly increased vessel density (i), with a greater effect after 4.5 μg/day PDGF-CC treatment (j). No signs of edema, hemorrhage or fibrosis were observed. Scale bars: 50 μm

FIG. 37 shows the therapeutic angiogenesis with PDGF-CC in limb ischemia. a, Quantitative RNAse protection analysis using β-actin as an internal control showed that PDGFR-α expression in the gastrocnemius muscle was decreased at two days after femoral artery ligation, but almost restored to normal level by 4.5 μg/day PDGF-CC treatment. The ratio of the PDGFR-α levels (arbitrary units), normalized for β-actin levels, is shown. b,c, PDGF-CC protein treatment increased the PECAM⁺ capillary (b) and SMA⁺ arteriolar (c) density in the ischemic gastrocnemius muscles. d,e, PDGF-CC protein treatment decreased muscle necrosis (d) and increased muscle regeneration (e) in the gastrocnemius muscle at seven days after femoral artery ligation. Necrotic muscle fibers were identified as ghost cells lacking nuclei and containing a hyaline cytosol; regenerating myocytes were identified as small cells with central nuclei. Areas are expressed as percentage of the total muscle area. f,g, Compared with vehicle (f), PDGF-CC protein treatment significantly increased the density of CD31/PECAM-positive vessels in the regenerating areas of the ischemic gastrocnemius muscle (g). No signs of edema, hemorrhage or fibrosis were observed. h,i, H&E staining, displaying large areas of regenerating myocytes (small cells with central nuclei) after PDGF-CC treatment (i) than vehicle (h). The regions containing regenerating myocytes are surrounded by a dashed yellow line in both panels. j-l, Higher magnification of H&E stained-muscle sections of a normal muscle (j) and after femoral artery ligation (k,l): (j) normal gastrocnemius muscle, containing well organized myocytes and peripheral nuclei (arrowheads); (k) ischemic muscle, treated with vehicle, containing numerous necrotic ghost-myocytes with a hyaline cytosol without identifiable nucleus (arrowheads) and few blood vessels; (l) ischemic muscle, treated with PDGF-CC, containing numerous regenerating myocytes with central nuclei (arrowheads) and numerous blood vessels. Panels a-e: *: P<0.05. Values are presented as mean±SEM of at least 15 mice. The lumen of the arterioles is filled with dark bismuth gelatin in panels f-l. Scale bars: 50 μm.

FIG. 38 shows that PDGF-CC mobilizes endothelial progenitors (EPCs) in tissue ischemia in vivo. a, PDGF-CC treatment significantly augmented EPC mobilization by ˜4-fold, as compared with the vehicle group, from day 2 to day 5 after hind limb ischemia, but did not affect EPC mobilization in normal conditions. *: P<0.05. Values are presented as mean±SEM of 10 mice. b-g: Isolectin-IB4 (green) and AcLDL-DiI (red) double staining was used to count the number of EPCs. Note the sparse positive cells in the vehicle group (b-d), and the higher density of the AcLDL-DiI/Isolectin-IB4 double positive cells (yellow) after PDGF-CC treatment (e-g).

FIG. 39 shows that PDGF-CC promotes differentiation of adult bone marrow cells into EC and SMC cells. a, Human adult bone marrow-derived AC133⁺CD34⁺ cells were stimulated with PDGF-CC or VEGF. After two weeks, both PDGF-CC and VEGF enhanced the adherence of the cells, measured by a luminescence assay (see methods). *: P<0.05. Values are presented as mean±SEM. b-m, After two weeks of stimulation, both PDGF-CC (g,j) and VEGF (f,i) induced the expression of EC surface markers CD144 (VE-cadherin) and CD31 (PECAM), while vehicle treated cells remained negative (e,h). Only PDGF-CC induced prominent SMA expression (m), while cells treated with VEGF (l) or vehicle (k) displayed background level of SMA expression. Panels b-d show unstained cells.

FIG. 40 shows that PDGF-CC promotes EC migration but not proliferation. a,b, In both cultured HMVECs (a) and BAECs (b), PDGF-CC promoted EC migration with a similar potency as VEGF, while PDGF-AA had no effect. c, PDGF-CC, like PDGF-AA, had no effect on the proliferation of the ECs, while VEGF potently promoted cell proliferation. *: P<0.05. Values are presented as mean±SEM.

FIG. 41 shows that PDGF-CC stimulates outgrowth of microvessels and perivascular cells in the aortic ring assay. a-e, Micrographs of aortic rings, displaying microvascular sprouts and perivascular cells. Compared to vehicle (a), VEGF stimulated microvascular outgrowth (b), while PDGF-CC enhanced the outgrowth of both microvascular sprouts and fibroblast-like cells (c-e). At 5-10 ng/ml, PDGF-CC maximally stimulated perivascular fibroblast-like cells, which emigrated over much greater distances from the aortic ring (c,d). At high concentrations (30-50 ng/ml), PDGF-CC still stimulated fibroblast-like cell growth and emigration but less significantly than at lower concentrations, possibly because the perivascular cells were recruited by the sprouting microvessels (e). f-k, Quantification of the outgrowth of microvascular sprouts (f-h) and perivascular fibroblast-like cells (i-k), using computer-assisted morphometry. Panels f-h: VEGF increased both the number of sprouting microvessels and the distance over which they grew out (f); PDGF-CC increased the number of microvessels at 30 ng/ml (g), while PDGF-AA had no effect on the number of microvessels (h). Panels i-k: Unlike VEGF, which was ineffective on perivascular fibroblast-like cells (i), PDGF-CC increased the number and migration of the perivascular cells over much greater distances from the aortic ring (j), while PDGF-AA has an intermediate effect (k).

FIG. 42 shows that PDGF-CC is a potent mitogen for cultured SMCs and fibroblasts and upregulates VEGF expression. a, Immunoprecipitation with anti-PDGFR-α followed by Western blotting for PDGFR-α (upper panel) or phospho-tyrosine residues (pTyr, lower panel) revealed that cultured hSMC and NIH-3T3 fibroblast cells expressed significant amount of tyrosine-phosphorylated (active) PDGFR-α. b, Both PDGF-CC and AA stimulated hSMC migration with a similar potency, while VEGF had no effect. c,d, PDGF-CC and -AA stimulated the proliferation of cultured NIH-3T3 fibroblast cells (c) and hSMCs (d). e, PDGF-C was overexpressed in the NIH-3T3 fibroblast cells as confirmed by Western blotting (lower panel). VEGF mRNA level was significantly upregulated in the PDGF-CC over-expressing cells as compared to that of vector transduced cells, using β-actin as an internal control (upper panels). If ELISA immunoassay confirmed that secreted VEGF protein level in the serum-free PDGF-CC over-expressing cell conditioned media was significantly increased as compared with that of the vector transduced cell conditioned media. *: P<0.05. Values are presented as mean±SEM.

### DETAILED DESCRIPTION OF PREFERRED EMBODIMENTS

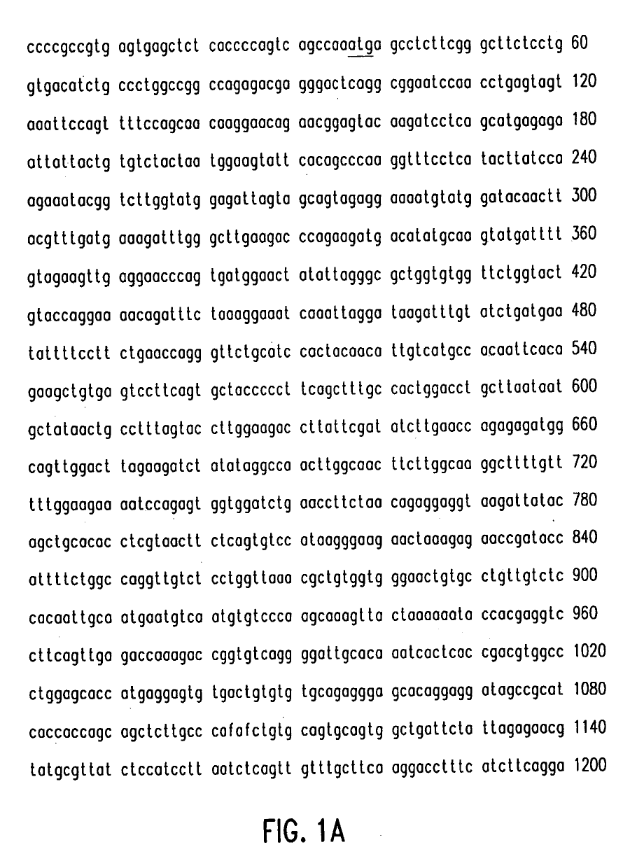

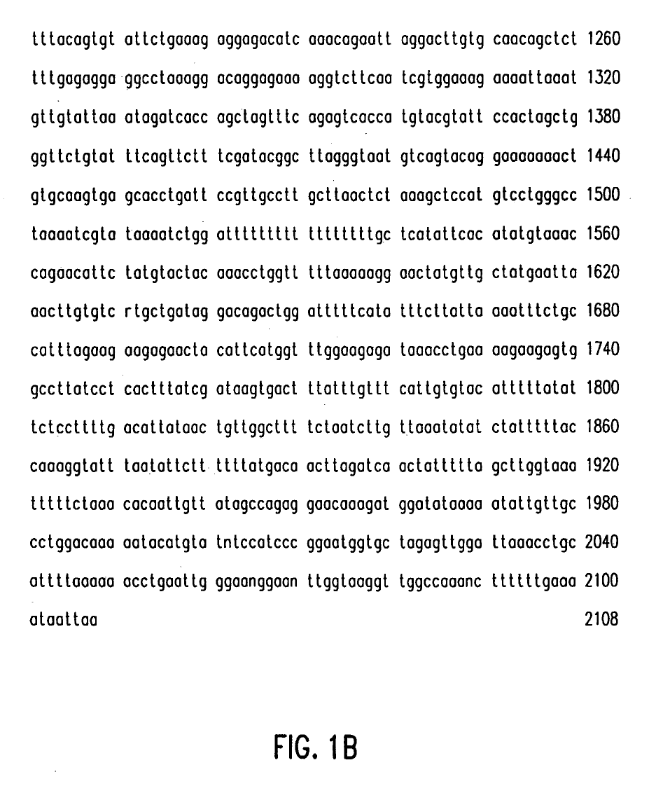

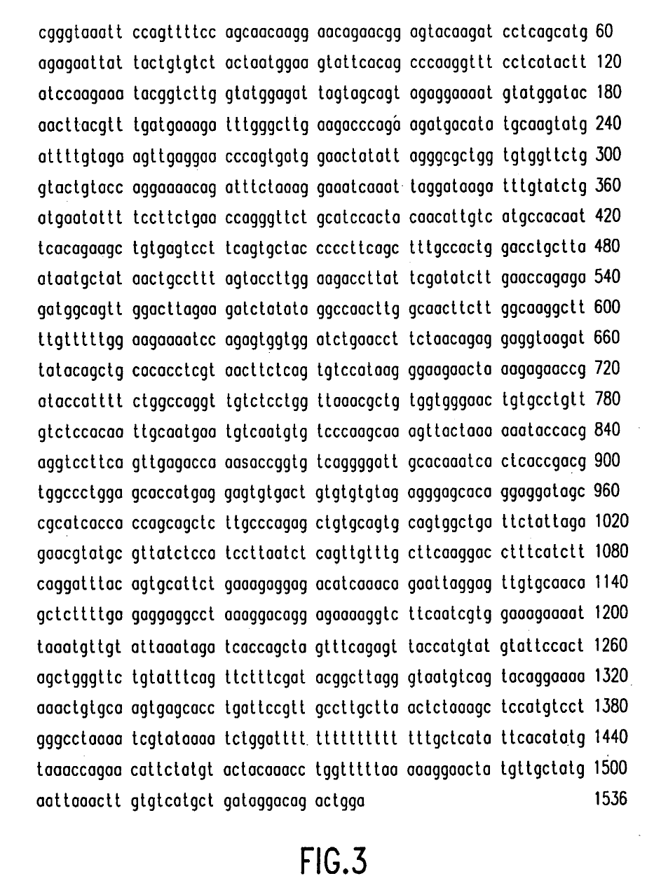

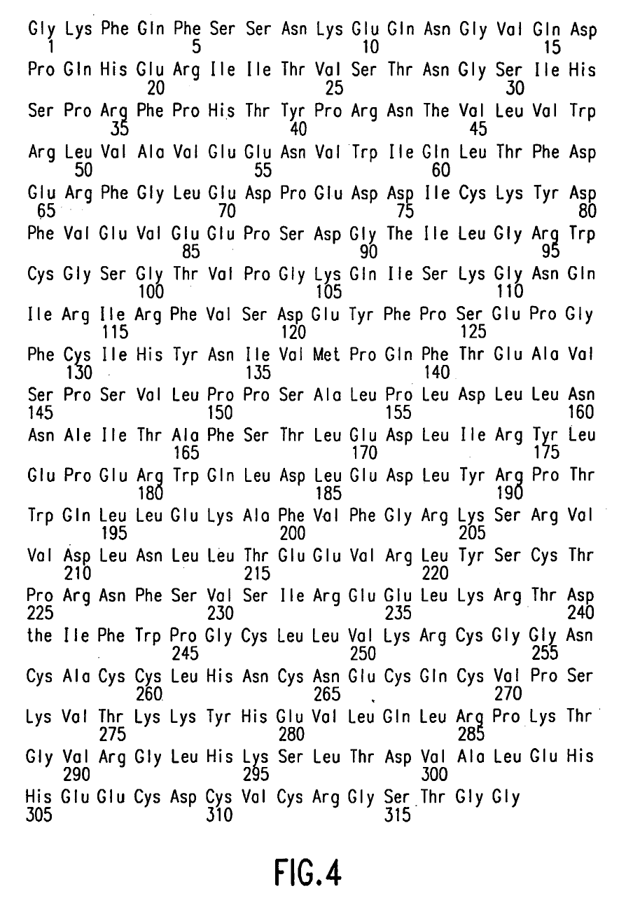

FIG. 1 (SEQ ID NO:2) shows the complete nucleotide sequence of cDNA encoding a human PDGF-C (hPDGF-C)(2108 bp), which is a new member of the VEGF/PDGF family. A clone #4 (see FIGS. 3 and 4—SEQ ID NOs:4 and 5) encoding hPDGF-C was not full length and lacked approximately 80 base pairs of coding sequence when compared to the mouse protein (corresponding to 27 amino acids). Additional cDNA clones were isolated from a human fetal lung cDNA library to obtain an insert which included this missing sequence. Clone #10 had a longer insert than clone #4. The insert of clone #10 was sequenced in the 5′ region and it was found to contain the missing sequence. Clone #10 was found to include the full sequence of human PDGF-C. Some 5′-untranslated sequence, the translated part of the cDNA encoding human PDGF-C and some 3′-untranslated nucleotide sequence are shown in FIG. 1 (SEQ ID NO:2). A stop codon in frame is located 21 bp upstream of the initiation ATG (the initiation ATG is underlined in FIG. 1).

Work to isolate this new human PDGF/VEGF began after a search of the expressed sequence tag (EST) database, dbEST, at the National Center for Biotechnology Information (NCBI) in Washington, D.C., identified a human EST sequence (W21436) which appears to encode part of the human homolog of the mouse PDGF-C. Based on the human EST sequence, two oligonucleotides were designed:

| | |
|---|---|
| 5′-GAA GTT GAG GAA CCC AGT G-3′ | (SEQ ID NO:25) |
| forward | |
| 5′-CTT GCC AAG AAG TTG CCA AG-3′ | (SEQ ID NO:26) |
| reverse. | |

These oligonucleotides were used to amplify by polymerase chain reaction (PCR) a polynucleotide of 348 bps from a Human Fetal Lung 5′-STRETCH PLUS λgt10 cDNA library, which was obtained commercially from Clontech. The PCR product was cloned into the pCR 2.1-vector of the Original TA Cloning Kit (Invitrogen). Subsequently, the 348 bps cloned PCR product was used to construct a hPDGF-C probe according to standard techniques.

10⁶ lambda-clones of the Human Fetal Lung 5′-STRETCH PLUS λgt10 cDNA Library (Clontech) were screened with the hPDGF-C probe according to standard procedures. Among several positive clones, one, clone #4 was analyzed more carefully and the nucleotide sequence of its insert was determined according to standard procedures using internal and vector oligonucleotides. The insert of clone #4 contains a partial nucleotide sequence of the cDNA encoding the full length human PDGF-C (hPDGF-C). The nucleotide sequence (1536 bp) of the clone #4 insert is shown in FIG. 3 (SEQ ID NO:4). The translated portion of this cDNA includes nucleotides 6 to 956. The deduced amino acid sequence of the translated portion of the insert is illustrated in FIG. 4 (SEQ ID NO:5). A polypeptide of this deduced amino acid sequence would lack the first 28 amino acid residues found in the full length hPDGF-C polypeptide. However, this polypeptide includes a proteolytic fragment which is sufficient to activate the PDGF alpha receptors. It should be noted that the first glycine (Gly) of SEQ ID NO:5 is not found in the full length hPDGF-C.

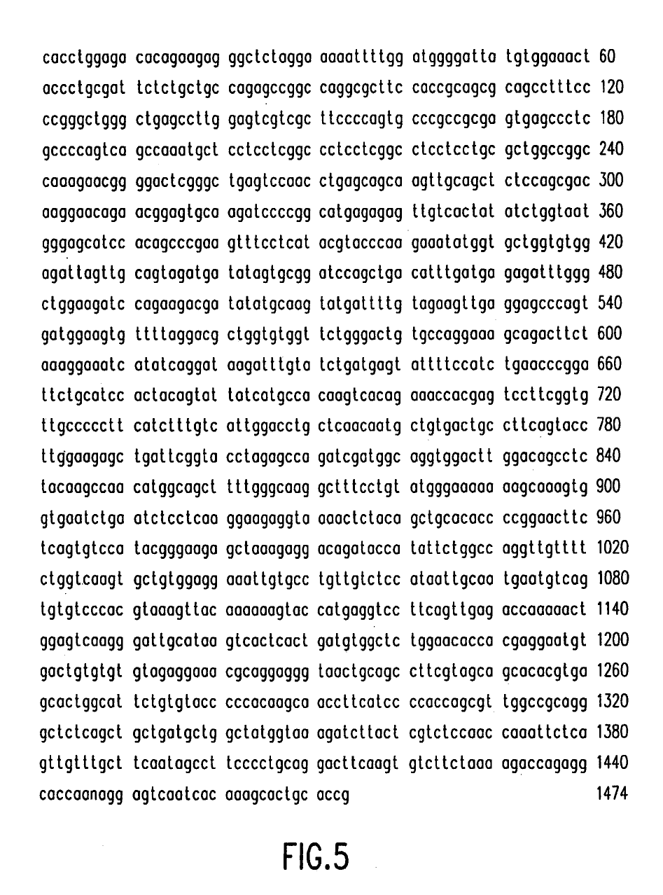

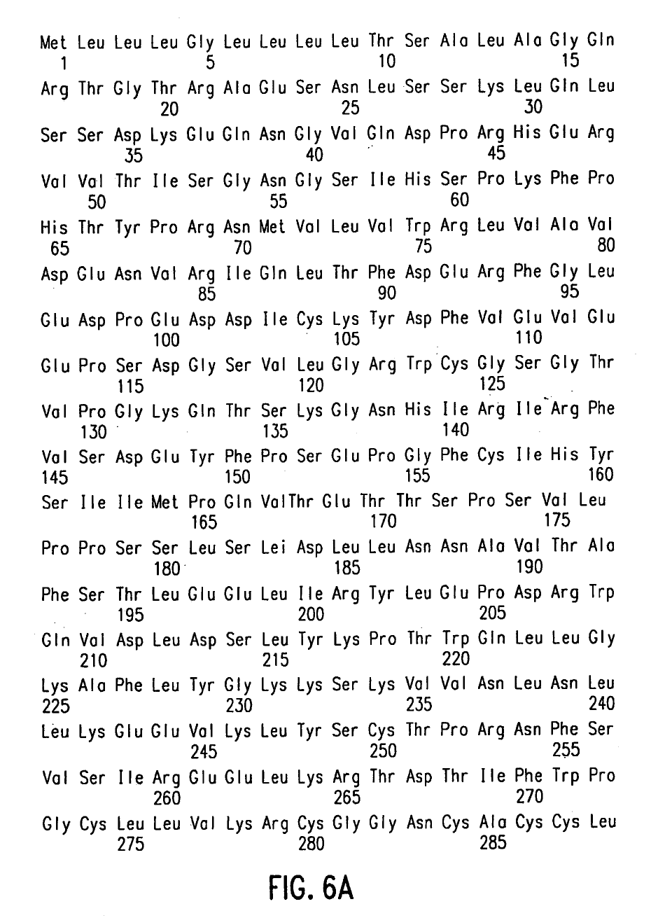

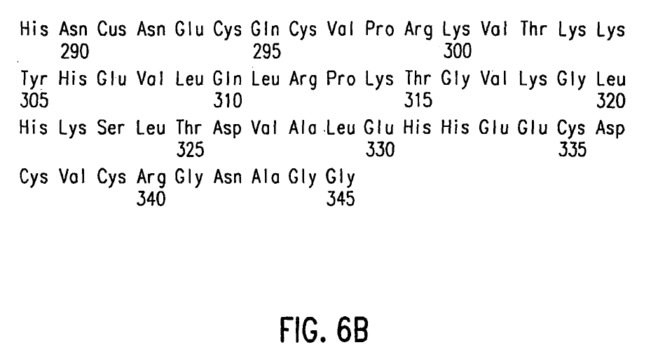

A mouse EST sequence (AI020581) was identified in a database search of the dbEST database at the NCBI in Washington, D.C., which appears to encode part of a new mouse PDGF, PDGF-C. Large parts of the mouse cDNA was obtained by PCR amplification using DNA from a mouse embryo λgt10 cDNA library as the template. To amplify the 3′ end of the cDNA, a sense primer derived from the mouse EST sequence was used (the sequence of this primer was 5′-CTT CAG TAC CTT GGA AGA G, primer 1 (SEQ ID NO:27)) To amplify the 5′end of the cDNA, an antisense primer derived from the mouse EST was used (the sequence of this primer was 5′-CGC TTG ACC AGG AGA CAA C, primer 2 (SEQ ID NO:28)). The λgt10 vector primers were sense 5′-ACG TGA ATT CAG CAA GTT CAG CCT GGT TAA (primer 3 (SEQ ID NO:29)) and antisense 5′-ACG TGG ATC CTG AGT ATT TCT TCC AGG GTA (primer 4 (SEQ ID NO:30)). Combinations of the vector primers and the internal primers obtained from the mouse EST were used in standard PCR reactions. The sizes of the amplified fragments were approx. 750 bp (3′-fragment) and 800 bp (5′-fragment), respectively. These fragments were cloned into the pCR 2.1 vector and subjected to nucleotide sequences analysis using vector primers and internal primers. Since these fragments did not contain the full length sequence of mPDGF-C, a mouse liver ZAP cDNA library was screened using standard conditions. A 261 bp ³²P-labeled PCR fragment was generated for use as a probe using primers 1 and 2 and using DNA from the mouse embryo λgt10 library as the template (see above). Several positive plaques were purified and the nucleotide sequence of the inserts were obtained following subcloning into pBluescript. Vector specific primers and internal primers were used. By combining the nucleotide sequence information of the generated PCR clones and the isolated clone, the full length amino acid sequence of mPDGF-C could be deduced (see FIG. 6)(SEQ ID NO:7).

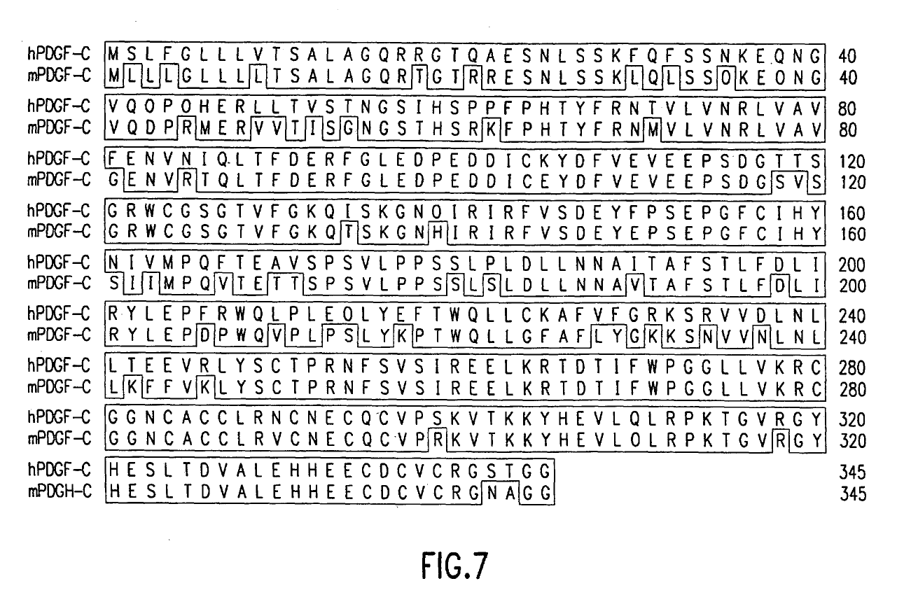

FIG. 7 shows a comparative sequence alignment of the mouse and human amino acid sequences of PDGF-C (SEQ ID NOS:6 and 2, respectively). The alignment shows that human and mouse PDGF-Cs display an identity of about 87% with 45 amino acid replacements found among the 345 residues of the full length proteins. Almost all of the observed amino acid replacements are conservative in nature. The predicted cleavage site in mPDGF-C for the signal peptidase is between residues G19 and T20. This would generate a secreted mouse peptide of 326 amino acid residues.

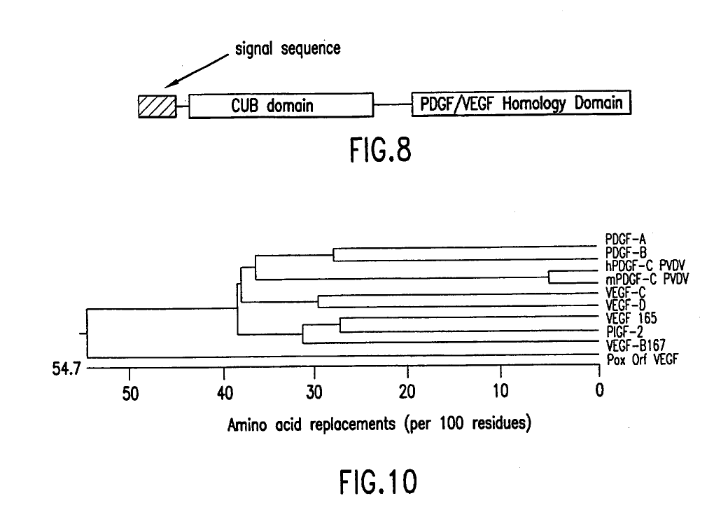

FIG. 8 provides a schematic domain structure of mouse PDGF-C with a signal sequence (striped box), a N-terminal CUB domain and the C-terminal PDGF/VEGF-homology domain (open boxes). The amino acid sequences denoted by the lines have no obvious similarities to CUB domains or to VEGF-homology domains.

The high sequence identity suggests that human and mouse PDGF-C have an almost identical domain structure. Amino acid sequence comparisons revealed that both mouse and human PDGF-C display a novel domain structure. Apart from the PDGF/VEGF-homology domain located in the C-terminal region in both proteins (residues 164 to 345), the N-terminal region in both PDGF-Cs have a domain referred to as a CUB domain (Bork and Beckmann, J. Mol. Biol., 1993 231:539-545). This domain of about 110 amino acids (amino acid residues 50-160) was originally identified in complement factors C1r/C1s, but has recently been identified in several other extracellular proteins including signaling molecules such as bone morphogenic protein 1 (BMP-1) (Wozney et al., Science, 1988 242:1528-1534) as well as in several receptor molecules such as neuropilin-1 (NP-1) (Soker et al., Cell, 1998 92:735-745). The functional roles of CUB domains are not clear but it may participate in protein-protein interactions or in interactions with carbohydrates including heparin sulfate proteoglycans.

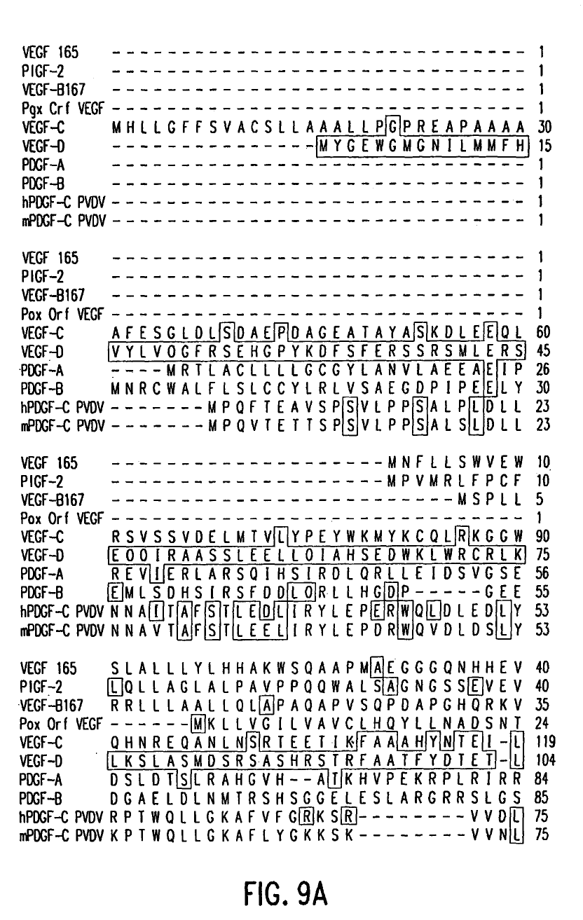

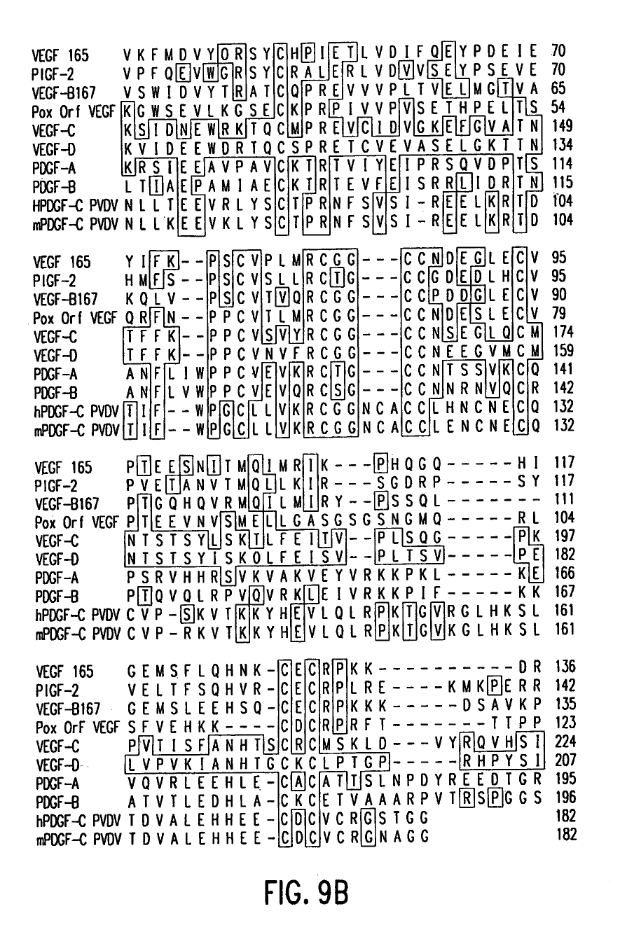

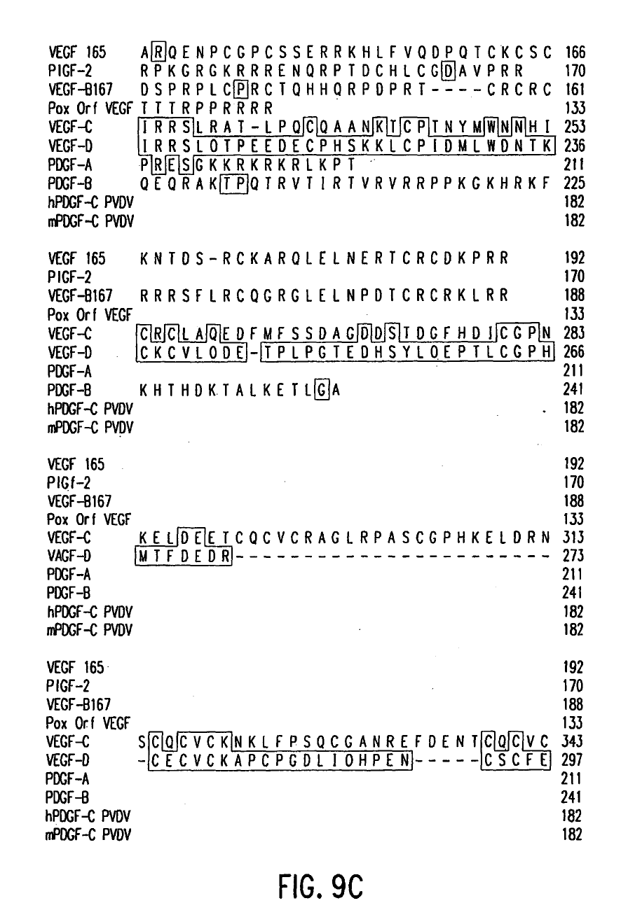

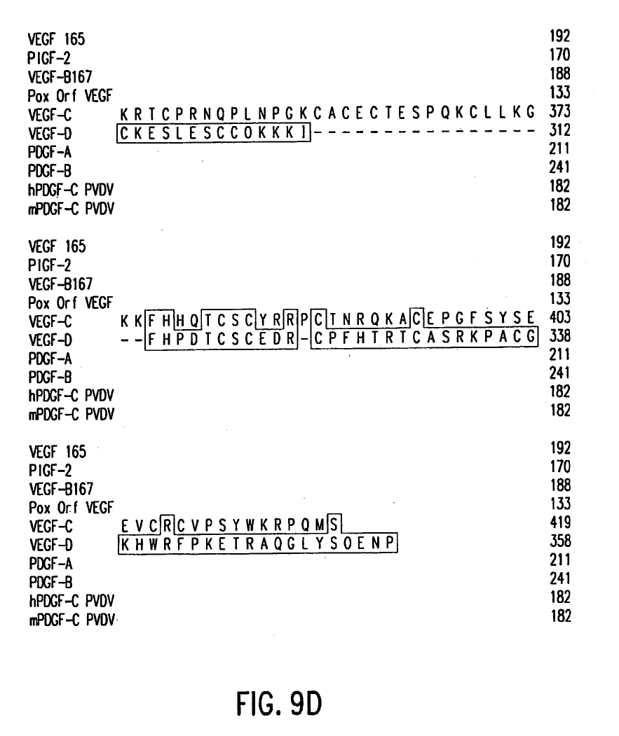

FIG. 9 shows the amino acid sequence alignment of the C-terminal PDGF/VEGF-homology domains of human and mouse PDGF-Cs with the C-terminal PDGF/VEGF-homology domains of PDGF/VEGF family members, VEGF₁₆₅, PlGF-2, VEGF-B₁₆₇, Pox Orf VEGF, VEGF-C, VEGF-D, PDGF-A and PDGF-B (SEQ ID NOs:8-17). Some of the amino acid sequences in the N- and C-terminal regions in VEGF-C and VEGF-D have been deleted in this figure. Gaps were introduced to optimize the alignment. This alignment was generated using the method of J. Hein, (Methods Enzymol. 1990 183:626-45) with PAM250 residue weight table. The boxed residues indicate amino acids which match the PDGF-Cs within two distance units.

The alignment shows that PDGF-C has the expected pattern of invariant cysteine residues, a hallmark of members of this family, with one exception. Between cysteine 3 and 4, normally spaced by 2 residues there is an insertion of three extra amino acids (NCA). This feature of the sequence in PDGF-C was highly unexpected.

Based on the amino acid sequence alignments in FIG. 9, a phylogenetic tree was constructed and is shown in FIG. 10. The data show that the PDGF-C homology domain is closely related to the PDGF/VEGF-homology domains of VEGF-C and VEGF-D.

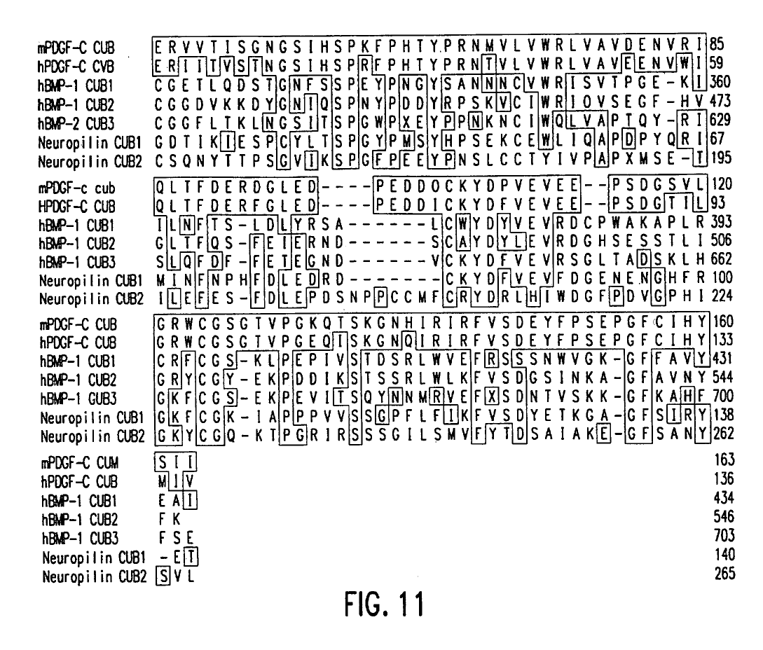

As shown in FIG. 11, the amino acid sequences from several CUB-containing proteins were aligned (SEQ ID NOs:18-24). The results show that the single CUB domain in human and mouse PDGF-C (SEQ ID NOs:18 and 19, respectively) displays a significant identify with the most closely related CUB domains. Sequences from human BMP-1, with 3 CUB domains (CUB1-3 (SEQ ID NOs:20-22)) and human neuropilin-1 with 2 CUB domains (CUB1-2)(SEQ ID NOs:23 and 24, respectively) are shown. Gaps were introduced to optimize the alignment. This alignment was generated using the method of J. Hein, (Methods Enzymol., 1990 183 626-45) with PAM250 residue weight table.

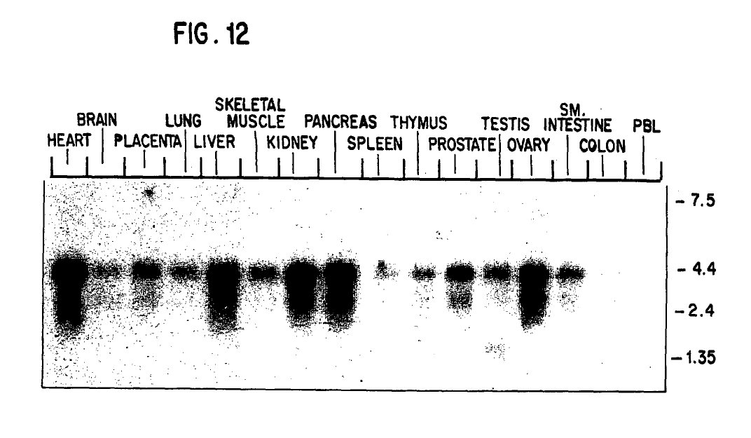

FIG. 12 shows a Northern blot analysis of the expression of PDGF-C transcripts in several human tissues. The analysis shows that PDGF-C is encoded by a major transcript of approximately 3.8-3.9 kb, and a minor of 2.8 kb. The numbers to the right refer to the size of the mRNAs (in kb). The tissue expression of PDGF-C was determined by Northern blotting using a commercial Multiple Tissue Northern blot (MTN, Clontech). The blots were hybridized at according to the instructions from the supplier using ExpressHyb solution at 68° C. for one hour (high stringency conditions), and probed with a 353 bp hPDGF-C EST probe from the fetal lung cDNA library screening as described above. The blots were subsequently washed at 50° C. in 2×SSC with 0.05% SDS for 30 minutes and at 50° C. in 0.1×SSC with 0.1% SDS for an additional 40 minutes. The blots were then put on film and exposed at −70° C. The blots show that PDGF-C transcripts are most abundant in heart, liver, kidney, pancreas and ovary while lower levels of transcripts are present in most other tissues, including placenta, skeletal muscle and prostate. PDGF-C transcripts were below the level of detection in spleen, colon and peripheral blood leucocytes.

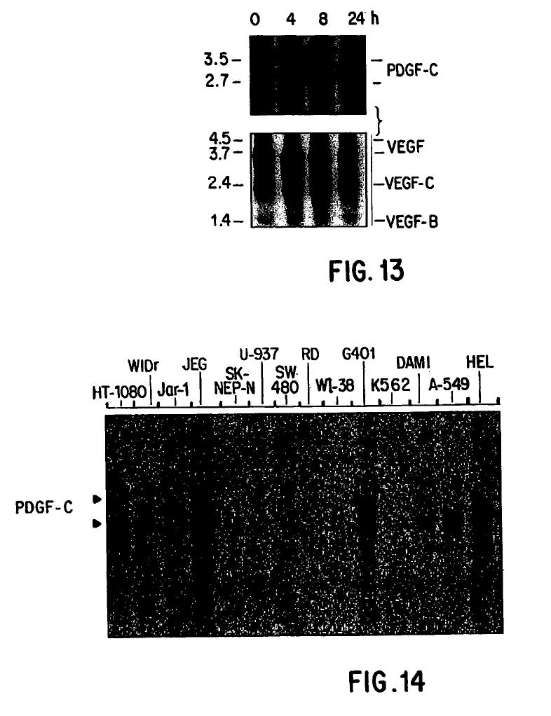

FIG. 13 shows the regulation of PDGF-C mRNA expression by hypoxia. Size markers (in kb) are indicated to the left in the lower panel. The estimated sizes of PDGF-C mRNAs is indicated to the left in the upper panel (2.7 and 3.5 kbs, respectively). To explore whether PDGF-C is induced by hypoxia, cultured human skin fibroblasts were exposed to hypoxia for 0, 4, 8 and 24 hours. Poly(A)+ mRNA was isolated from cells using oligo-dT cellulose affinity purification. Isolated mRNAs were electrophoresed through 12% agarose gels using 4 μg of mRNA per line. A Northern blot was made and hybridized with a probe for PDGF-C. The sizes of the two bands were determined by hybridizing the same filter with a mixture of hVEGF, hVEGF-B and hVEGF-C probes (Enholm et al. Oncogene, 1997 14 2475-2483), and interpolating on the basis of the known sizes of these mRNAs. The results shown in FIG. 13 indicate that PDGF-C is not regulated by hypoxia in human skin fibroblasts.

FIG. 14 shows the expression of PDGF-C mRNA in human tumor cells lines. To explore whether PDGF-C was expressed in human tumor cell lines, poly(A)+ mRNA was isolated from several known tumor cell lines, the mRNAs were electrophoresed through a 12% agarose gel and analyzed by Northern blotting and hybridization with the PDGF-C probe. The results shown in FIG. 14 demonstrate that PDGF-C mRNA is expressed in several types of human tumor cell lines such as JEG3 (a human choriocarcinoma, ATCC #HTB-36), G401 (a Wilms tumor, ATCC #CRL-1441), DAMI (a megakaryoblastic leukemia), A549 (a human lung carcinoma, ATCC #CCL-185) and HEL (a human erythroleukemia, ATCC #TID-180). It is contemplated that further growth of these PDGF-C expressing tumors can be inhibited by inhibiting PDGF-C, as well as using PDGF-C expression as a means of identifying specific types of tumors.

### EXAMPLE 1

### Generation of Specific Antipeptide Antibodies to Human PDGF-C

Two synthetic peptides were generated and then used to raise antibodies against human PDGF-C. The first synthetic peptide corresponds to residues 29-48 of the N-terminus of full length PDGF-C and includes an extra cysteine residue at the N- and C-terminus: CKFQFSSNKEQNGVQDPQHERC (SEQ ID NO:31). The second synthetic peptide corresponds to residues 230-250 of the internal region of full length PDGF-C and includes an extra cysteine residue at the C-terminus: GRKSRVVDLNLLTEEVRLYSC (SEQ ID NO:32). The two peptides were each conjugated to the carrier protein keyhole limpet hemocyanin (KLH, Calbiochem) using N-succinimidyl 3-(2-pyridyldithio)propionate (SPDP) (Pharmacia Inc.) according to the instructions of the supplier. 200-300 micrograms of the conjugates in phosphate buffered saline (PBS) were separately emulsified in Freunds Complete Adjuvant and injected subcutaneously at multiple sites in rabbits. The rabbits were boostered subcutaneously at biweekly intervals with the same amount of the conjugates emulsified in Freunds Incomplete Adjuvant. Blood was drawn and collected from the rabbits. The sera were prepared using standard procedures known to those skilled in the art.

### EXAMPLE 2

### Expression of Full Length Human PDGF-C in Mammalian Cells

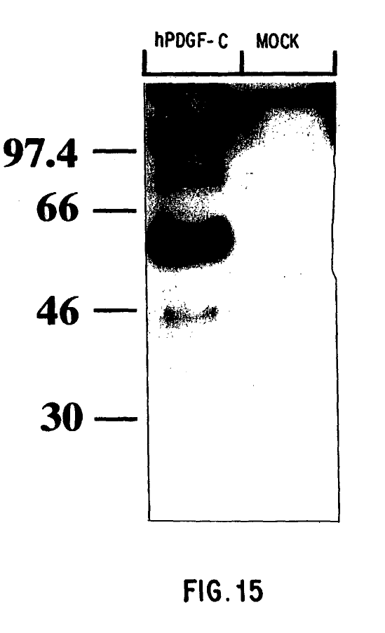

The full length cDNA encoding human PDGF-C was cloned into the mammalian expression vector, pSG5 (Stratagene, La Jolla, Calif.) that has the SV40 promoter. COS-1 cells were transfected with this construct and in separate transfections, with a pSG5 vector without the cDNA insert for a control, using the DEAE-dextran procedure. Serum free medium was added to the transfected COS-1 cells 24 hours after the transfections and aliquots containing the secreted proteins were collected for a 24 hour period after the addition of the medium. These aliquots were subjected to precipitation using ice cold 10% trichloroacetic acid for 30 minutes, and the precipitates were washed with acetone. The precipitated proteins were dissolved in SDS loading buffer under reducing conditions and separated on a SDS-PAGE gel using standard procedures. The separated proteins were electrotransferred onto Hybond filter and immunoblotted using a rabbit antiserum against the internal peptide of full length PDGF-C, the preparation of which is described above. Bound antibodies were detected using enhanced chemiluminescence (ECL, Amersham Inc.). FIG. 15 shows the results of this immunoblot. The sample was only partially reduced and the monomer of the human PDGF-C migrated as a 55 kDa species (the lower band) and the dimer migrated as a 100 kDa species (upper band). This indicates that the protein is secreted intact and that no major proteolytic processing occurs during secretion of the molecule in mammalian cells. Example 3: Expression of full length and truncated human PDGF-C in baculovirus infected Sf9 cells.

The full length coding part of the human PDGF-C cDNA (970 bp) was amplified by PCR using Deep Vent DNA polymerase (Biolabs) using standard conditions and procedures. The full length PDGF-C was amplified for 30 cycles, where each cycle consisted of one minute denaturization at 94° C., one minute annealing at 56° C. and two minutes extension at 72° C. The forward primer used was 5′CGGGATCCCGAATCCAACCTGAGTAG3′ (SEQ ID NO:33). This primer includes a BamHI site (underlined) for in frame cloning. The reverse primer used was:

| | |
|---|---|
| 5′GGAATTCCTAATGGTGATGGTGATGATGTTTGTCATCGTCATCTCCTCCTG | (SEQ ID NO:34) |
| TGCTCCCTCT3′. | |

This primer includes an EcoRI site (underlined) and sequences coding for a C-terminal 6×His tag preceded by an enterokinase site. In addition, residues 230-345 of the PDGF/VEGF homology domain (PVHD) i.e. The core domain protein of human PDGF-C were amplified by PCR using Deep Vent DNA polymerase (Biolabs) using standard conditions and procedures. The residues 230-345 of the PVHD of PDGF-C were amplified for 25 cycles, where each cycle consisted of one minute denaturization at 94° C., four minutes annealing at 56° C. and four minutes extension at 72° C. The forward primer used was 5′CGGATCCCGGAAGAAAATCCA GAGTGGTG3′ (SEQ ID NO:35).

This primer includes a BamHI site (underlined) for in frame cloning. The reverse primer used was

| | |
|---|---|
| 5′GGAATTCCTAATGGTGATGGTGATGATGTTTGTCATCGTCATCTCCTCCTG | (SEQ ID NO:36) |
| TG CTCCCTCT-3′. | |

This primer includes an EcoRI site (underlined) and sequences coding for a C-terminal 6× His tag preceded by an enterokinase site. The PCR products were digested with BamHI and EcoRI and subsequently cloned into the baculovirus expression vector, pAcGP67A. Verification of the correct sequence of the PCR products cloned into the constructs was by nucleotide sequencing. The expression vectors were then co-transfected with BaculoGold linearized baculovirus DNA into Sf9 insect cells according to the manufactures protocol (Pharmingen). Recombined baculovirus were amplified several times before beginning large scale protein production and protein purification according to the manual (Pharmingen).

Sf9 cells, adapted to serum free medium, were infected with recombinant baculovirus at a multiplicity of infection of about 7. Media containing the recombinant proteins were harvested 4 days after infection and were incubated with Ni-NTA-Agarose beads (Qiagen). The beads were collected in a column and after extensive washing with 50 mM sodium phosphate buffer pH 8, containing 300 mM NaCl (the washing buffer), the bound proteins were eluted with increasing concentrations of imidazole (from 100 mM to 500 mM) in the washing buffer. The eluted proteins were analyzed by SDS-PAGE using 12.5% polyacrylamide gels under reducing and non-reducing conditions. For immunoblotting analyses, the proteins were electrotransferred onto Hybond filters for 45 minutes.

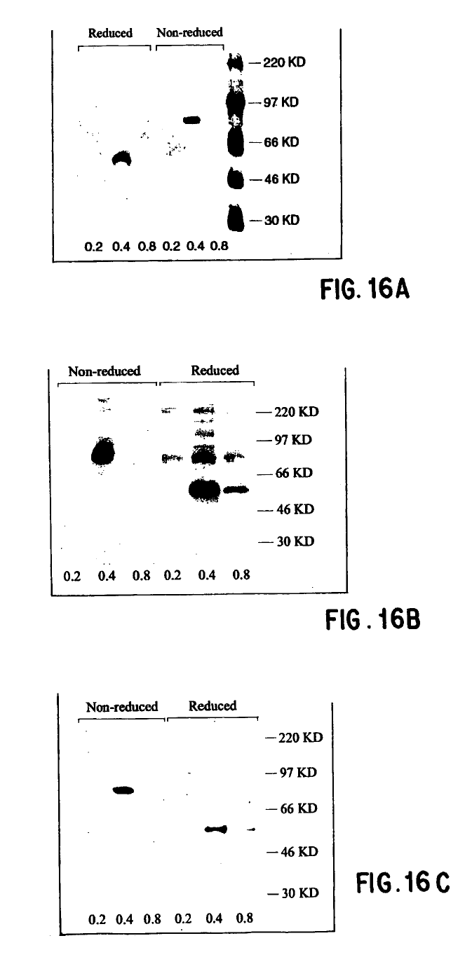

FIGS. 16A-C show the isolation and partial characterization of full length human PDGF-C protein. In FIG. 16A, the recombinant full length protein was visualized on the blot using antipeptide antibodies against the N-terminal peptide (described above). In FIG. 16B, the recombinant full length protein was visualized on the blot using antipeptide antibodies against the internal peptide (described above). The separated proteins were visualized by staining with Coomassie Brilliant Blue (FIG. 16C). The numbers at the bottom of FIGS. 16A-C refer to the concentration of imidazole used to elute the protein from the Ni-NTA column and are expressed in molarity (M). FIGS. 16A-C also show that the full length protein migrates as a 90 kDa species under non-reducing conditions and as a 55 kDa species under reducing conditions. This indicates that the full length protein was expressed as a disulfide-linked dimer.

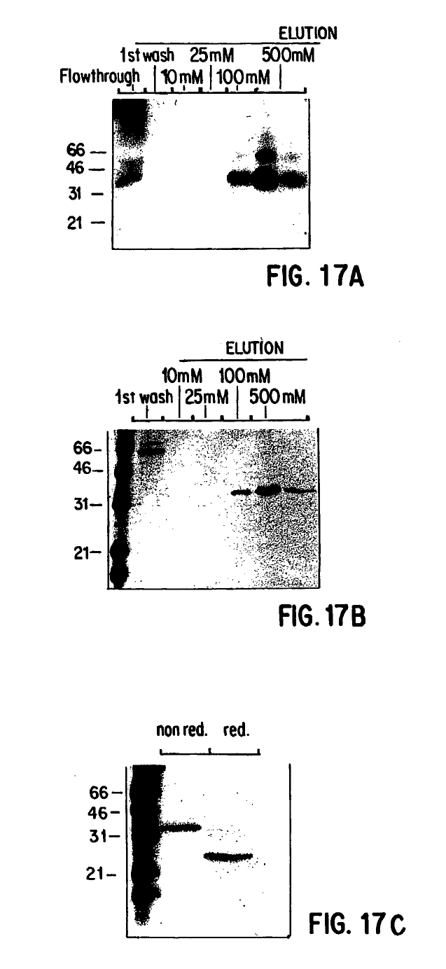

FIGS. 17A-C show the analysis of the isolation and partial characterization of a truncated form of human PDGF-C containing the PDGF/VEGF homology domain only. In FIG. 17A, the immunoblot analysis of fractions eluted from the Ni-agarose column demonstrates that the protein could be eluted at imidazole concentrations ranging between 100-500 mM. The eluted fractions were analyzed under non-reducing conditions, and the truncated human PDGF-C was visualized on the blot using antipeptide antibodies against the internal peptide (described above). FIG. 17B shows the Coomassie Brilliant Blue staining of the same fractions as in FIG. 17A. This shows that the procedure generates highly purified material migrating as a 36 kDa species. FIG. 17C shows the Coomassie Brilliant Blue staining of non-reduced (non-red.) and reduced (red.) truncated human PDGF-C protein. The data show that the protein is a secreted dimer held together by disulfide bonds and that the monomer migrates as a 24 kDa species.

### EXAMPLE 4

### Receptor Binding Properties of Full Length and Truncated PDGF-C

To assess the interactions between full length and truncated PDGF-C and the VEGF receptors, full length and truncated PDGF-C were tested for their capacity to bind to soluble Ig-fusion proteins containing the extracellular domains of human VEGFR-1, VEGFR-2 and VEGFR-3 (Olofsson et al., Proc. Natl. Acad. Sci. USA, 1998 95:11709-11714). The fusion proteins, designated VEGFR-1-Ig, VEGFR-2-Ig and VEGFR-3-Ig, were transiently expressed in human 293 EBNA cells. All Ig fusion proteins were human VEGFRs. Cells were incubated for 24 hours after transfection, washed with Dulbecco's Modified Eagle Medium (DMEM) containing 0.2% bovine serum albumin and starved for 24 hours. The fusion proteins were then precipitated from the clarified conditioned medium using protein A-Sepharose beads (Pharmacia). The beads were combined with 100 microliters of 10× binding buffer (5% bovine serum albumin, 0.2% Tween 20 and 10 μg/ml heparin) and 900 microliter of conditioned medium from 293 cells that had been transfected with mammalian expression plasmids encoding full length or truncated PDGF-C or control vector, then metabolically labeled with ³⁵S-cysteine and methionine (Promix, Amersham) for 4 to 6 hours. After 2.5 hours, at room temperature, the Sepharose beads were washed 3 times with binding buffer at 4° C., once with phosphate buffered saline and boiled in SDS-PAGE buffer. Labeled proteins that were bound to the Ig-fusion proteins were analyzed by SDS-PAGE under reducing conditions. Radiolabeled proteins were detected using a phosphorimager analyzer. In all these analyses, radiolabeled PDGF-C failed to show any interaction with any of the VEGF receptors.

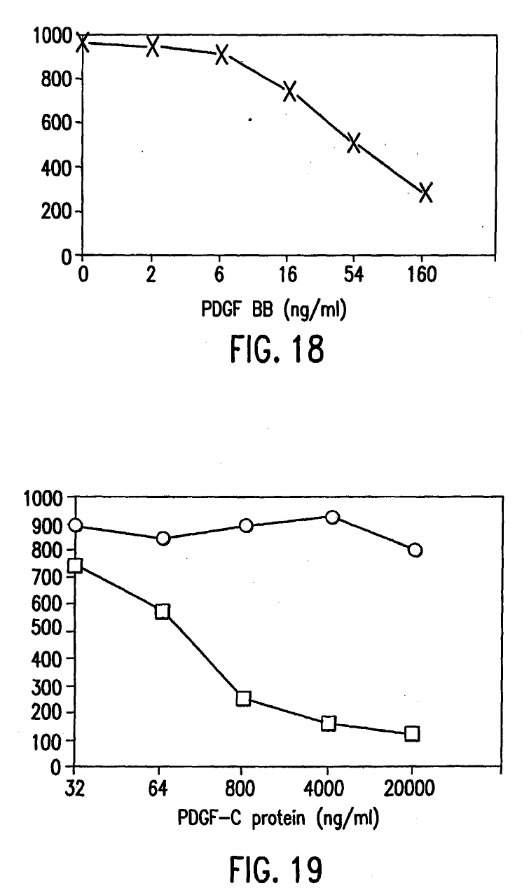

Next, full length and truncated PDGF-C were tested for their capacity to bind to human PDGF receptors alpha and beta by analyzing their abilities to compete with PDGF-BB for binding to PDGF receptors. The binding experiments were performed on porcine aortic endothelial-1 (PAE-1) cells stably expressing the human PDGF receptors alpha and beta (Eriksson et al., EMBO J, 1992, 11, 543-550). Binding experiments were performed essentially as in Heldin et al. (EMBO J, 1988, 7:1387-1393). Different concentrations of human full-length and truncated PDGF-C, or human PDGF-BB were mixed with 5 ng/ml of ¹²⁵I-PDGF-BB in binding buffer (PBS containing 1 mg/ml of bovine serum albumin). Aliquots were incubated with the receptor expressing PAE-1 cells plated in 24-well culture dishes on ice for 90 minutes. After three washes with binding buffer, cell-bound ¹²⁵I -PDGF-BB was extracted by lysis of cells in 20 mM Tris-HCl, pH 7.5, 10% glycerol, 1% Triton X-100. The amount of cell bound radioactivity was determined in a gamma-counter. A standard curve for the binding of ¹²⁵I-labeled PDGF BB homodimers to PAE-1 cells expressing PDGF alpha-receptor is shown in FIG. 18. An increasing excess of the unlabeled protein added to the incubations competed efficiently with cell association of the radiolabeled tracer.

FIG. 19 graphically shows that the truncated PDGF-C efficiently competed for binding to the PDGF alpha-receptor, while the full length protein did not. Both the full length and truncated proteins failed to compete for binding to the PDGF beta-receptor.

### EXAMPLE 5

### PDGF Alpha-Receptor Phosphorylation

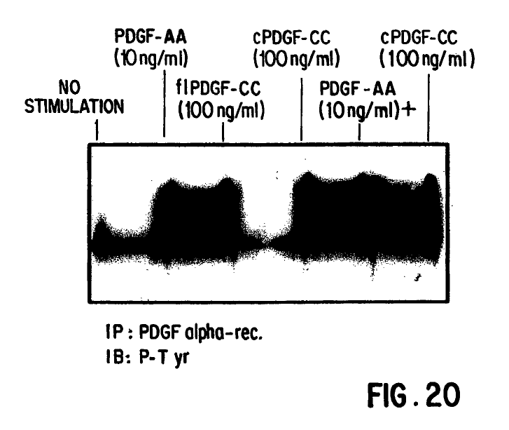

To test if PDGF-C causes increased phosphorylation of the PDGF alpha-receptor, full length and truncated PDGF-C were tested for their capacity to bind to the PDGF alpha-receptor and stimulate increased phosphorylation. Serum-starved porcine aortic endothelial (PAE) cells stably expressing the human PDGF alpha-receptor were incubated on ice for 90 minutes with PBS supplemented with 1 mg/ml BSA and 10 ng/ml of PDGF-AA, 100 ng/ml of full length human PDGF-CC homodimers (flPDGF-CC), 100 ng/ml of truncated PDGF-CC homodimers (cPDGF-CC), or a mixture of 10 ng/ml of PDGF-AA and 100 ng/ml of truncated PDGF-CC. Full length and truncated PDGF-CC homodimers were produced as described above. Sixty minutes after the addition of the polypeptides, the cells were lysed in lysis buffer (20 mM tris-HCl, pH 7.5, 0.5% Triton X-100, 0.5% deoxycholic acid, 10 mM EDTA, 1 mM orthovanadate, 1 mM PMSF 1% Trasylol). The PDGF alpha-receptors were immunoprecipitated from cleared lysates with rabbit antisera against the human PDGF alpha-receptor (Eriksson et al., EMBO J, 1992 11:543-550). The precipitated receptors were applied to a SDS-PAGE gel. After SDS gel electrophoresis, the precipitated receptors were transferred to nitrocellulose filters, and the filters were probed with anti-phosphotyrosine antibody PY-20, (Transduction Laboratories). The filters were then incubated with horseradish peroxidase-conjugated anti-mouse antibodies. Bound antibodies were detected using enhanced chemiluminescence (ECL, Amersham Inc). The filters were then stripped and reprobed with the PDGF alpha-receptor rabbit antisera, and the amount of receptors was determined by incubation with horseradish peroxidase-conjugated anti-rabbit antibodies. Bound antibodies were detected using enhanced chemiluminescence (ECL, Amersham Inc). The probing of the filters with PDGF alpha-receptor antibodies confirmed that equal amounts of the receptor were present in all lanes. PDGF-AA is included in the experiment as a control. FIG. 20 shows that truncated, but not full length PDGF-CC, efficiently induced PDGF alpha-receptor tyrosine phosphorylation. This indicates that truncated PDGF-CC is a potent PDGF alpha-receptor agonist.

### EXAMPLE 6

### Mitogenicity of PDGF-C for Fibroblasts

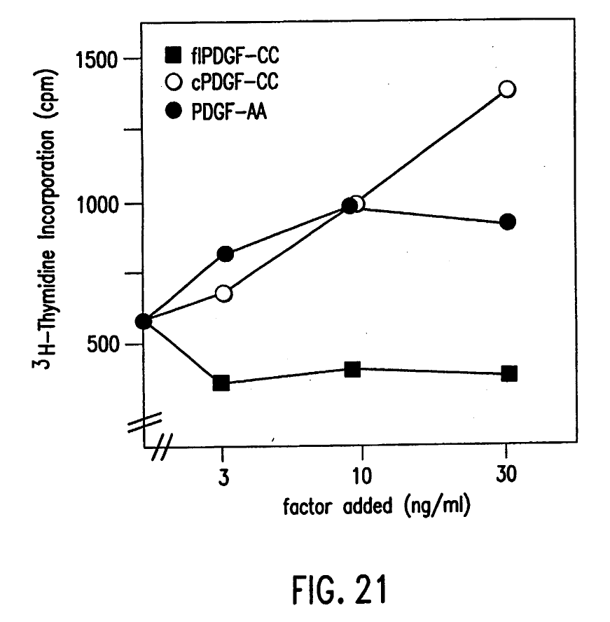

FIG. 21 shows the mitogenic activities of truncated and full length PDGF-CC on fibroblasts. The assay was performed essentially as described in Mori et al., J. Biol. Chem., 1991 266:21158-21164. Serum starved human foreskin fibroblasts were incubated for 24 hours with 1 ml of serum-free medium supplemented with 1 mg/ml BSA and 3 ng/ml, 10 ng/ml or 30 ng/ml of full length PDGF-CC (flPDGF-CC), truncated PDGF-CC (cPDGF-CC) or PDGF-AA in the presence of 0.2 μmCi [3H]thymidine. After trichloroacetic acid (TCA) precipitation, the incorporation of [3H]thymidine into DNA was determined using a beta-counter. The results show that truncated PDGF-CC, but not full length PDGF-CC, is a potent mitogen for fibroblasts. PDGF-AA is included in the experiment as a control.

PDGF-C does not bind to any of the known VEGF receptors. PDGF-C is the only VEGF family member, thus far, which can bind to and increase phosphorylation of the PDGF alpha-receptor. PDGF-C is also the only VEGF family member, thus far, to be a potent mitogen of fibroblasts. These characteristics indicate that the truncated form of PDGF-C may not be a VEGF family member, but instead a novel PDGF. Furthermore, the full length protein is likely to be a latent growth factor that needs to be activated by proteolytic processing to release the active PDGF/VEGF homology domain. A putative proteolytic site is the dibasic motif found in residues 231-234 in the full length protein, residues -R-K-S-R-. This site is structurally conserved in a comparison between mouse and human PDGF-Cs (FIG. 7). Preferred proteases include, but are not limited to, Factor X and enterokinase. The N-terminal CUB domain may be expressed as an inhibitory domain which might be used to localize this latent growth factor in some extracellular compartment (for example the extracellular matrix) and which is removed by limited proteolysis when need, for example during embryonic development, tissue regeneration, tissue remodelling including bone remodelling, active angiogenesis, tumor progression, tumor invasion, metastasis formation and/or wound healing.

### EXAMPLE 7

### PDGF Receptors Binding of Truncated PDGF-C

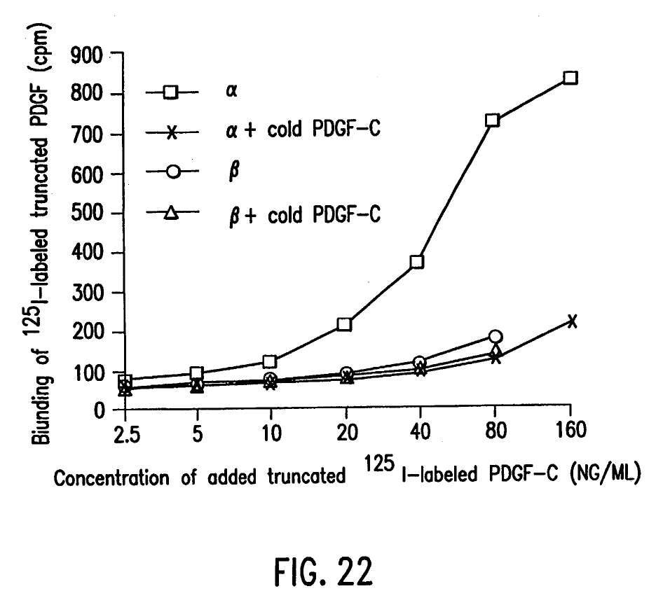

To assess the interactions between truncated PDGF-C and the PDGF alpha and beta receptors, truncated PDGF-C was tested for its capacity to bind to porcine aortic endothelial-1 (PAE-1) cells expressing PDGF alpha or beta receptors, respectively (Eriksson et al., EMBO J, 1992 11:543-550). The binding experiments were performed essentially as described in Heldin et al. (EMBO J, 1988 7:1387-1393). Five micrograms of truncated PDGF-C protein in ten microliters of sodium borate buffer was radiolabeled using the Bolton-Hunter reagent (Amersham) to a specific activity of 4×10⁵ cpm/ng. Different concentrations of radiolabeled truncated PDGF-C, with or without added unlabeled protein, in binding buffer (PBS containing 1 mg/ml of bovine serum albumin) was added to the receptor expressing PAE-1 cells plated in 24-well culture dishes on ice for 90 minutes. After three washes with binding buffer, cell-bound ¹²⁵I-labeled PDGF-C was extracted by lysis of cells in 20 mM Tris-HCl, pH 7.5, 10% glycerol, 1% Triton X-100. The amount of cell-bound radioactivity was determined in a gamma-counter. Non-specific binding was estimated by including a 100-fold molar excess of truncated PDGF-C in some experiments. All binding data represents the mean of triplicate analyses and the experimental variation in the experiment varied between 10-15%. As seen in FIG. 22, truncated PDGF-C binds to cells expressing PDGF alpha receptors, but not to beta receptor expressing cells. The binding was specific as radiolabeled PDGF-C was quantitatively displaced by a 100-fold molar excess of unlabeled protein.

### EXAMPLE 8

### Protease Effects on Full Length PDGF-C

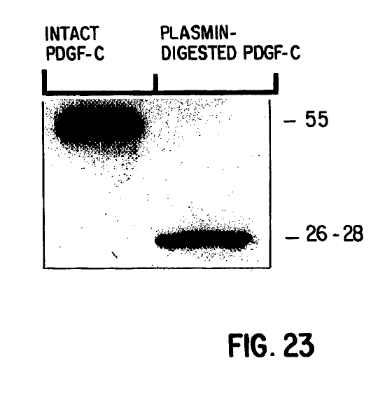

To demonstrate that full length PDGF-C can be activated by limited proteolysis to release the PDGF/VEGF homology domain from the CUB domain, the full length protein was digested with different proteases. For example, full length PDGF-C was digested with plasmin in 20 mM Tris-HCl (pH 7.5) containing 1 mM CaCl₂, 1 mM MgCl2 and 0.01% Tween 20 for 1.5 to 4.5 hours at 37° C. using two to three units of plasmin (Sigma) per ml. The released domain essentially corresponded in size to the truncated PDGF-C species previously produced in insect cells. Plasmin-digested PDGF-C and undigested full length PDGF-C were applied to a SDS-PAGE gel under reducing conditions. After SDS-PAGE gel electrophoresis, the respective proteins were transferred to a nitrocellulose filter, and the filter was probed using a rabbit antipeptide antiserum to residues 230-250 in full length protein (residues GRKSRVVDLNLLTEEVRLYSC (SEQ ID NO:37) located in just N-terminal to the PDGF/VEGF homology domain). Bound antibodies were detected using enhanced chemiluminescence (ECL, Amersham Inc). FIG. 23 shows the immunoblot with a 55 kDa undigested full length protein and the plasmin-generated 26-28 kDa species.

### EXAMPLE 9

### PDGF alpha Receptors Binding of Plasmin-Digested PDGF-C

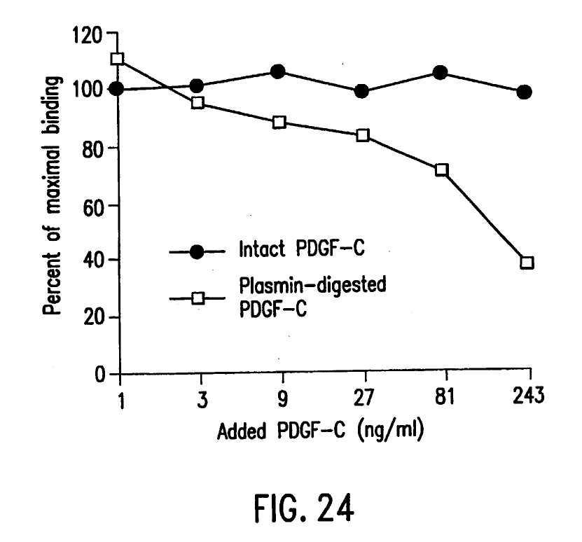

To assess the interactions between plasmin-digested PDGF-C and the PDGF alpha receptors, plasmin-digested PDGF-C was tested for its capacity to bind to porcine aortic endothelial-1 (PAE-1) cells expressing PDGF alpha receptors (Eriksson et al., EMBO J, 1992 11:543-550). The receptor binding analyses were performed essentially as in Example 7 using 30 ng/ml of ¹²⁵I-labeled truncated PDGF-C as the tracer. As seen in FIG. 24, increasing concentrations of plasmin-digested PDGF-C efficiently competed for binding to the PDGF alpha receptors. In contrast, undigested full length PDGF-C failed to compete for receptor binding. These data indicate that full length PDGF-C is a latent growth factor unable to interact with PDGF alpha receptors and that limited proteolysis, which releases the C-terminal PDGF/VEGF homology domain, is necessary to generate an active PDGF alpha receptor ligand/agonist.

### EXAMPLE 10

### Cloning and Expression of the Human PDGF-C CUB Domain

A human PDGF-C 430 bp cDNA fragment encoding the CUB domain (amino acid residues 23-159 in full length PDGF-C) was amplified by PCR using Deep Vent DNA polymerase (Biolabs) using standard conditions and procedures. The forward primer used was

5′-CGGATCCCGAATCCAACCTGAGTAG-3′ (SEQ ID NO:38).

This primer includes a BamHI site (underlined) for in clone frame cloning. The reverse primer used was

| | |
|---|---|
| 5′-CCGGAATTCCTAATGGTGATGGTGATGATGTTTGTCATCGTCGTCGA-CAATGTTGTAGTG-3′. | (SEQ ID NO:39) |

This primer includes an EcoRI site (underlined) and sequences coding for a C-terminal 6×His tag preceded by an enterokinase site. The amplified PCR fragment was subsequently cloned into a pACgp67A transfer vector. Verification of the correct sequence of the expression construct, CUB-pACgp67A, was by automatic nucleotide sequencing. The expression vectors were then co-transfected with BaculoGold linearized baculovirus DNA into Sf9 insect cells according to the manufacture's protocol (Pharmingen). Recombined baculovirus were amplified several times before beginning large scale protein production and protein purification according to the manual (Pharmingen).

Sf9 cells, adapted to serum free medium, were infected with recombinant baculovirus at a multiplicity of infection of about 7. Media containing the recombinant proteins were harvested 72 hours after infection and were incubated with Ni-NTA-Agarose beads (Qiagen) overnight at 4° C. The beads were collected in a column and after extensive washing with 50 mM sodium phosphate buffer pH 8, containing 300 mM NaCl (the washing buffer), the bound proteins were eluted with increasing concentrations of imidazole (from 100 mM to 400 mM) in the washing buffer. The eluted proteins were analyzed by SDS-PAGE using a polyacrylamide gel under reducing and non-reducing conditions.

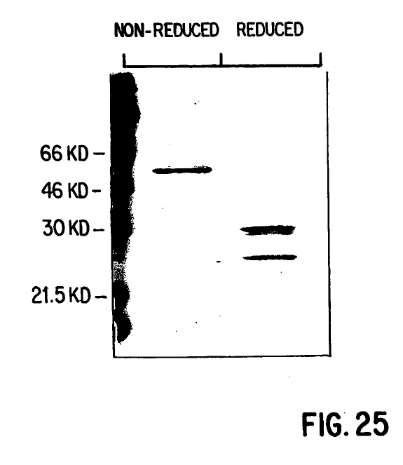

FIG. 25 shows the results from Coomassie blue staining of the gel. The human PDGF-C CUB domain is a disulfide-linked homodimer with a molecular weight of about 55 KD under non-reducing conditions, while two monomers of about 25 and 30 KD respectively are present under reducing conditions. The heterogeneity is probably due to heterogenous glycosylation of the two putative N-linked glycosylation sites present in the CUB domain at amino acid positions 25 and 55. A protein marker lane is shown to the left in the figure.

### EXAMPLE 11

### Localization of PDGF-C Transcripts in Developing Mouse Embryos

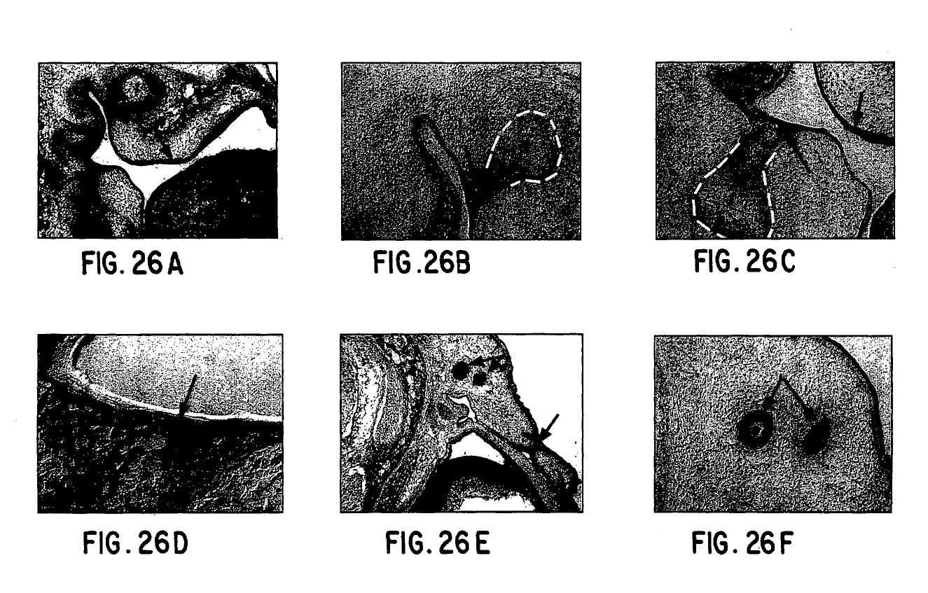

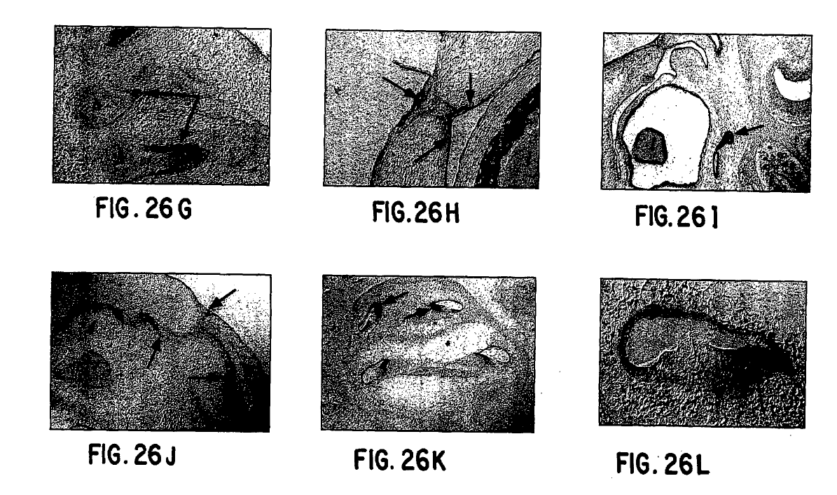

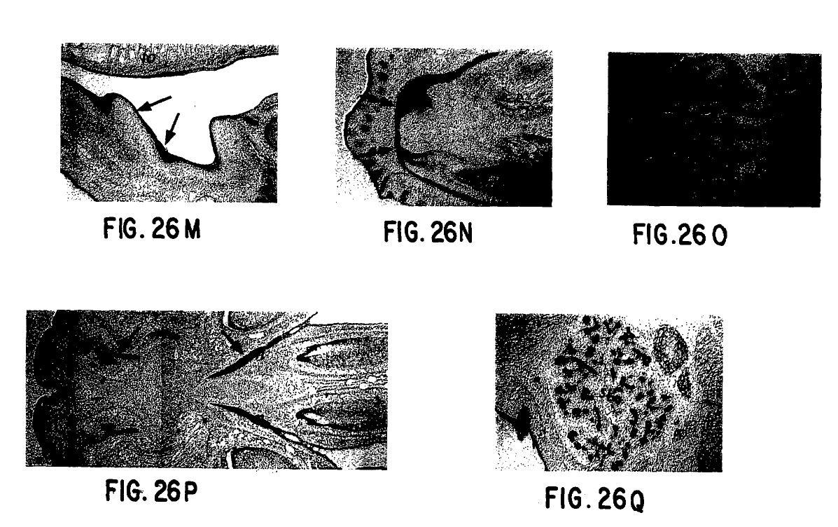

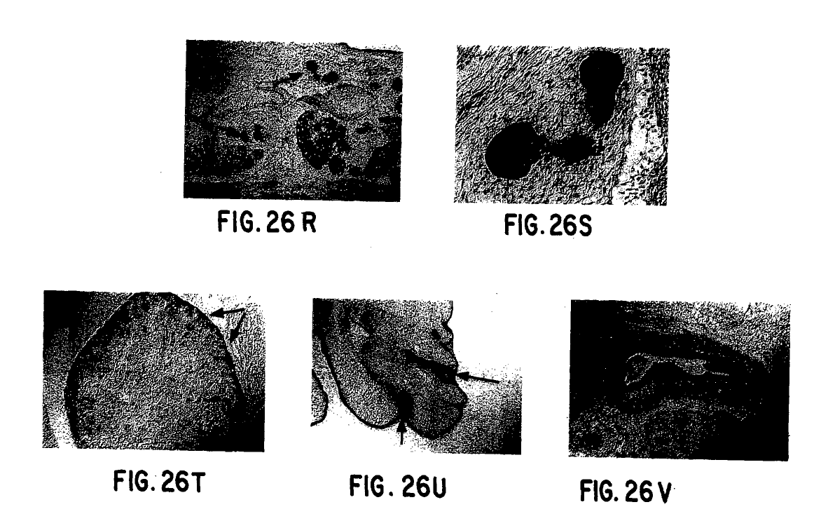

To gain insight into the biological function of PDGF-C, PDGF-C expression in mouse embryos was localized by non-radioactive in situ hybridization in tissue sections from the head (FIGS. 26A-26S) and urogenital tract (FIGS. 26T-26V) regions. The non-radioactive in situ hybridization employed protocols and PDGF-A and PDGFR-alpha probes are described in Boström et al., Cell, 1996 85:863-873, which is hereby incorporated by reference. The PDGF-C probe was derived from a mouse PDGF-C cDNA. The hybridization patterns shown in FIGS. 26A-26V are for embryos aged E16.5, but analogous patterns are seen at E14.5, E15.5 and E17.5. Sense probes were used as controls and gave no consistent pattern of hybridization to the sections.

FIG. 26A shows the frontal section through the mouth cavity at the level of the tooth anlagen (t). The arrows point to sites of PDGF-C expression in the oral ectoderm. Also shown is the tongue (to). FIGS. 26B-26D show PDGF-C expression in epithelial cells of the developing tooth canal. Individual cells are strongly labeled in this area (arrow in FIG. 26D), as well as in the developing palate ectoderm (right arrow in FIG. 26C). FIG. 26E shows the frontal section through the eye, where PDGF-C expression is seen in the hair follicles (double arrow) and in the developing eyelid. Also shown is the retina (r). In FIGS. 26F and 26G, the PDGF-C expression is found in the outer root sheath of the developing hair follicle epithelium. In FIG. 26H, PDGF-C expression is shown in the developing eyelid. There is an occurrence of individual strongly PDGF-C positive cells in the developing opening. Also shown is the lens (l). In FIG. 261, PDGF-C expression in the developing lacrimal gland is shown by the arrow. In FIG. 26J, PDGF-C expression in the developing external ear is shown. Expression is seen in the external auditory meatus (left arrow) and in the epidermal cleft separating the prospective auricle (e). FIGS. 26K and 26L show PDGF-C expression in the cochlea. Expression is seen in the semi-circular canals (arrows in 26K). There is a polarized distribution of PDGF-C mRNA in epithelial cells adjacent to the developing hair cells (arrow in 26L). FIGS. 26M and 26N show PDGF-C expression in the oral cavity. Horizontal sections show expression in buccal epithelium (arrows in 26M) and in the forming cleft between the lower lip buccal and the gingival epithelium (arrows in 26N). Also shown is the tooth anlagen (t) and the tongue (to). FIGS. 26O and 26P show PDGF-C expression in the developing nostrils, shown on horizontal sections. PDGF-C expression appears strongest before stratification of the epithelium and the formation of the canal proper (arrows in 260 and 26P). Also shown is the developing nostrils (n). FIGS. 26Q-26S show PDGF-C expression in developing salivary glands and ducts. FIG. 26Q is the sublingual gland. FIGS. 26R and 26S show the maxillary glands, the salivary gland (sg) and the salivary duct (sd). FIGS. 26T-26V show the expression of PDGF-C in the urogenital tract. FIG. 26T shows the expression of PDGF-C in the developing kidney metanephric mesoderm. FIG. 26U shows the expression of PDGF-C in the urethra (ua) and in epithelium surrounding the developing penis. FIG. 26V shows the PDGF-C expression in the developing ureter (u).

### EXAMPLE 12

### PDGF-C, PDGF-A and PDGFR-alpha Expression in the Developing Kidney

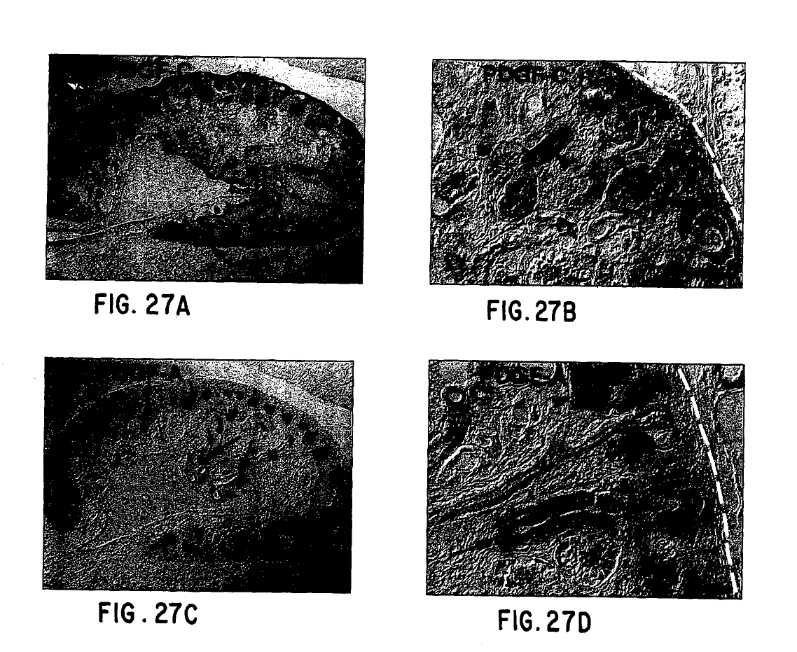

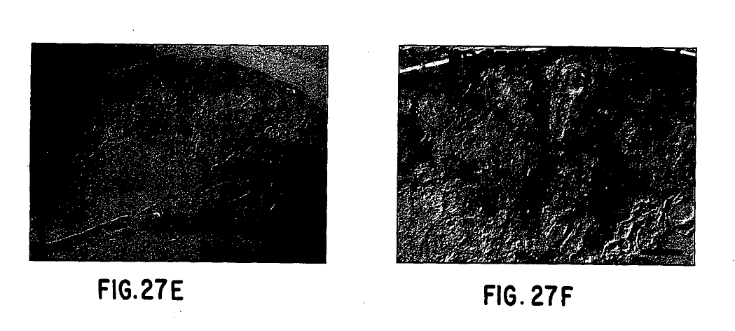

One of the strongest sites of PDGF-C expression is the developing kidney and so expression of PDGF-C, PDGF-A and PDGFR-alpha was looked at in the developing kidney. FIGS. 27A-27F show the results of non-radioactive in situ hybridization demonstrating the expression (blue staining in unstained background visualized using DIC optics) of mRNA for PDGF-C (FIGS. 27A and 27B), PDGF-A (FIGS. 27C and 27D) and PDGFR-alpha (FIGS. 27E and 27F) in E16.5 kidneys. The white hatched line in FIGS. 27B, 27D and 27F outlines the cortex border. The bar in FIGS. 27A, 27C and 27E represents 250 μm, and in FIGS. 27B, 27D and 27F represents 50 μm.

PDGF-C expression is seen in the metanephric mesenchyme (mm in FIG. 27A), and appears to be upregulated in the condensed mesenchyme (arrows in FIG. 27B) undergoing epithelial conversion as a prelude to tubular development, which is situated on each side of the ureter bud (ub). PDGF-C expression remains at lower levels in the early nephronal epithelial aggregates (arrowheads in B), but is absent from mature glomeruli (gl) and tubular structures.

PDGF-A expression is not seen in these early aggregates, but is strong in later stages of tubular development (FIGS. 24C and 24D). PDGF-A is expressed in early nephronal epithelial aggregates (arrowheads in FIG. 27D), but once the nephron is developed further, PDGF-A expression becomes restricted to the developing Henle's loop (arrow in FIG. 27D). The strongest expression is seen in the Henle's loops in the developing marrow (arrows in FIG. 27C). The branching ureter (u) and the ureter bud (ub) is negative for PDGF-A.

Thus, the PDGF-C and PDGF-A expression patterns in the developing nephron are spatially and temporally distinct. PDGF-C is expressed in the earliest stages (mesenchymal aggregates) and PDGF-A in the latest stages (Henle's loop formation) of nephron development.

PDGFR-alpha is expressed throughout the mesenchyme of the developing kidney (FIGS. 27E and 27F) and may hence be targeted by both PDGF-C and PDGF-A. PDGF-B expression is also seen in the developing kidney, but occurs only in vascular endothelial cells. PDGFR-beta expression takes place in perivascular mesenchyme, and its activation by PDGF-B is critical for mesangial cell recruitment into glomeruli.

These results demonstrate that PDGF-C expression occurs in close spatial relationship to sites of PDGFR-alpha expression, and are distinct from the expression sites of PDGF-A or PDGF-B. This indicates that PDGF-C may act through PDGFR-alpha in vivo, and may have functions that are not shared with PDGF-A and PDGF-B.

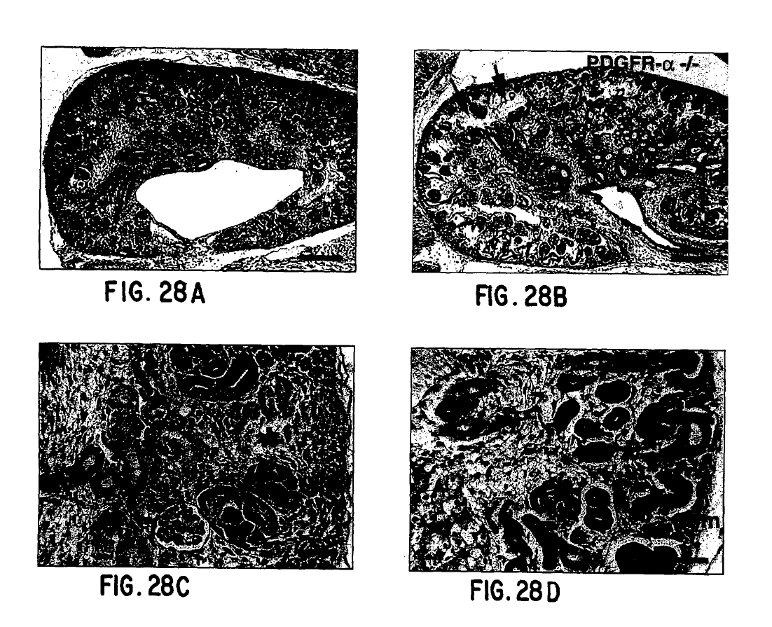

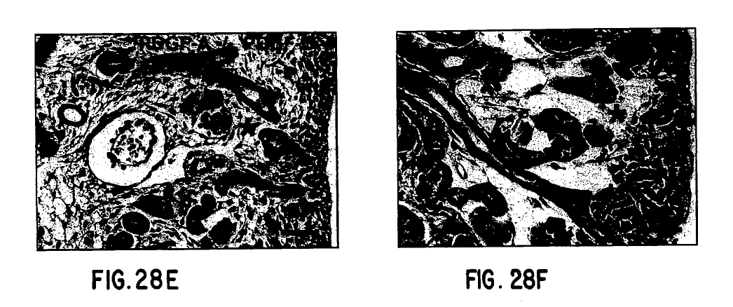

Since the unique expression pattern of PDGF-C in the developing kidney indicates a function as a PDGFR-alpha agonist separate from that of PDGF-A or -B, a comparison was made to the histology of embryonic day 16.5 kidneys from PDGFR-alpha knockout mice (FIGS. 28B and 28F) with kidneys from wildtype (FIGS. 28A and 28C), PDGF-A knockout (FIG. 28D) and PDGF-A/PDGF-B double knockout (FIG. 28E) mice. The bar in FIGS. 28A and 28B represents 250 μm, and in FIGS. 28C-28F represents 50 μm.

Heterozygote mutants of PDGF-A, PDGF-B and PDGFR-alpha (Boström et al., Cell, 1996 85:863-873; Levéen et al., Genes Dev., 1994 8:18751887; Soriano et al., Development, 1997 124:2691-70) were bred as C57B16/129sv hybrids and intercrossed to produce homozygous mutant embryos. PDGF-A/PDGF-B heterozygote mutants were crossed to generate double PDGF-A/PDGF-B knockout embryos. Due to a high degree of lethality of PDGF-A −/− embryos before E10 (Boström et al., Cell, 1996 85:863-873), the proportion of double knockout E16.5 embryos obtained in such crosses were less than 1/40. The histology of kidney phenotypes was verified on at least two embryos of each genotype, except the PDGF-A/PDGF-B double knockout for which only a single embryo was obtained.

It is interesting that there is lack of interstitial mesenchyme in the cortex of PDGFR-alpha−/− kidney (arrows in FIG. 28A and asterisk in FIG. 28F) and the presence of interstitial mesenchyme in all other genotypes (asterisks in FIGS. 28C-E). The branching ureter (u) and the metanephric mesenchyme (mm) and its epithelial derivatives appear normal in all mutants. The abnormal glomerulus in the PDGF-A/PDGF-B double knockout reflect failure of mesangial cell recruitment into the glomerular tuft due to the absence of PDGF-B.

These results indicate that PDGFR-alpha knockouts have a kidney phenotype, which is not seen in PDGF-A or PDGF-A/PDGF-B knockouts, hence potentially reflecting loss of signaling by PDGF-C. The phenotype consists of the marked loss of interstitial mesenchyme in the developing kidney cortex. The cells lost in PDGFR-alpha −/− kidneys are thus normally PDGFR-alpha positive cells adjacent to the site of expression of PDGF-C.

### EXAMPLE 13

### Proteolytic Processing of PDGF-C by Human Fibroblastic 1523 Cells

Endogenous PDGF-C from human fibroblastic AG1523 cells is expressed as two principal species of about Mᵣ 25K, corresponding to processed PDGF-C, and a minor species of Mᵣ 55K, corresponding to the full-length protein. To obtain further information on the proteolytic process, serum-free medium was collected from ˜80% confluent AG1523 cells. TCA-precipitated proteins from 1 ml of medium were subjected to SDS-page using a 12% polyacrylamide gel (BioRad) under reducing conditions and then immunoblotted. Endogenous PDGF-C was detected using a rabbit anti-peptide antiserum against an internal peptide located in the human PDGF-CC core domain (Li et al., 2000). Bound antibodies were observed using enhanced chemiluminiscence Plus (ECL+; Amersham).

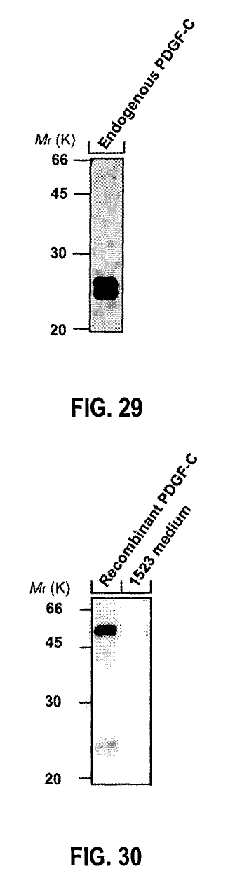

As seen in FIG. 29, two principal Mᵣ 25 kDa species can be seen, as well as a weak band of Mᵣ 55 kDa corresponding to full length PDGF-C. The results show that conditioned medium from the AG1523 fibroblasts produced proteolytic activity that will process full length PDGF-C into active and receptor-competent PDGF-C.

### EXAMPLE 14

### Expression of Recombinant Human PDGF-C in Sf9 Insect Cells

Recombinant full-length human PDGF-C was expressed in Sf9 insect cells using the baculovirus expression system (see, e.g., Example 3; and Li et al., 2000, Nat. Cell Biol. 2:302-309, incorporated herein by reference). Recombinant full-length PDGF-C is expressed as a major species of Mr 55K in baculovirus-infected Sf9 cells. Serum-free medium was collected. TCA-precipitated proteins from 0.2 ml of the medium were subjected to SDS-page using a 12% polyacrylamide gel (BioRad) under reducing conditions and then immunoblotted. The His₆-tagged PDGF-C was detected using an anti-His₆ epitope monoclonal antibody (C-terminal, InVitrogen). No protein was detected in 1523 medium with this anti-His₆ epitope monoclonal antibody. Bound antibodies were observed using enhanced chemiluminiscence Plus (ECL+; Amersham).

As seen in FIG. 30, there is a light band at about 25 K, indicating a low but nonetheless significant endogenous processing of full length PDGF-C. Further, it can be seen that His₆ epitopes in proteins in the medium are absent from AG1523 cells.

### EXAMPLE 15

### Protease Inhibitor Analysis

To elucidate the mechanism of the proteolysis of PDGF-C a protease inhibitor analysis was conducted. Various protease inhibitors (see Table 1, source: Sigma) were pre-incubated with 0.9 ml of AG1523 serum-free medium at room temperature for 30 minutes, then incubated with 0.2 ml of recombinant full-length PDGF-C (Sf9 serum-free medium) at 37° C. overnight. TCA-precipitated proteins were subjected to SDS-page under reducing conditions and then immunoblotted. Recombinant PDGF-C was detected using an anti-His₆ epitope monoclonal antibody (C-terminal) (InVitrogen).

TABLE 1
Protease inhibitors

| | | Final | |
|---|---|---|---|
| Name | Inhibitor Of | Concentration | Solvent |
| AEBSF | Serine Proteases | 1 mM | Water |
| Bestatin | Aminoprptodases | 100 μM | Water |
| Leupeptin | Serine & Cysting | 100 μM | Water |
| | Proteases | | |
| Pepstatin A | Acid Proteases | 10 μM | <1% |
| | | | DMSO |
| E64 | Cystine & Thiol Proteases | 100 μM | Water |
| Aprotinin | Serine Proteases | 100 μM | Water |
| | | (˜3TIU) | |
| EDTA | Metalloproteases | 50 μM | Water |
| Phosphoramidon | Metalloendoproteases | 100 μM | Water |

By increasing the amount of conditioned AG1523 medium and varying the co-incubated protease inhibitors, recombinant full-length PDGF-CC was cleaved in a dose-dependent manner. This indicates that the involved protease is present in the AG1523 medium and that the processing occurs extracellularly.

The serine protease inhibitors were able to decrease the proteolysis as compared to control, indicating the serine proteases are those involved in the processing of PDGF-C. In particular, Aprotinin showed a capacity to inhibit proteolytic processing, thus the serine protease is expected to be trypsin-like. Trypsin-like serine proteases are proteases containing trypsin like domains.

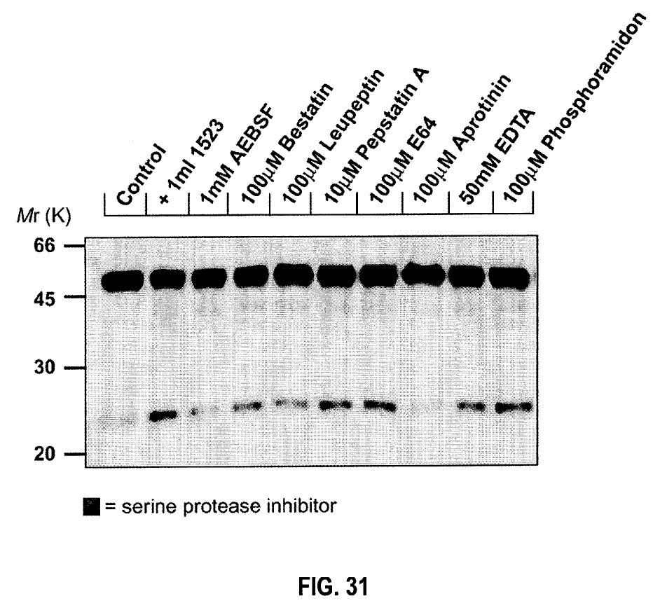

As seen in corresponding FIG. 31, conditioned medium from AG1523 fibroblasts contains a serine protease with trypsin-like properties that processes PDGF-C.

### EXAMPLE 16

### PDGF-C Promoted Revascularization following Heart Infarction

Chronic myocardial ischemia was replicated by ligation of the left anterior descending (LAD) coronary artery using anesthetized 10 week old normal C57B16 mice. For PDGF-C treatment mice, 10 μg of recombinant human PDGF-CC core domain protein produced in baculovirus infected insect cells were administered after heart infarction using a subcutaneous osmotic minipump for seven days (ALZET™ osmotic pump, DURECT Corporation, Cupertino, Calif.). Seven days after LAD ligation, infarcted hearts were fixed and collected. The PDGF-CC core domain protein (SEQ ID NO: 40) corresponds to corresponds to residues 230-345 of full-length PDGF-C protein i.e. amino acids 230-345 of SEQ ID NO:3. The hearts were sectioned longitudinally into 6 μm sections. Hematoxylin-eosine and immunohistochemical stainings were performed using thrombomodulin as a marker for endothelial cells. Smooth muscle alpha-actin was used as a marker for vascular smooth muscle cells. Infarcted areas and vessel densities were calculated using a Quantinet Q600 image analysis system (Leica, Brussels, Belgium). Data were statistically analyzed using the Student T test.

In the PDGF-CC treated mice, total vessel density was about 136% of that of the normal mice (P=0.07, 56±16.6 versus 41.2±14.2 total vessels/mm²). Values are presented as the average±SD, PDGF-CC treated mice n=6 versus normal mice n=11. The vessels were further classified into three different groups, large (>30 μm), medium (10-30 μm), and small (<10 μm). The large vessel density in PDGF-CC treated mice was 114% of that of the normal (untreated) mice (P=0.48, 8.3±3.2 versus 7.3±2.5 large vessels/mm²). The medium vessel density in PDGF-CC treated mice was 111% of that of the normal (untreated) mice (P=0.53, 14.5±3.7 versus 13±4.7 medium vessels/mm²). The small vessel density in PDGF-CC treated mice was 159.4% of that of the normal (untreated) mice (P=0.038, 33.2±12.5 versus 20.8±9.7 small vessels/mm²).

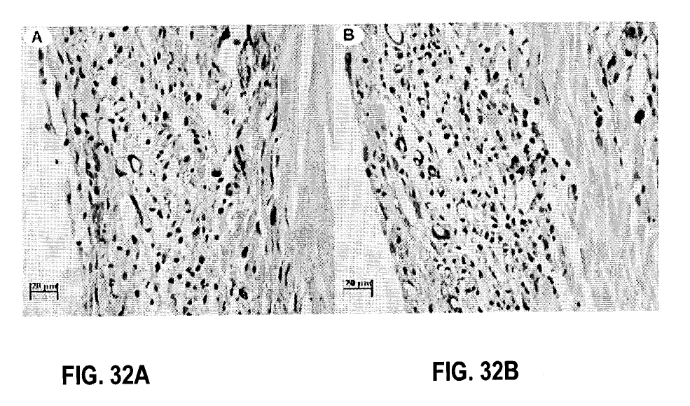

FIG. 32 shows smooth muscle actin (SMA) staining in normal (A) and PDGF-CC treated (B) hearts after infarction. The smooth muscle cell marker stains smooth muscle cells surrounding the vessels. In the infarcted area of the PDGF-CC treated mice (B), there are more positive stainings of small sized vessels compared with those in the infarcted area of untreated hearts (A).

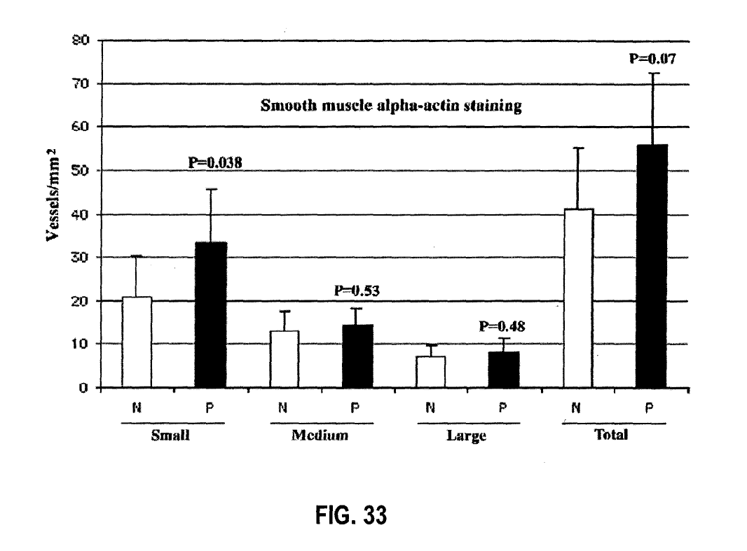

FIG. 33 shows average data for vessel densities in the infarcted area. All vessel sizes showed increased presence in the PDGF-CC treated mice. The difference in small vesels was statistically significant (P=0.038). Data are presented as average±standard deviation (SD). Open bars represent non-treated, and solid bars represent treated groups.

### EXAMPLE 17

### PDGF-C Promoted Revascularization Following Heart Infarction In Dose-Dependent Manner

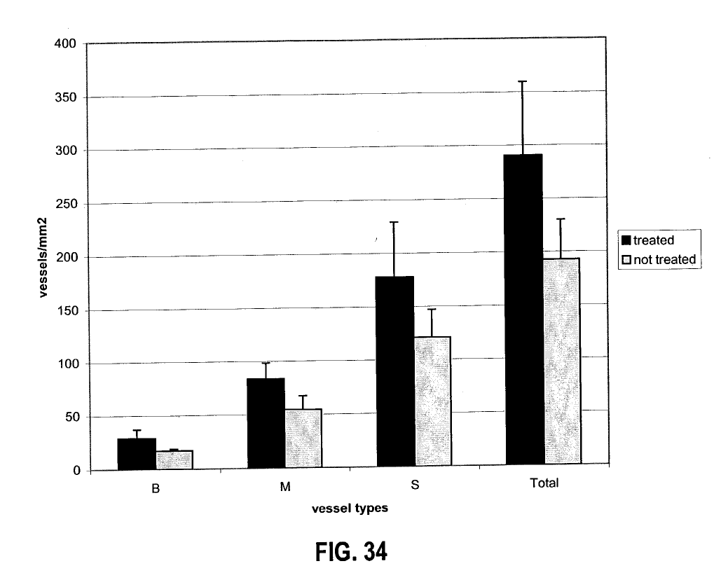

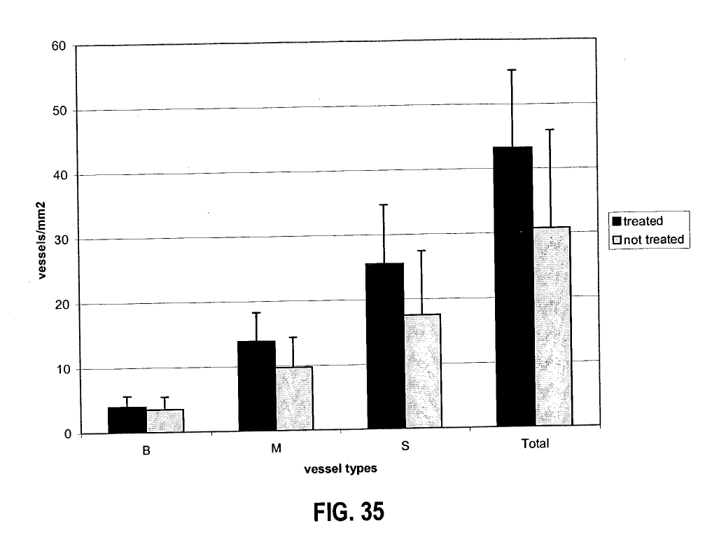

The same experiment as discussed in Example 16 was repeated using 30 μg recombinant PDGF-C per mouse. The results are shown in FIGS. 34 and 35.

FIG. 34 shows capillary density in the infarcted area 7 days following the induction of myocardial infarction in mice, treated (solid bars) or un-treated (open bars) with 30 μg of recombinant PDGF-C delivered via a mini-osmotic pump.

FIG. 35 shows the density of smooth muscle α-actin coated vessels in the infarcted area 7 days following the induction of myocardial infarction in mice, treated (solid bars) or un-treated (open bars) with 30 μg of recombinant PDGF-C delivered via a mini-osmotic pump.

Total thrombomodulin positive vessels in PDGF-C treated mice had a density 151% of that of normal (untreated) mice. The density of large, medium, small vessels in PDGF-CC treated mice are 167%, 153%, and 147%, respectively, of those of normal (untreated) mice.

Total SMA positive vessels in PDGF-CC treated mice had a density 141% of that of normal (untreated) mice. The density of large, medium, small vessels are 114%, 142%, and 145% respectively, of those of normal (untreated) mice. The results showed that treatment with 30 μg per mouse over the 7 days significantly stimulated revascularization of the infarcted area, and the stimulation was more significant than treatment with 10 μg per mouse. All vessel types seemed to respond to the treatment. Combined with the data shown in Example 16, these Example shows that PDGF-C stimulates revascularization of infarcted areas in a dose-dependent manner, and supports the conclusion that PDGF-C is useful in treating myocardinal ischemia.

Materials and Methods for Examples 18-24

1. Animal models, Recombinant Protein and Histology

Acute myocardial ischemia and hind limb ischemia mouse models were performed as previously described (Luttun et al., Nat Med, 2002 1:1; Heymans et al., Nat Med, 1999 5:1135-42). Subcutaneously implanted osmotic minipumps (Alzet, type 2001) were used for continuous protein delivery for 7 days. Human PDGF-CC core domain protein was produced as described (Li et al., Nat Cell Biol, 2000 2:302-309). Fluorescent or color dye microspheres (yellow, 15 μm, Molecular Probes) were administered after maximal vasodilatation (sodium nitroprusside, 50 ng/ml, Sigma) for blood flow measurement, and flow was calculated as described (Carmeliet et al., Nat Med, 1999 5:495-502). For histology, the hearts were harvested seven days after LAD ligation, and sectioned longitudinally (6 μm). Infarcted areas were morphologically inspected after immunohistochemistry staining using thrombomodulin (rabbit anti-TM, for all vessels) and smooth muscle alpha-actin (mouse anti-SMA, for mature SMC covered vessels, Dako), and vessel densities calculated. Gastrocnemius muscles after femoral artery ligation were sectioned transversally and analysed after H&E or immunostainings with the EC marker CD31 (PECAM, rat anti-CD31, Pharmingen). Vessel densities and tissue necrosis/regeneration in the gastrocnemius muscle were analyzed morphometrically using the KS300 image analysis soft ware (Zeiss). Remodeling of collateral vessels in the upper hindlimb after femoral ligation was quantified as reported (Luttun et al., Nat Med, 2002 1:1).

2. EPC Mobilization Assay

For EPC mobilization assay, mice were treated with PDGF-CC protein (4.5 μg/day) immediately after femoral artery ligation using subcutaneously implanted osmotic minipumps (Alzet, type 2001). After two or five days, mice were sacrificed and spleens harvested for EPC analysis using procedures described previously (Asahara et al., Circ Res, 1999 85:221-8; Dimmeler et al., J. Clin. Invest., 2001 108:391-397). Spleens were mechanically minced using syringe plungers and laid over Ficoll to isolate splenocytes. Splenocytes were seeded into fibronectin-coated 24-well plates in 0.5 ml EBM medium. After three weeks of culturing, adherent cells were stained for DiI-Ac-LDL/lectin and number of the positive cells counted. Late outgrowth EPCs (after 3 weeks of culture) were identified by metabolic uptake of DiI-acetylated-LDL (Molecular Probes) and positive staining of Alexa 488-labeled isolectin B4 (Molecular Probes). Quantification of the EPC density was performed by confocal microscopy in five microscopic fields at 200× magnification, and average EPC density calculated.

3. Human Bone Marrow Cell Adherence, Viability and Differentiation Assay

Enriched human BM derived AC133+CD34+ cells (Clonetics) at 10⁵/ml were cultured for 3 days in HPGM (Clonetics) in a 6-well plate (Becton Dickinson). Cells were then seeded in collagen coated 12-well plates in EBM (Clonetics) medium containing 4% FCS and VEGF₁₆₅ (R&D Systems) or PDG-FCC (50 ng/ml each). Growth factors were added every two days and media were refreshed at 75% every four days. For adherence assay, 2.5×10⁴ of non-adherent cells/ml were cultured in the same condition on chamber slides coated with collagen, or in 96-well plate coated with 0.3% gelatin in PBS. Cells were then washed three times with PBS, fixed and stained with May-Grüinwald Giemsa (Sigma) after two weeks of culture on chamber slides (Becton Dickinson). The number of viable cells was estimated by ATP quantification using cellTiter-glo luminescent cell viability assay (Promega) according to the manufacturer's instructions. For cell surface marker staining, cells (2×10⁴/well) cultured on collagen-coated culture slides for two or four weeks were fixed (45 min, 25° C.) and permeabilized (45 min, 25° C.) using a Intrastain Kit (DAKO), and then labeled with CD31-FITC (Becton Dickinson), CD144-FITC (Pharmingen), CD34-FITC (Becton Dickinson) or SMC-Actin CY3 (Sigma). Single or double-labeled cells were analyzed using laser confocal immunofluorescence microscopy.

4. Cell Migration, Proliferation and Aortic Ring Assay

Cell migration assays were performed on growth arrested confluent HMVEC, BAEC or hSMC cells. Cell monolayers were wounded with a rubber policeman and washed with serum-free medium. Dishes were then incubated for 20 hours in serum-free medium containing VEGF₁₆₅, PDGF-AA, (R&D Systems, Minneapolis USA) or PDGF-CC. Each assay included two dishes per condition and was repeated three times independently. Cells were photographed at 40× magnification, and migration percentage corresponding to the ratio between area of the cells and the total area of the wound (Biocom visiol@b 2000 version 4.52, San Diego). For HMVEC proliferation assay, cells were seeded in 96-well plates (5 wells per condition), and incubated with VEGF, PDGF-AA, or PDGF-CC (50 ng/ml) after serum starvation. After 7 days, viable cells were counted using the cell Titer-glo luminescent cell viability assay. For NIH-3T3 and hSMC proliferation assay, cells cultured in 96-well plates were serum-starved overnight, followed by treatment with growth factors at different concentrations. Two days later, cell numbers were counted and proliferation percentage calculated, using cells cultured in medium containing 10% serum as control. Aortic ring assay was performed as described ⁵¹. Briefly, one-millimeter long aortic rings were embedded in gels of rat tail interstitial collagen and cultured at 37° C., supplemented with different growth factors (50 ng/ml). Aortic rings were analysed at day 9 of culturing. Experiments included three explants per condition and were repeated at least twice. Aortic rings were photographed at 25× magnification.

5. Gene Expression, Western Blot and Receptor Activation

RNase protection analysis (RPA) was performed according to the manufacturer's protocol (Ambion) to investigate gene expression at mRNA level. Riboprobes were prepared using RNA polymerase (Promega) and ³²P-UTP (Amersham). Mouse β-actin cDNA (250 bp, Ambion) was used as an internal control. For Western blot assay, subconfluent cells were rinsed with cold PBS supplemented with 5 g/ml of antiprotease cocktail, lysed in RIPA buffer and analyzed on 10% acrylamide SDS PAGE in reducing condition. Two antibodies to PDGFR-α (rabbit polyclonal antibody, dilution: 1/500, Santa Cruz, sc431; and monoclonal peroxidase-labeled anti-rabbit antibody, dilution: 1/2500, Sigma, A2074) were used for PDGFR-α protein detection. Membranes were developed using the Supersignal System (Pierce). For receptor activation, tissue/cell lysates were subjected to immunoprecipitation using the rabbit anti-PDGFR-α antibody. The precipitants were analysed on SDS-PAGE, and immunoblotted using a monoclonal anti-phosphotyrosine antibody (Santa Cruz).

6. PDGF-C Over-Expression and VEGF Protein Immunoassay

For PDGF-C over-expression, mouse full-length PDGF-C cDNA was cloned into pcDNA3.1/zeo(+) mammalian expression vector (Invitrogen) and the construct was verified by sequencing. Plasmid DNA was transfected into semiconfluent cells using Lipofectamine plus reagent according to manufacturers protocol (Life technology). Stable transfectants were selected with 700 μg ml⁻¹ Zeocin (Invitrogen) for 3 weeks. Resistant colonies were pooled and maintained in medium supplemented with 300 μg ml⁻¹ Zeocin.

For PDGF-CC Western blot assay, cells were starved in serum-free medium overnight. Conditioned media (overnight) were collected and protein concentration determined. Thirty-five μg protein was trichloroacetic acid (TCA) precipitated and subjected to Western blot using affinity purified polyclonal rabbit antibodies against PDGF-CC ²⁰. All the samples were in triplicates and the experiment was repeated twice. Secreted VEGF protein was quantified using the Quantikine immunoassay kit (R&D system) according to the manufacturers protocol.

7. Statistics

Two-tailed Student T-test was used for data analysis, with P<0.05 considered statistically significant. For cell migration assay, ANOVA Dunett's test was used for data analyzing, with P<0.05 considered statistically significant.

### EXAMPLE 18

### PDGF-CC Stimulates Angiogenesis and Arteriogenesis in the Ischemic Heart

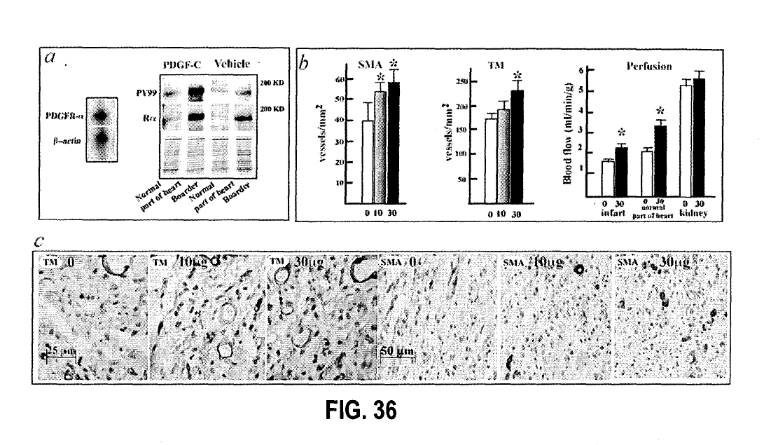

A previously established mouse model of myocardial ischemia was used to assess whether PDGF-CC is capable of stimulating the revascularization of ischemic myocardium. After coronary ligation, new vessels revascularize the ischemic core from its surrounding border region. For PDGF-CC to stimulate new vessel growth, its receptor, PDGFR-α, should be expressed in the heart. By RNAse protection analysis, PDGFR-α transcripts were detectable in the normal myocardium (FIG. 36a). Moreover, immunoprecipitation and subsequent Western blotting using an equal amount of protein extract revealed that PDGFR-α protein levels were significantly upregulated in the ischemic border zones surrounding the infarcts, i.e. where vessel growth is most active, as compared to the rest of the normal myocardium (FIG. 36b, PDGFR-α). PDGFR-α was, as assessed by Western blotting of the phosphorylated tyrosine residues after immunoprecipitation, highly activated in the border zone surrounding the infarcts (FIG. 36b, pTyr). To examine whether PDGF-CC could stimulate revascularization of the ischemic myocardium, we delivered, using a minipump, continuously over one week after coronary ligation, recombinant human PDGF-CC core domain protein, which is known to bind and activate PDGFR-α (Li et al., Nat Cell Biol, 2000 2:302-309). Compared to control, PDGF-CC indeed increased the amount of active PDGFR-α in the border region (FIG. 36b). After seven days, angiogenesis was quantified by counting the number of endothelial cell (EC)-lined vessels in the ischemic area after immunolabeling with thrombomodulin (TM). Vessel maturation (arteriogenesis) was evaluated by counting the arterioles, immunoreactive for smooth muscle cell α-actin (SMA). At 1.5 μg/day, PDGF-CC minimally affected the TM-positive vessel density (FIG. 36c,e,f) but increased, by 1.36-fold, the number of SMA-positive arterioles (FIG. 36d,h,i) (SMA positive vessels/mm²: 53.1±3.7 after PDGFC vs 38.6±4.8 after saline, n=15,16, P=0.02). When using a 3-fold higher dose (4.5 μg/day), PDGF-CC significantly stimulated angiogenesis (FIG. 36c,e,g) and arteriogenesis (FIG. 36d,h,j). No signs of hemorrhage, edema or fibrosis were observed in the PDGF-CC treated hearts. These new vessels were functional as perfusion of the ischemic myocardial region was significantly increased (blood flow: 1.6±0.2 ml/min/g in control versus 2.2±0.2 ml/min/g after 4.5 μg/day PDGF-CC; n=7-9; P<0.05). The effect of PDGF-CC to stimulate revascularization appeared to be restricted to the ischemic heart, as no differences were observed in vessel density in other organs (not shown). The magnitude of the potential of PDGF-CC to stimulate revascularization of the ischemic myocardium parallels that of VEGF and PlGF (Luttun et al., Nat Med, 2002 1:1). The mice tolerated the PDGF-CC treatment without problems, appeared healthy and had no signs of toxicity (weight loss, inactivity). Thus, PDGF-CC protein treatment promoted functional revascularization in cardiac ischemia via enhanced angiogenesis (more vessels) and arteriogenesis (more SMC coverage). The angio/arteriogenic activity of PDGF-CC in cardiac ischemia is remarkable, since the other PDGFR-α ligand, PDGF-AA is poorly angiogenic or even suppresses angiogenesis (De Marchis et al., Blood, 2002 99:2045-2053; Palumbo et al., Arterioscler Thromb Vasc Biol, 2002 22:405-11; Koyama et al., J Cell Physiol, 1994 158:1-6).

### EXAMPLE 19

### PDGF-CC Stimulates Angiogenesis in the Ischemic Limb

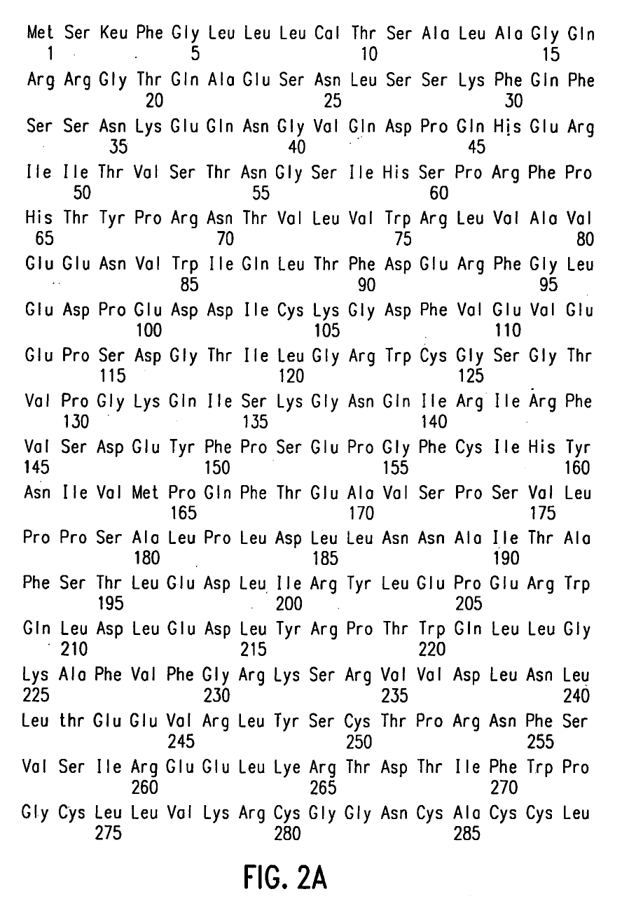

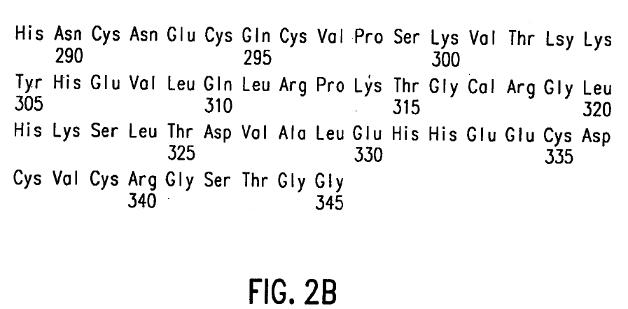

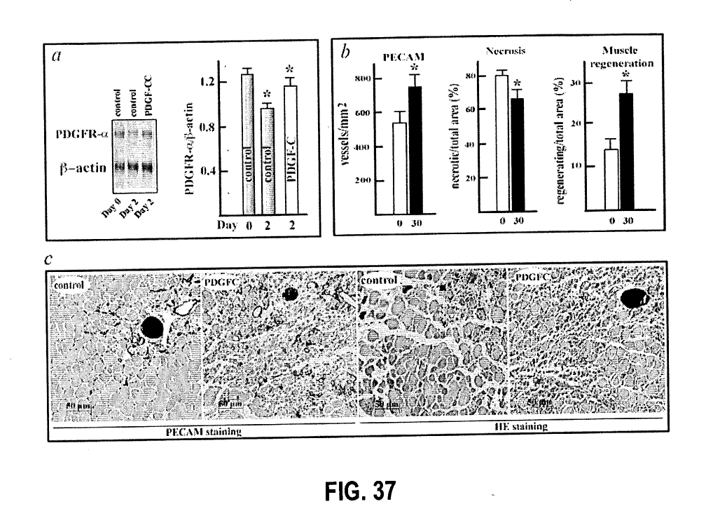

To further verify the angio/arteriogenic activity of PDGF-CC in vivo, the effect of PDGF-CC in an established mouse model of hind limb ischemia is investigated (Luttun et al., Nat Med, 2002 1:1). PDGFR-α expression was quantified by RNAse protection analysis in the gastrocnemius muscle, which becomes highly ischemic after ligation of the femoral artery (Deindl et al., Circ Res, 2001 89:779-86; Couffinhal et al., American Journal of Pathology, 1998 152:1667-1679). Two days after femoral artery ligation, when a fraction of myocytes died due to ischemic necrosis, PDGFR-α transcript levels decreased to 76% of those found in normal muscles (FIG. 37a). However, compared to vehicle, a daily treatment with 4.5 μg PDGF-CC upregulated PDGFR-α expression at day 2 after ligation and almost completely restored its expression levels to those found in the unligated control muscle (FIG. 37a). Revascularization of the ischemic gastrocnemius muscle, which only occurred in those regions where regenerating muscle replaced the necrotic avascular muscle, was scored after continuous delivery, by osmotic minipump, of 4.5 μg PDGF-CC per day for one week after femoral artery ligation. Treatment with PDGF-CC after femoral artery ligation not only increased angiogenesis (e.g. the capillary density; FIG. 37b,f,g), it also enhanced arteriogenesis (e.g. the density of SMA⁺ vessels; FIG. 37c). Moreover, PDGF-CC enhanced skeletal muscle regeneration (FIG. 2e,h-l) and, as a result, also reduced the extent of ischemic muscle necrosis (FIG. 37d,h-l), suggesting that muscle regeneration and angiogenesis might be linked. PDGF-CC also enlarged the second-generation collateral side branches in the adductor muscle (680±40 μm³ after saline versus 920±100 μm³ after PDGF-CC; N=10; P=0.05). No signs of hemorrhage, edema or fibrosis were observed in the PDGF-CC treated limbs. Thus, PDGF-CC stimulates revascularization in mouse models of both heart and limb ischemia.

### EXAMPLE 20

### PDGF-CC Mobilizes Endothelial Progenitors Upon Tissue Ischemia In Vivo

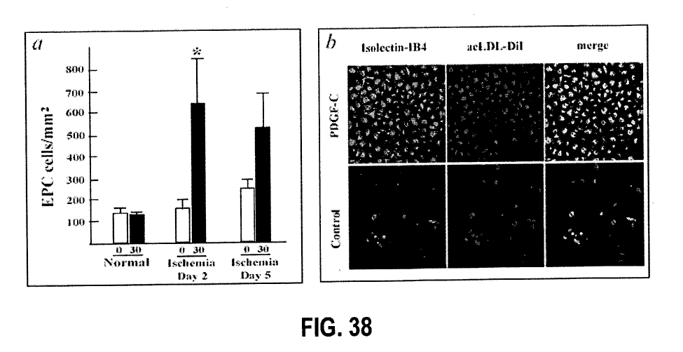

To examine the possible mechanism of how PDGF-CC stimulates vessel growth and maturation, we next assessed its effects on vascular endothelial progenitor cells (EPCs). Several vascular growth factors, such as VEGF and PlGF, have been shown to mobilize vascular stem/progenitor cells to sites of vessel growth and tissue repair ((Kalka et al., Ann Thorac Surg, 2000 70:829-34; Hattori et al., Nat Med, 2002 1:1; Rafii et al., Semin Cell Dev Biol, 2002 13:61-7; Asahara et al., Embo J, 1999 18:3964-72; Carmeliet et al., Thromb Haemost, 2001 86:289-97). A possible role of PDGF-CC in EPC mobilization has, however, not been investigated thus far. We quantified EPC mobilization by counting the number of acLDL-DiI/isolectin-IB4 positive endothelial cells after 3 weeks of plating spleen mononuclear cells. By scoring after 3 weeks, only late-outgrowth EPCs, but not surviving sloughed-off endothelial cells, are selectively assayed (Lin et al., J Clin Invest, 2000 105:71-7; Rafii, S., J Clin Invest, 2000 105:17-9). In baseline conditions, PDGF-CC did not affect mobilization of EPCs (FIG. 38a), consistent with our observation that PDGF-CC did not affect vessel growth in non-ischemic organs but only in ischemic tissues (see above). We therefore ligated the femoral arteries and found that treatment with PDGF-CC for two days (4.5 μg/day via minipump) augmented EPC mobilization approximately 3-fold above the levels found in the control group (FIGS. 38a-g). This augmentation persisted to day five, albeit at a lower level (FIG. 38a). Thus, PDGF-CC treatment enhanced EPC mobilization in tissue ischemia, thereby providing a source of ECs needed for revascularization of ischemic tissues.

### EXAMPLE 21

### PDGF-CC Enhances Differentiation of Bone Marrow Progenitors into both ECs and SMCs

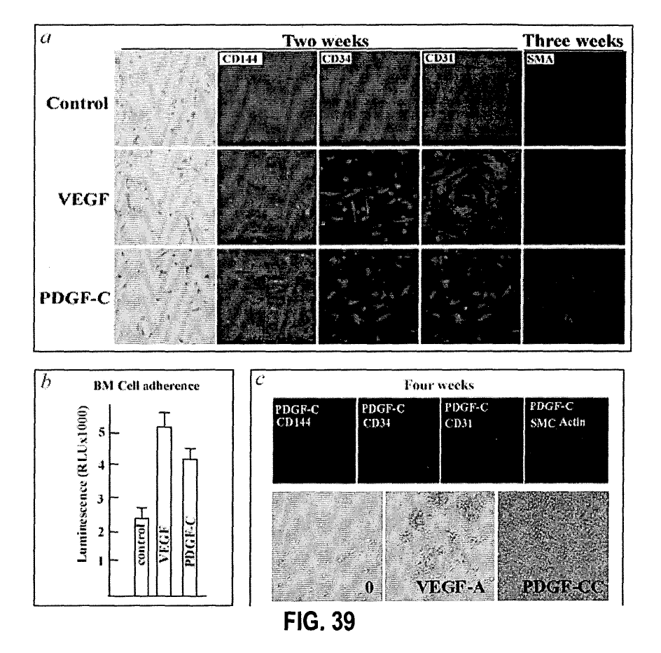

Upon stimulation by growth factors or cytokines, bone marrow stem/progenitor cells can differentiate into ECs and SMCs and thereby contribute to angio/arteriogenesis (Orlic et al., Nature, 2001 410:701-5; Kawamoto et al., Circulation, 2001 103:634-7; Asahara et al., Circ Res, 1999 85:221-8). The potential role of PDGF-CC in the differentiation of bone marrow progenitors into vascular cells has, however, not been investigated thus far. We therefore cultured human bone marrow-derived AC133⁺CD34⁺ cells—a population enriched for stem/progenitor cells (Miraglia et al., Blood, 1997 90:5013-21; Yu et al., J Biol Chem, 2002 277:20711-6; Donnelly et al., Leuk Lymphoma, 2001 40:221-34)—and stimulated them with PDGF-CC, using VEGF as a control (50 ng/ml each). These cells expressed PDGFR-α, when analyzed by RT-PCR (not shown). After two weeks of stimulation, both PDGF-CC and VEGF enhanced the adherence of these cells—a prerequisite for anchorage-dependent cell proliferation, differentiation, migration and prevention of apoptosis (Assoian, J Cell Biol, 1997 136:1-4; Asahara et al., Science, 1997 275:964-7) (FIGS. 39a-d). However, the two growth factors markedly differed in their ability to induce the commitment of these stem/progenitor cells into either the endothelial or smooth muscle cell lineage. After two weeks of stimulation, both PDGF-CC and VEGF induced the expression of the EC surface markers CD144 (VE-cadherin; FIG. 39e-g) and CD31 (PECAM; FIGS. 39h-j), indicating that these growth factors induced a characteristic endothelial phenotype. Interestingly, only PDGF-CC additionally induced the expression of the smooth muscle cell marker SMA in a fraction of these cells, indicating that these cells had acquired a characteristic SMC phenotype (FIGS. 39k-m). Notably, this pleiotropic effect of PDGF-CC in inducing both endothelial and smooth muscle lineages was specific, as the VEGF-treated cells did not become SMA positive (FIGS. 39k-m). Double labeling experiments revealed that PDGF-CC often induced the expression of CD31 and SMA in the same cells. By four weeks, most (>95%) of the PDGF-CC-treated cells were SMA positive and had lost their expression of CD144 and CD31, while VEGF-treated cells, instead, were still CD144 and CD31 positive but remained SMA negative (not shown). Thus, PDGF-CC initially induced bone marrow progenitor cells to differentiate into cell types with both endothelial or smooth muscle cell characteristics—eventually, after long-term treatment, yielding cells with a SMC-like phenotype. PDGF-CC thus differed from VEGF, as the latter only caused bone marrow progenitors to acquire EC-specific markers, even after prolonged treatment.

### EXAMPLE 22

### PDGF-CC Promotes Endothelial Cell Migration and Microvessel Sprouting

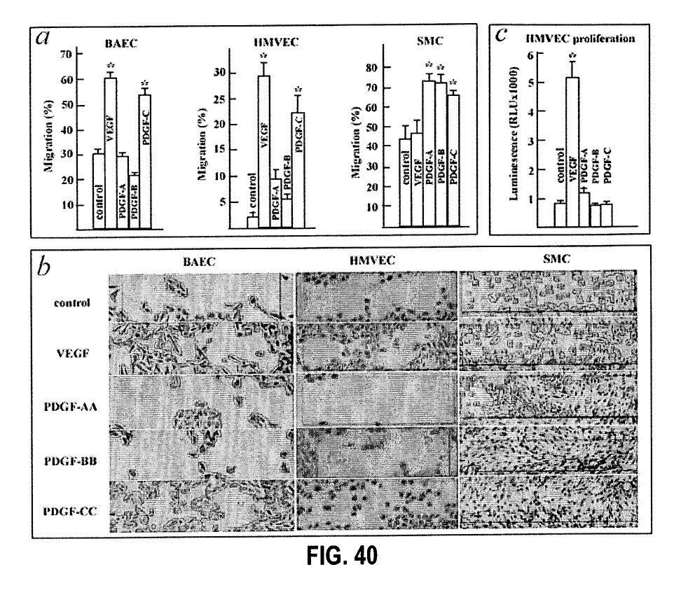

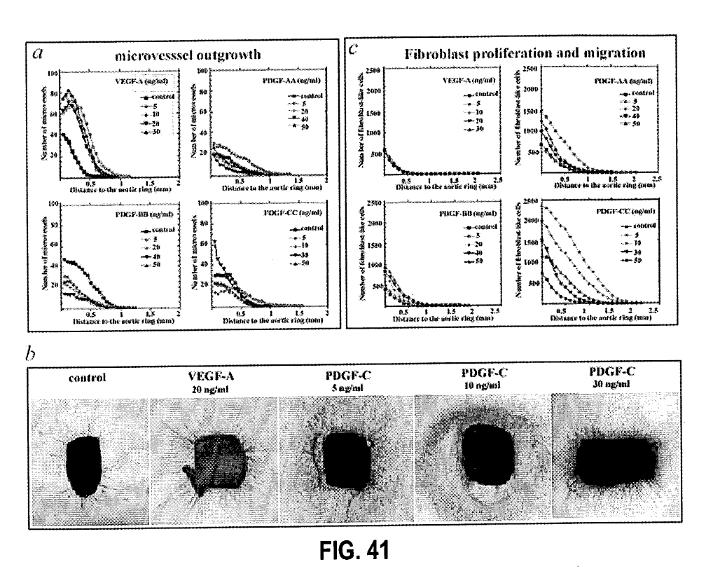

Although PDGFR-α is expressed on endothelial cells (Edelberg et al., Journal of Clinical Investigation, 1998 102:837-43; Smits et al., Growth Factors, 1989 2:1-8; Bar et al., Endocrinology, 1989 124:1841-8; Beitz et al., Proc Natl Acad Sci USA, 1991 88:2021-5; Marx et al., J Clin Invest, 1994 93:131-9; Shinbrot et al., Dev. Dyn., 1994 199:169-175), little is known about the functional consequence of PDGFR-α signaling in these cells. We therefore compared the effect of PDGF-CC on EC migration and proliferation to that of VEGF (which primarily affects endothelial cells (Senger et al., Am J Pathol, 1996 149:293-305) and PDGF-AA (which primarily affects fibroblasts and smooth muscle cells (Heldin et al., Physiol Rev, 1999 79:1283-1316)). PDGFR-A expression on the human microvascular endothelial cells (HMVEC) was confirmed by Western blot, albeit at a lower level as compared with that of the SMCs (not shown). VEGF and PDGF-CC, but not PDGF-AA, stimulated migration of HMVECs (FIG. 40a). This effect of PDGF-CC was not restricted to HMVECs only, as PDGF-CC also enhanced the migration of bovine aorta endothelial cells (BAEC; FIG. 40b). However, none of the PDGFs affected EC proliferation (FIG. 40c), in agreement with the previous observation that PDGFR-α does not transmit mitogenic signals in response to PDGF-AA in ECs (Marx et al., J Clin Invest, 1994 93:131-9). VEGF, instead, highly stimulated EC proliferation (FIG. 5c). We also tested the effect of PDGF-CC on cultured aortic rings, as this assay allows assessment of the outgrowth of microvessels from an intact vessel in vitro (Blacher et al., Angiogenesis, 2001 4:133-42). Results are graphically represented as the number of microvessels and the distance over which they grew out from the aortic ring. Each experiment included three explants per condition and was repeated at least twice. At day 9 after culturing, microvessels and the distance of their outgrowth were quantified and evaluated using Student's t test. In baseline conditions, only a small number of microvessels sprouted from the aortic rings—most of them over very short distances (0.25 mm from the aortic ring)—and only a small fraction (<5%) growing out over longer distances (>0.5 mm from the aortic ring, FIG. 41a). VEGF increased not only the number of sprouting microvessels (P<0.001 at all concentrations versus control), but also the distance over which they grew out (P<0.05 at all concentrations versus control; FIG. 41b,f). At 30 ng/ml, PDGF-CC increased the number of microvessels (P<0.001 versus control, FIG. 41e,g) and increased the distance of vessel outgrowth at 5 ng/ml (P<0.01 versus control, FIG. 41g). Apparently, PDGF-CC had its maximum effect at 30 ng/ml on microvessel sprouting, and was less potent at a concentration of 50 ng/ml, indicating that the dose-response relationship of PDGF-CC in the aortic ring assay was bell-shaped. A similar bell-shaped dose-response relationship has been documented for other members of the VEGF/PDGF-superfamily (Jin et al., J Mol Neurosci, 2000 14:197-203). PDGF-AA, however, had no effect on the number of microvessels, although it increased the distance of vessel outgrowth at 5 ng/ml (P<0.01 versus control, FIG. 41h). Thus, PDGF-CC mobilized EC migration in cultured cells and promoted microvessel sprouting in the aortic ring assay. This chemotactic effect of PDGF-CC on ECs is surprising, since although the other PDGFs are among the most potent stimuli of mesenchymal cell migration, they do not or minimally stimulate—and, in certain conditions, even inhibit—EC migration (De Marchis et al., Blood, 2002 99:2045-2053).

### EXAMPLE 23

### PDGF-CC is Both Chemotactic and Mitogenic for SMCs and Perivascular Fibroblast Cells

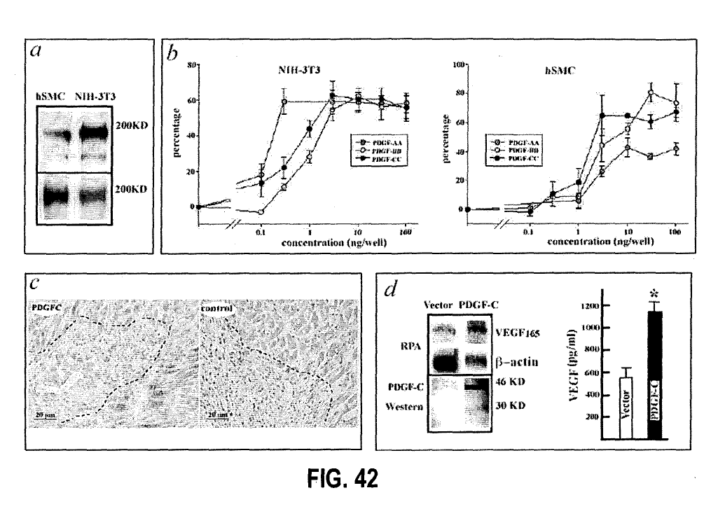

The mitogenic and chemotactic effect of PDGF-CC was then tested on SMCs and perivascular fibroblast cells, and the effect of PDGF-CC was compared to that of VEGF and PDGF-AA in both cultured cells and the aortic ring assay (Blacher et al., Angiogenesis, 2001 4:133-42). PDGF-CC-treated cultured hSMC and NIH-3T3 fibroblast cells expressed significant amounts of PDGFR-α (FIG. 42a, PDGFR-α). Subsequent immunoblotting for pTyr indicated that PDGFR-α was highly activated (FIG. 42a, pTyr). Both PDGF-CC and -AA stimulated hSMC migration with a comparable potency, while VEGF had no effect (FIG. 42b). PDGF-CC and -AA also stimulated the proliferation of cultured NIH-3T3 fibroblast and hSMC cells, the effect of PDGF-CC on the latter cells being slightly more pronounced (FIG. 42c,d). In the aortic ring assay, we quantified the growth and emigration of perivascular fibroblasts from the intact vessel using computer-assisted image analysis after treatment with VEGF and different PDGFs at different concentrations. In baseline conditions, individual perivascular fibroblast-like cells (identified as isolated cells, not associated with sprouting microvessels) were sparse and emigrated over only short distances from the aortic ring (FIG. 42a). While VEGF was ineffective on the perivascular fibroblast-like cells (FIG. 42b,i), PDGF-CC significantly increased the number of such cells, which also emigrated over much greater distances from the aortic ring (P<0.001 at all concentrations versus control, FIGS. 42c-e,j). At high concentrations (30-50 ng/ml), PDGF-CC still stimulated fibroblast-like cell growth and emigration but less significantly than at lower concentrations, possibly because its effects were dose-dependent (see above) and/or the perivascular cells surrounded the sprouting microvessels. PDGF-AA had an intermediate effect on the perivascular fibroblast-like cells (P<0.05 at different concentrations versus control, FIG. 41k). Thus, PDGF-CC, more potently than PDGF-AA, stimulated the migration and proliferation of perivascular cells in the aortic ring assay—an assay that is believed to reflect more closely the in vivo situation and allows synergistic interactions between the different vascular cell types (Hartlapp et al, 2001, FASEB J, 15: 2215-24).

### EXAMPLE 25

### Bioassays to Determine the Function of PDGF-C

Assays are conducted to evaluate whether PDGF-C has similar activities to PDGF-A, PDGF-B, VEGF, VEGF-B, VEGF-C and/or VEGF-D in relation to growth and/or motility of connective tissue cells, fibroblasts, perivascular, myofibroblasts and glial cells; to endothelial cell function; to angiogenesis; and to wound healing. Further assays may also be performed, depending on the results of receptor binding distribution studies.

I. Mitogenicity of PDGF-C for Endothelial Cells

To test the mitogenic capacity of PDGF-C for endothelial cells, the PDGF-C polypeptide is introduced into cell culture medium containing 5% serum and applied to bovine aortic endothelial cells (BAEs) propagated in medium containing 10% serum. The BAEs are previously seeded in 24-well dishes at a density of 10,000 cells per well the day before addition of the PDGF-C. Three days after addition of this polypeptide the cells were dissociated with trypsin and counted. Purified VEGF is included in the experiment as positive control.

II. Assays of Endothelial Cell Function

a) Endothelial Cell Proliferation

Endothelial cell growth assays are performed by methods well known in the art, e.g. those of Ferrara & Henzel, Nature, 1989 380:439-443, Gospodarowicz et al., Proc. Natl. Acad. Sci. USA, 1989 86:7311-7315, and/or Claffey et al., Biochem. Biophys. Acta, 1995 1246:1-9.

b) Cell Adhesion Assay

The effect of PDGF-C on adhesion of polymorphonuclear granulocytes to endothelial cells is tested.

c) Chemotaxis

The standard Boyden chamber chemotaxis assay is used to test the effect of PDGF-C on chemotaxis.

d) Plasminogen Activator Assay

Endothelial cells are tested for the effect of PDGF-C on plasminogen activator and plasminogen activator inhibitor production, using the method of Pepper et al., Biochem. Biophys. Res. Commun., 1991 181:902-906.

e) Endothelial Cell Migration assay

The ability of PDGF-C to stimulate endothelial cells to migrate and form tubes is assayed as described in Montesano et al., Proc. Natl. Acad. Sci. USA, 1986 83:7297-7301. Alternatively, the three-dimensional collagen gel assay described in Joukov et al., EMBO J., 1996 15:290-298 or a gelatinized membrane in a modified Boyden chamber (Glaser et al., Nature, 1980 288:483-484) may be used.

III. Angiogenesis Assay

The ability of PDGF-C to induce an angiogenic response in chick chorioallantoic membrane is tested as described in Leung et al., Science, 1989 246 1306-1309. Alternatively the rat cornea assay of Rastinejad et al., Cell, 1989 56 345-355 may be used; this is an accepted method for assay of in vivo angiogenesis, and the results are readily transferrable to other in vivo systems.

UV. Wound Healing

The ability of PDGF-C to stimulate wound healing is tested in the most clinically relevant model available, as described in Schilling et al., Surgery, 1959 46:702-710 and utilized by Hunt et al., Surgery, 1967 114:302-307.

V. The Hemopoietic System

A variety of in vitro and in vivo assays using specific cell populations of the haemopoietic system are known in the art, and are outlined below. In particular a variety of in vitro murine stem cell assays using fluorescence-activated cell sorter to purified cells are particularly convenient:

a) Repopulating Stem Cells

These are cells capable of repopulating the bone marrow of lethally irradiated mice, and have the Lin⁻, Rh^{h1}, Ly-6A/E⁺, c-kit⁺ phenotype. PDGF-C is tested on these cells either alone, or by co-incubation with other factors, followed by measurement of cellular proliferation by ³H-thymidine incorporation.

b) Late Stage Stem Cells

These are cells that have comparatively little bone marrow repopulating ability, but can generate D13 CFU-S. These cells have the Lin⁻, Rh^{h1}, Ly-6A/E⁺, c-kit⁺ phenotype. PDGF-C is incubated with these cells for a period of time, injected into lethally irradiated recipients, and the number of D13 spleen colonies enumerated.

c) Progenitor-Enriched Cells

These are cells that respond in vitro to single growth factors and have the Lin⁻, Rh^{h1}, Ly-6A/E⁺, c-kit⁺ phenotype. This assay will show if PDGF-C can act directly on haemopoietic progenitor cells. PDGF-C is incubated with these cells in agar cultures, and the number of colonies present after 7-14 days is counted.

VI. Atherosclerosis

Smooth muscle cells play a crucial role in the development or initiation of atherosclerosis, requiring a change of their phenotype from a contractile to a synthetic state. Macrophages, endothelial cells, T lymphocytes and platelets all play a role in the development of atherosclerotic plaques by influencing the growth and phenotypic modulations of smooth muscle cell. An in vitro assay using a modified Rose chamber in which different cell types are seeded on to opposite cover slips measures the proliferative rate and phenotypic modulations of smooth muscle cells in a multicellular environment, and is used to assess the effect of PDGF-C on smooth muscle cells.

VII. Metastasis

The ability of PDGF-C to inhibit metastasis is assayed using the Lewis lung carcinoma model, for example using the method of Cao et al., J. Exp. Med., 1995 182:2069-2077.

VIII. Migration of Smooth Muscle Cells

The effects of the PDGF-C on the migration of smooth muscle cells and other cells types can be assayed using the method of Koyama et al., J. Biol. Chem., 1992 267:22806-22812.

IX. Chemotaxis

The effects of the PDGF-C on chemotaxis of fibroblast, monocytes, granulocytes and other cells can be assayed using the method of Siegbahn et al., J. Clin. Invest., 1990 85:916-920.

X. PDGF-C in Other Cell Types

The effects of PDGF-C on proliferation, differentiation and function of other cell types, such as liver cells, cardiac muscle and other cells, endocrine cells and osteoblasts can readily be assayed by methods known in the art, such as ³H-thymidine uptake by in vitro cultures. Expression of PDGF-C in these and other tissues can be measured by techniques such as Northern blotting and hybridization or by in situ hybridization.

XI. Construction of PDGF-C Variants and Analogs

PDGF-C is a member of the PDGF family of growth factors which exhibits a high degree of homology to the other members of the PDGF family. PDGF-C contains eight conserved cysteine residues which are characteristic of this family of growth factors. These conserved cysteine residues form intra-chain disulfide bonds which produce the cysteine knot structure, and inter-chain disulfide bonds that form the protein dimers which are characteristic of members of the PDGF family of growth factors. PDGF-C interacts with a protein tyrosine kinase growth factor receptor.

In contrast to proteins where little or nothing is known about the protein structure and active sites needed for receptor binding and consequent activity, the design of active mutants of PDGF-C is greatly facilitated by the fact that a great deal is known about the active sites and important amino acids of the members of the PDGF family of growth factors.

Published articles elucidating the structure/activity relationships of members of the PDGF family of growth factors include for PDGF: Oestman et al., J. Biol. Chem., 1991 266:10073-10077; Andersson et al., J. Biol. Chem., 1992 267:11260-1266; Oefner et al., EMBO J., 1992 11:3921-3926; Flemming et al., Molecular and Cell Biol., 1993 13:4066-4076 and Andersson et al., Growth Factors, 1995 12:159-164; and for VEGF: Kim et al., Growth Factors, 1992 7:5364; Pötgens et al., J. Biol. Chem., 1994 269:32879-32885 and Claffey et al., Biochem. Biophys. Acta, 1995 1246:1-9. From these publications it is apparent that because of the eight conserved cysteine residues, the members of the PDGF family of growth factors exhibit a characteristic knotted folding structure and dimerization, which result in formation of three exposed loop regions at each end of the dimerized molecule, at which the active receptor binding sites can be expected to be located.

Based on this information, a person skilled in the biotechnology arts can design PDGF-C mutants with a very high probability of retaining PDGF-C activity by conserving the eight cysteine residues responsible for the knotted folding arrangement and for dimerization, and also by conserving, or making only conservative amino acid substitutions in the likely receptor sequences in the loop 1, loop 2 and loop 3 region of the protein structure.

The formation of desired mutations at specifically targeted sites in a protein structure is considered to be a standard technique in the arsenal of the protein chemist (Kunkel et al., Methods in Enzymol., 1987 154:367-382). Examples of such site-directed mutagenesis with VEGF can be found in Pötgens et al., J. Biol. Chem., 1994 269:32879-32885 and Claffey et al., Biochem. Biophys. Acta, 1995 1246:1-9. Indeed, site-directed mutagenesis is so common that kits are commercially available to facilitate such procedures (e.g. Promega 1994-1995 Catalog., Pages 142-145).

The connective tissue cell, fibroblast, myofibroblast and glial cell growth and/or motility activity, the endothelial cell proliferation activity, the angiogenesis activity and/or the wound healing activity of PDGF-C mutants can be readily confirmed by well established screening procedures. For example, a procedure analogous to the endothelial cell mitotic assay described by Claffey et al., Biochem. Biophys. Acta., 1995 1246:1-9 can be used. Similarly the effects of PDGF-C on proliferation of other cell types, on cellular differentiation and on human metastasis can be tested using methods which are well known in the art.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

What is claimed is:

1. A pharmaceutical composition for modulating at least one of vasculogenesis, arteriogenesis and angiogenesis in a mammal, comprising an effective amount of a polypeptide having at least 85% sequence identity with the sequence of SEQ ID NO:40.

2. A pharmaceutical composition of claim 1, wherein the sequence identity is at least 90%.

3. A pharmaceutical composition of claim 1, wherein the sequence identity is at least 95%.

4. A pharmaceutical composition of claim 1, wherein the polypeptide comprising the sequence of SEQ ID NO: 40.

5. A pharmaceutical composition of claim 1, further comprising one or more of PDGF-A, PDGF-B, PDGF-D, VEGF, VEGF-B, VEGF-C, VEGF-D, PlGF and/or heparin.

6. A pharmaceutical composition of claim 1, further comprising a pharmaceutical carrier or diluent.

7. A pharmaceutical composition of claim 1, comprising from about 0.1% to 90% by weight of the polypeptide.

8. A pharmaceutical composition for modulating at least one of vasculogenesis, arteriogenesis and angiogenesis, comprising a pharmaceutically effective amount of an expression vector which comprises a polynucleotide encoding a polypeptide having at least 85% sequence identity with the sequence of SEQ ID NO:40.

9. A pharmaceutical composition for modulating at least one of vasculogenesis, arteriogenesis and angiogenesis, comprising a pharmaceutically effective amount of a polypeptide dimer comprising a polypeptide having at least 85% sequence identity with the sequence of SEQ ID NO:40.

10. A pharmaceutical composition of claim 9, wherein the dimer is a heterodimer comprising an active monomer of VEGF, VEGF-B, VEGF-C, VEGF-D, PDGF-C, PDGF-A, PDGF-B, PDGF-D or PlGF and an active monomer of PDGF-C.

11. A pharmaceutical composition of claim 9, wherein the dimer is PDGF-CC.

12. A method for modulating at least one of vasculogenesis, angiogenesis and arteriogenesis, said method comprising administering to a subject in need thereof a pharmaceutically effective amount of a polypeptide having at least 85% sequence identity with the sequence of SEQ ID NO:40.

13. A method according to claim 12, wherein the sequence identity is at least 90%.

14. A method according to claim 12, wherein the sequence identity is at least 95%.

15. A method according to claim 12, wherein the polypeptide comprises the sequence of SEQ ID NO: 40.

16. A method of claim 12, wherein the method is for treating ischemia.

17. A method according to claim 16, wherein the method is for treating myocardial ischemia.

18. A method according to claim 16, wherein the method is for treating limb ischemia.

19. A method of claim 12, wherein at least one of vasculogenesis, angiogenesis and arteriogenesis in the subject are increased.

20. A method of claim 16, wherein at least one of angiogenesis, arteriogenesis and arteriogenesis are increased in ischemic tissue of the subject.

21. A method of claim 16, wherein the polypeptide is administered to ischemic tissue of the subject.

22. A method of claim 21, wherein the polypeptide is injected into the ischemic tissue of the subject.

23. A method of claim 22, wherein the polypeptide is injected into the ischemic tissue of the subject via a subcutaneous minipump.

24. A method according to claim 12, wherein the polypeptide is a homodimer of activated PDGF-C.

25. A method for modulating at least one of vasculogenesis, arteriogenesis and angiogenesis, said method comprising administering to a subject in need thereof a pharmaceutically effective amount of a polynucleotide encoding a polypeptide having at least 85% sequence identity with the sequence of SEQ ID NO:40.

26. A method of claim 25, wherein the polynucleotide is a suitable expression vector.

27. A method of claim 25, wherein the method is for treating ischemia

28. A method according to claim 27, wherein the method is for treating myocardial ischemia.

29. A method according to claim 27, wherein the method is for treating limb ischemia.

30. A method of claim 25, wherein at least one of vasculogenesis, arteriogenesis and angiogenesis in the subject is increased.

31. A method of claim 27, wherein at least one of vasculogenesis, angiogenesis and arteriogenesis is increased in ischemic tissue of the subject.

32. A method of claim 27, wherein the polynucleotide is administered to ischemic tissue of the subject.

33. A method of claim 32, wherein the polynucleotide is injected into the ischemic tissue of the subject.

34. A method of claim 33, wherein the polypeptide is injected into the ischemic tissue of the subject via a subcutaneous minipump.

35. A method for improving abnormal cardiac function in a mammal, which comprises:
a) injecting into heart muscle of said mammal a DNA encoding a polypeptide having at least 85% sequence identity with the sequence of SEQ ID NO:40.
b) obtaining expression of said polypeptide in said heart muscle in an amount that increases vasculogenesis or arteriogenesis or angiogenesis within the heart muscle, thereby improving cardiac function.

36. A method for modulating at least one of vasculogenesis, angiogenesis and arteriogenesis, said method comprising administering to a subject in need thereof, the method comprising administering to a subject in need thereof an effective amount of a polypeptide having at least 85% sequence identity with the sequence of SEQ ID NO:40, or a polynucleotide encoding said polypeptide.

37. A method according to claim 36, wherein differentiation of bone marrow cells into smooth muscle cells or endothelial cells or both in said subject is promoted.

38. A method according to claim 36, wherein muscle regeneration in ischemic tissue of said subject is stimulated.

39. A method according to claim 36, wherein vessel density in said subject is increased.

40. A method according to claim 36, wherein mobilization of endothelial progenitor cells in said subject is promoted.

41. A method according to claim 36, wherein said subject has an ischemic brain, and wherein neoangiogenesis in said ischemic brain is promoted.

42. The method of claim 41, wherein the subject in need thereof is a post-stroke patient.
